SEQUENCE LISTING (299 sequences)

SEQ ID NO: 1 - protein, 12 aa, Artificial Sequence
  Synthetic sequence TCR1007 CDR3a
CAVPNTGNQFYF

SEQ ID NO: 2 - protein, 7 aa, Artificial Sequence
  Synthetic sequence TCR1007 CDR2a
IRPDVSE

SEQ ID NO: 3 - protein, 6 aa, Artificial Sequence
  Synthetic sequence TCR1007 CDR1a
NTAFDY

SEQ ID NO: 4 - protein, 15 aa, Artificial Sequence
  Synthetic sequence TCR1007 CDR3b
CASSLIAGLSYEQYF

SEQ ID NO: 5 - protein, 6 aa, Artificial Sequence
  Synthetic sequence TCR1007 CDR2b
FQNEAQ

SEQ ID NO: 6 - protein, 5 aa, Artificial Sequence
  Synthetic sequence TCR1007 CDR1b
SGHVS

SEQ ID NO: 7 - protein, 15 aa, Artificial Sequence
  Synthetic sequence TCR1009 CDR3a
CALSLPYTNAGKSTF

SEQ ID NO: 8 - protein, 8 aa, Artificial Sequence
  Synthetic sequence TCR1009 CDR2a
RNSFDEQN

SEQ ID NO: 9 - protein, 7 aa, Artificial Sequence
  Synthetic sequence TCR1009 CDR1a
TRDTTYY

SEQ ID NO: 10 - protein, 15 aa, Artificial Sequence
  Synthetic sequence TCR1009 CDR3b
CASSDRQGSNQPQHF

SEQ ID NO: 11 - protein, 6 aa, Artificial Sequence
  Synthetic sequence TCR1009 CDR2b
SASEGT

SEQ ID NO: 12 - protein, 5 aa, Artificial Sequence
  Synthetic sequence TCR1009 CDR1b
MNHNS

SEQ ID NO: 13 - protein, 15 aa, Artificial Sequence
  Synthetic sequence TCR1012 CDR3a
CAVRDDGGGGNKLTF

SEQ ID NO: 14 - protein, 8 aa, Artificial Sequence
  Synthetic sequence TCR1012 CDR2a
YITGDNLV

SEQ ID NO: 15 - protein, 6 aa, Artificial Sequence
  Synthetic sequence TCR1012 CDR1a
VSGNPY

SEQ ID NO: 16 - protein, 15 aa, Artificial Sequence
  Synthetic sequence TCR1012 CDR3b
CASSSSGGPGYEQYF

SEQ ID NO: 17 - protein, 6 aa, Artificial Sequence
  Synthetic sequence TCR1012 CDR2b
FQNEAQ

SEQ ID NO: 18 - protein, 5 aa, Artificial Sequence
  Synthetic sequence TCR1012 CDR1b
SGHVS

SEQ ID NO: 19 - protein, 15 aa, Artificial Sequence
  Synthetic sequence TCR1016 CDR3a
CAMRERGGGYQKVTF

SEQ ID NO: 20 - protein, 8 aa, Artificial Sequence
  Synthetic sequence TCR1016 CDR2a
QGSYDQQN

SEQ ID NO: 21 - protein, 7 aa, Artificial Sequence
  Synthetic sequence TCR1016 CDR1a
TSDPSYG

SEQ ID NO: 22 - protein, 16 aa, Artificial Sequence
  Synthetic sequence TCR1016 CDR3b
CASSLLRTGEYNEQFF

SEQ ID NO: 23 - protein, 6 aa, Artificial Sequence
  Synthetic sequence TCR1016 CDR2b
FYEKMQ

SEQ ID NO: 24 - protein, 5 aa, Artificial Sequence
  Synthetic sequence TCR1016 CDR1b
PRHDT

SEQ ID NO: 25 - protein, 15 aa, Artificial Sequence
  Synthetic sequence TCR1021 CDR3a
CVVSDGDSSASKIIF

SEQ ID NO: 26 - protein, 8 aa, Artificial Sequence
  Synthetic sequence TCR1021 CDR2a
YTSAATLV

SEQ ID NO: 27 - protein, 6 aa, Artificial Sequence
  Synthetic sequence TCR1021 CDR1a
SSYSPS

SEQ ID NO: 28 - protein, 17 aa, Artificial Sequence
  Synthetic sequence TCR1021 CDR3b
CASSYLLAGGPDNEQFF

SEQ ID NO: 29 - protein, 6 aa, Artificial Sequence
  Synthetic sequence TCR1021 CDR2b
SVGAGI

SEQ ID NO: 30 - protein, 5 aa, Artificial Sequence
  Synthetic sequence TCR1021 CDR1b
MNHNY

SEQ ID NO: 31 - protein, 13 aa, Artificial Sequence
  Synthetic sequence TCR1027 CDR3a
CAVDENTGNQFYF

SEQ ID NO: 32 - protein, 6 aa, Artificial Sequence
  Synthetic sequence TCR1027 CDR2a
IYSNGD

SEQ ID NO: 33 - protein, 6 aa, Artificial Sequence
  Synthetic sequence TCR1027 CDR1a
DRGSQS

SEQ ID NO: 34 - protein, 13 aa, Artificial Sequence
  Synthetic sequence TCR1027 CDR3b
CASRIGTSQEQYF

SEQ ID NO: 35 - protein, 6 aa, Artificial Sequence
  Synthetic sequence TCR1027 CDR2b
YYEKEE

SEQ ID NO: 36 - protein, 5 aa, Artificial Sequence
  Synthetic sequence TCR1027 CDR1b
SGHKS

SEQ ID NO: 37 - protein, 11 aa, Artificial Sequence
  Synthetic sequence TCR1034 CDR3a
CLPSNDYKLSF

SEQ ID NO: 38 - protein, 7 aa, Artificial Sequence
  Synthetic sequence TCR1034 CDR2a
LVKSGEV

SEQ ID NO: 39 - protein, 5 aa, Artificial Sequence
  Synthetic sequence TCR1034 CDR1a
TTLSN

SEQ ID NO: 40 - protein, 15 aa, Artificial Sequence
  Synthetic sequence TCR1034 CDR3b
CASYRDTSSYNEQFF

SEQ ID NO: 41 - protein, 6 aa, Artificial Sequence
  Synthetic sequence TCR1034 CDR2b
FNNNVP

SEQ ID NO: 42 - protein, 5 aa, Artificial Sequence
  Synthetic sequence TCR1034 CDR1b
SGHNS

SEQ ID NO: 43 - protein, 12 aa, Artificial Sequence
  Synthetic sequence TCR1042 CDR3a
CALRYGNNRLAF

SEQ ID NO: 44 - protein, 7 aa, Artificial Sequence
  Synthetic sequence TCR1042 CDR2a
ATKADDK

SEQ ID NO: 45 - protein, 6 aa, Artificial Sequence
  Synthetic sequence TCR1042 CDR1a
ATGYPS

SEQ ID NO: 46 - protein, 16 aa, Artificial Sequence
  Synthetic sequence TCR1042 CDR3b
CASSQEWRRLADEQFF

SEQ ID NO: 47 - protein, 6 aa, Artificial Sequence
  Synthetic sequence TCR1042 CDR2b
YNNKEL

SEQ ID NO: 48 - protein, 5 aa, Artificial Sequence
  Synthetic sequence TCR1042 CDR1b
LGHDT

SEQ ID NO: 49 - protein, 12 aa, Artificial Sequence
  Synthetic sequence TCR1051 CDR3a
CAVSSGTYKYIF

SEQ ID NO: 50 - protein, 8 aa, Artificial Sequence
  Synthetic sequence TCR1051 CDR2a
YLSGSTLV

SEQ ID NO: 51 - protein, 6 aa, Artificial Sequence
  Synthetic sequence TCR1051 CDR1a
SSVSVY

SEQ ID NO: 52 - protein, 15 aa, Artificial Sequence
  Synthetic sequence TCR1051 CDR3b
CASSPGTGGNTEAFF

SEQ ID NO: 53 - protein, 6 aa, Artificial Sequence
  Synthetic sequence TCR1051 CDR2b
YFSETQ

SEQ ID NO: 54 - protein, 5 aa, Artificial Sequence
  Synthetic sequence TCR1051 CDR1b
SGHRS

SEQ ID NO: 55 - protein, 13 aa, Artificial Sequence
  Synthetic sequence TCR1061 CDR3a
CAIMTGTASKLTF

SEQ ID NO: 56 - protein, 8 aa, Artificial Sequence
  Synthetic sequence TCR1061 CDR2a
QEAYKQQN

SEQ ID NO: 57 - protein, 7 aa, Artificial Sequence
  Synthetic sequence TCR1061 CDR1a
TSESDYY

SEQ ID NO: 58 - protein, 14 aa, Artificial Sequence
  Synthetic sequence TCR1061 CDR3b
CASSSPSGANVLTF

SEQ ID NO: 59 - protein, 6 aa, Artificial Sequence
  Synthetic sequence TCR1061 CDR2b
FQNEAQ

SEQ ID NO: 60 - protein, 5 aa, Artificial Sequence
  Synthetic sequence TCR1061 CDR1b
SGHVS

SEQ ID NO: 61 - protein, 13 aa, Artificial Sequence
  Synthetic sequence TCR1072 CDR3a
CVVTGTGGFKTIF

SEQ ID NO: 62 - protein, 18 aa, Artificial Sequence
  Synthetic sequence TCR1072 CDR3b
CASSSMSIAAGNTGELFF

SEQ ID NO: 63 - protein, 139 aa, Artificial Sequence
  Synthetic sequence TCR1007 Va
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAVPNTGNQFYFGTGTSLTVIP

SEQ ID NO: 64 - protein, 134 aa, Artificial Sequence
  Synthetic sequence TCR1007 Vb
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLIAGLSYEQYFGPGTRLTVT

SEQ ID NO: 65 - protein, 136 aa, Artificial Sequence
  Synthetic sequence TCR1009 Va
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSLPYTNAGKSTFGDGTTLTVKP

SEQ ID NO: 66 - protein, 133 aa, Artificial Sequence
  Synthetic sequence TCR1009 Vb
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSDRQGSNQPQHFGDGTRLSIL

SEQ ID NO: 67 - protein, 134 aa, Artificial Sequence
  Synthetic sequence TCR1012 Va
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDDGGGGNKLTFGTGTQLKVEL

SEQ ID NO: 68 - protein, 134 aa, Artificial Sequence
  Synthetic sequence TCR1012 Vb
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSSGGPGYEQYFGPGTRLTVT

SEQ ID NO: 69 - protein, 136 aa, Artificial Sequence
  Synthetic sequence TCR1016 Va
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRERGGGYQKVTFGIGTKLQVIP

SEQ ID NO: 70 - protein, 144 aa, Artificial Sequence
  Synthetic sequence TCR1016 Vb
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLRTGEYNEQFFGPGTRLTVL

SEQ ID NO: 71 - protein, 135 aa, Artificial Sequence
  Synthetic sequence TCR1021 Va
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYLLAGGPDNEQFFGPGTRLTVL

SEQ ID NO: 72 - protein, 144 aa, Artificial Sequence; identical to SEQ ID NO: 70
  Synthetic sequence TCR1021 Vb

SEQ ID NO: 73 - protein, 132 aa, Artificial Sequence
  Synthetic sequence TCR1027 Va
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVDENTGNQFYFGTGTSLTVIP

SEQ ID NO: 74 - protein, 131 aa, Artificial Sequence
  Synthetic sequence TCR1027 Vb
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASRIGTSQEQYFGPGTRLTVT

SEQ ID NO: 75 - protein, 127 aa, Artificial Sequence
  Synthetic sequence TCR1034 Va
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCLPSNDYKLSFGAGTTVTVRA

SEQ ID NO: 76 - protein, 134 aa, Artificial Sequence
  Synthetic sequence TCR1034 Vb
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASYRDTSSYNEQFFGPGTRLTVL

SEQ ID NO: 77 - protein, 130 aa, Artificial Sequence
  Synthetic sequence TCR1042 Va
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRYGNNRLAFGKGNQVVVIP

SEQ ID NO: 78 - protein, 134 aa, Artificial Sequence
  Synthetic sequence TCR1042 Vb
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQEWRRLADEQFFGPGTRLTVL

SEQ ID NO: 79 - protein, 131 aa, Artificial Sequence
  Synthetic sequence TCR1051 Va
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVSSGTYKYIFGTGTRLKVLA

SEQ ID NO: 80 - protein, 133 aa, Artificial Sequence
  Synthetic sequence TCR1051 Vb
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPGTGGNTEAFFGQGTRLTVV

SEQ ID NO: 81 - protein, 134 aa, Artificial Sequence
  Synthetic sequence TCR1061 Va
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAIMTGTASKLTFGKGTLLTVNP

SEQ ID NO: 82 - protein, 133 aa, Artificial Sequence
  Synthetic sequence TCR1061 Vb
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSPSGANVLTFGAGSRLTVL

SEQ ID NO: 83 - protein, 131 aa, Artificial Sequence
  Synthetic sequence TCR1072 Va
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTGTGGFKTIFGAGTRLFVKA

SEQ ID NO: 84 - protein, 137 aa, Artificial Sequence
  Synthetic sequence TCR1072 Vb
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSSMSIAAGNTGELFFGEGSRLTVL

SEQ ID NO: 85 - protein, 141 aa, Artificial Sequence
  Synthetic sequence Ca
DIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 177 aa, Artificial Sequence
  Synthetic sequence Cb 1
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 87 - protein, 178 aa, Artificial Sequence
  Synthetic sequence Cb 2
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 88 - DNA, 36 bp, Artificial Sequence
  Synthetic sequence TCR1007 CDR3a
tgtgcagtcccgaacaccggtaaccagttctatttt

SEQ ID NO: 89 - DNA, 21 bp, Artificial Sequence
  Synthetic sequence TCR1007 CDR2a
atacgtccagatgtgagtgaa

SEQ ID NO: 90 - DNA, 18 bp, Artificial Sequence
  Synthetic sequence TCR1007 CDR1a
aacactgcgtttgactac

SEQ ID NO: 91 - DNA, 45 bp, Artificial Sequence
  Synthetic sequence TCR1007 CDR3b
tgtgccagcagcttaatagcggggctctcctacgagcagtacttc

SEQ ID NO: 92 - DNA, 15 bp, Artificial Sequence
  Synthetic sequence TCR1007 CDR2b
tcgggtcatgtatcc

SEQ ID NO: 93 - DNA, 18 bp, Artificial Sequence
  Synthetic sequence TCR1007 CDR1b
ttccagaatgaagctcaa

SEQ ID NO: 94 - DNA, 45 bp, Artificial Sequence
  Synthetic sequence TCR1009 CDR3a
tgtgctctgagtcttccttacaccaatgcaggcaaatcaaccttt

SEQ ID NO: 95 - DNA, 24 bp, Artificial Sequence
  Synthetic sequence TCR1009 CDR2a
cggaactcttttgatgagcaaaat

SEQ ID NO: 96 - DNA, 21 bp, Artificial Sequence
  Synthetic sequence TCR1009 CDR1a
acccgtgatactacttattac

SEQ ID NO: 97 - DNA, 45 bp, Artificial Sequence
  Synthetic sequence TCR1009 CDR3b
tgtgccagcagcgaccgccaggggtccaatcagccccagcatttt

SEQ ID NO: 98 - DNA, 15 bp, Artificial Sequence
  Synthetic sequence TCR1009 CDR2b
atgaaccataactcc

SEQ ID NO: 99 - DNA, 18 bp, Artificial Sequence
  Synthetic sequence TCR1009 CDR1b
tcagcttctgagggtacc

SEQ ID NO: 100 - DNA, 45 bp, Artificial Sequence
  Synthetic sequence TCR1012 CDR3a
tgtgctgtgagagacgacgggggaggaggaaacaaactcaccttt

SEQ ID NO: 101 - DNA, 24 bp, Artificial Sequence
  Synthetic sequence TCR1012 CDR2a
tacatcacaggggataacctggtt

SEQ ID NO: 102 - DNA, 18 bp, Artificial Sequence
  Synthetic sequence TCR1012 CDR1a
gtctctggaaacccttat

SEQ ID NO: 103 - DNA, 45 bp, Artificial Sequence
  Synthetic sequence TCR1012 CDR3b
tgtgccagcagctcctcaggggggcctgggtacgagcagtacttc

SEQ ID NO: 104 - DNA, 18 bp, Artificial Sequence
  Synthetic sequence TCR1012 CDR2b
ttccagaatgaagctcaa

SEQ ID NO: 105 - DNA, 15 bp, Artificial Sequence
  Synthetic sequence TCR1012 CDR1b
tcgggtcatgtatcc

SEQ ID NO: 106 - DNA, 45 bp, Artificial Sequence
  Synthetic sequence TCR1016 CDR3a
tgtgcaatgagagagcgtggtgggggttaccagaaagttaccttt

SEQ ID NO: 107 - DNA, 24 bp, Artificial Sequence
  Synthetic sequence TCR1016 CDR2a
caggggtcttatgaccagcaaaat

SEQ ID NO: 108 - DNA, 21 bp, Artificial Sequence
  Synthetic sequence TCR1016 CDR1a
accagtgatccaagttatggt

SEQ ID NO: 109 - DNA, 48 bp, Artificial Sequence
  Synthetic sequence TCR1016 CDR3b
tgtgccagcagcttattgcggacaggggaatacaatgagcagttcttc

SEQ ID NO: 110 - DNA, 18 bp, Artificial Sequence
  Synthetic sequence TCR1016 CDR2b
ttttatgaaaagatgcag

SEQ ID NO: 111 - DNA, 15 bp, Artificial Sequence
  Synthetic sequence TCR1016 CDR1b
cctagacacgacact

SEQ ID NO: 112 - DNA, 45 bp, Artificial Sequence
  Synthetic sequence TCR1021 CDR3a
tgtgttgtgagtgatggggacagcagtgcttccaagataatcttt

SEQ ID NO: 113 - DNA, 24 bp, Artificial Sequence
  Synthetic sequence TCR1021 CDR2a
tacacatcagcggccaccctggtt

SEQ ID NO: 114 - DNA, 18 bp, Artificial Sequence
  Synthetic sequence TCR1021 CDR1a
tcttcttattcaccatct

SEQ ID NO: 115 - DNA, 51 bp, Artificial Sequence
  Synthetic sequence TCR1021 CDR3b
tgtgccagcagttacttactagcgggagggcctgataatgagcagttcttc

SEQ ID NO: 116 - DNA, 18 bp, Artificial Sequence
  Synthetic sequence TCR1021 CDR2b
tcagttggtgctggtatc

SEQ ID NO: 117 - DNA, 15 bp, Artificial Sequence
  Synthetic sequence TCR1021 CDR1b
atgaaccataactac

SEQ ID NO: 118 - DNA, 39 bp, Artificial Sequence
  Synthetic sequence TCR1027 CDR3a
tgtgccgtcgatgaaaacaccggtaaccagttctatttt

SEQ ID NO: 119 - DNA, 18 bp, Artificial Sequence
  Synthetic sequence TCR1027 CDR2a
atatactccaatggtgac

SEQ ID NO: 120 - DNA, 18 bp, Artificial Sequence
  Synthetic sequence TCR1027 CDR1a
gaccgaggttcccagtcc

SEQ ID NO: 121 - DNA, 39 bp, Artificial Sequence
  Synthetic sequence TCR1027 CDR3b
tgtgccagcaggatcgggacttcccaagagcagtacttc

SEQ ID NO: 122 - DNA, 18 bp, Artificial Sequence
  Synthetic sequence TCR1027 CDR2b
tattatgagaaagaagag

SEQ ID NO: 123 - DNA, 15 bp, Artificial Sequence
  Synthetic sequence TCR1027 CDR1b
tctgggcacaagagt

SEQ ID NO: 124 - DNA, 33 bp, Artificial Sequence
  Synthetic sequence TCR1034 CDR3a
tgcctgccttctaacgactacaagctcagcttt

SEQ ID NO: 125 - DNA, 21 bp, Artificial Sequence
  Synthetic sequence TCR1034 CDR2a
ttagtgaagagtggagaagtg

SEQ ID NO: 126 - DNA, 15 bp, Artificial Sequence
  Synthetic sequence TCR1034 CDR1a
actactttaagcaat

SEQ ID NO: 127 - DNA, 45 bp, Artificial Sequence
  Synthetic sequence TCR1034 CDR3b
tgtgccagctaccgggacaccagctcctacaatgagcagttcttc

SEQ ID NO: 128 - DNA, 18 bp, Artificial Sequence
  Synthetic sequence TCR1034 CDR2b
tttaacaacaacgttccg

SEQ ID NO: 129 - DNA, 15 bp, Artificial Sequence
  Synthetic sequence TCR1034 CDR1b
tcaggccacaactcc

SEQ ID NO: 130 - DNA, 36 bp, Artificial Sequence
  Synthetic sequence TCR1042 CDR3a
tgtgctctccgctatgggaacaacagactcgctttt

SEQ ID NO: 131 - DNA, 21 bp, Artificial Sequence
  Synthetic sequence TCR1042 CDR2a
gccacgaaggctgatgacaag

SEQ ID NO: 132 - DNA, 18 bp, Artificial Sequence
  Synthetic sequence TCR1042 CDR1a
gccacaggatacccttcc

SEQ ID NO: 133 - DNA, 48 bp, Artificial Sequence
  Synthetic sequence TCR1042 CDR3b
tgtgccagcagccaagaatggcgacgactagccgatgagcagttcttc

SEQ ID NO: 134 - DNA, 18 bp, Artificial Sequence
  Synthetic sequence TCR1042 CDR2b
tacaataataaggagctc

SEQ ID NO: 135 - DNA, 15 bp, Artificial Sequence
  Synthetic sequence TCR1042 CDR1b
ctgggccatgatact

SEQ ID NO: 136 - DNA, 36 bp, Artificial Sequence
  Synthetic sequence TCR1051 CDR3a
tgtgctgtgagctcaggaacctacaaatacatcttt

SEQ ID NO: 137 - DNA, 24 bp, Artificial Sequence
  Synthetic sequence TCR1051 CDR2a
tatttatcaggatccaccctggtt

SEQ ID NO: 138 - DNA, 18 bp, Artificial Sequence
  Synthetic sequence TCR1051 CDR1a
tcgtctgtttcagtgtat

SEQ ID NO: 139 - DNA, 45 bp, Artificial Sequence
  Synthetic sequence TCR1051 CDR3b
tgcgccagcagccccggtacaggagggaacactgaagctttcttt

SEQ ID NO: 140 - DNA, 18 bp, Artificial Sequence
  Synthetic sequence TCR1051 CDR2b
tacttcagtgagacacag

SEQ ID NO: 141 - DNA, 15 bp, Artificial Sequence
  Synthetic sequence TCR1051 CDR1b
tctgggcataggagt

SEQ ID NO: 142 - DNA, 39 bp, Artificial Sequence
  Synthetic sequence TCR1061 CDR3a
tgtgcaattatgaccggcactgccagtaaactcaccttt

SEQ ID NO: 143 - DNA, 24 bp, Artificial Sequence
  Synthetic sequence TCR1061 CDR2a
caagaagcttataagcaacagaat

SEQ ID NO: 144 - DNA, 21 bp, Artificial Sequence
  Synthetic sequence TCR1061 CDR1a
accagtgagagtgattattat

SEQ ID NO: 145 - DNA, 42 bp, Artificial Sequence
  Synthetic sequence TCR1061 CDR3b
tgtgccagcagctccccctctggggccaacgtcctgactttc

SEQ ID NO: 146 - DNA, 15 bp, Artificial Sequence
  Synthetic sequence TCR1061 CDR2b
tcgggtcatgtatcc

SEQ ID NO: 147 - DNA, 18 bp, Artificial Sequence
  Synthetic sequence TCR1061 CDR1b
ttccagaatgaagctcaa

SEQ ID NO: 148 - DNA, 36 bp, Artificial Sequence
  Synthetic sequence TCR1007 CDR3a
tgtgccgtgcctaacaccggcaaccagttctacttt

SEQ ID NO: 149 - DNA, 21 bp, Artificial Sequence
  Synthetic sequence TCR1007 CDR2a
attagacccgacgtgtccgag

SEQ ID NO: 150 - DNA, 18 bp, Artificial Sequence
  Synthetic sequence TCR1007 CDR1a
aataccgccttcgactac

SEQ ID NO: 151 - DNA, 45 bp, Artificial Sequence
  Synthetic sequence TCR1007 CDR3b
tgtgcctcttctctgatcgccggcctgagctacgagcagtatttt

SEQ ID NO: 152 - DNA, 18 bp, Artificial Sequence
  Synthetic sequence TCR1007 CDR2b
tttcagaatgaggcccag

SEQ ID NO: 153 - DNA, 15 bp, Artificial Sequence
  Synthetic sequence TCR1007 CDR1b
agcggccacgtgtcc

SEQ ID NO: 154 - DNA, 45 bp, Artificial Sequence
  Synthetic sequence TCR1009 CDR3a
tgcgccctgagcctgccttacaccaacgccggcaagagcaccttc

SEQ ID NO: 155 - DNA, 24 bp, Artificial Sequence
  Synthetic sequence TCR1009 CDR2a
cggaacagcttcgacgagcagaac

SEQ ID NO: 156 - DNA, 21 bp, Artificial Sequence
  Synthetic sequence TCR1009 CDR1a
acacgggacaccacctactac

SEQ ID NO: 157 - DNA, 45 bp, Artificial Sequence
  Synthetic sequence TCR1009 CDR3b
tgtgccagcagcgatcggcagggcagcaatcagcctcagcatttt

SEQ ID NO: 158 - DNA, 18 bp, Artificial Sequence
  Synthetic sequence TCR1009 CDR2b
tctgccagcgagggcacc

SEQ ID NO: 159 - DNA, 15 bp, Artificial Sequence
  Synthetic sequence TCR1009 CDR1b
atgaaccacaacagc

SEQ ID NO: 160 - DNA, 45 bp, Artificial Sequence
  Synthetic sequence TCR1012 CDR3a
tgcgctgtcagagatgatggcggcggaggcaacaagctgaccttt

SEQ ID NO: 161 - DNA, 24 bp, Artificial Sequence
  Synthetic sequence TCR1012 CDR2a
tacatcaccggcgacaacctggtc

SEQ ID NO: 162 - DNA, 18 bp, Artificial Sequence
  Synthetic sequence TCR1012 CDR1a
gtgtccggcaatccctat

SEQ ID NO: 163 - DNA, 45 bp, Artificial Sequence
  Synthetic sequence TCR1012 CDR3b
tgcgccagctcatcttctggcggccctggatatgagcagtacttc

SEQ ID NO: 164 - DNA, 18 bp, Artificial Sequence
  Synthetic sequence TCR1012 CDR2b
ttccagaacgaagctcag

SEQ ID NO: 165 - DNA, 15 bp, Artificial Sequence
  Synthetic sequence TCR1012 CDR1b
tccggccatgtctca

SEQ ID NO: 166 - DNA, 45 bp, Artificial Sequence
  Synthetic sequence TCR1016 CDR3a
tgcgccatgagagagagaggcggcggataccagaaagtgaccttt

SEQ ID NO: 167 - DNA, 24 bp, Artificial Sequence
  Synthetic sequence TCR1016 CDR2a
cagggcagctacgaccagcagaat

SEQ ID NO: 168 - DNA, 21 bp, Artificial Sequence
  Synthetic sequence TCR1016 CDR1a
accagcgatcctagctacggc

SEQ ID NO: 169 - DNA, 48 bp, Artificial Sequence
  Synthetic sequence TCR1016 CDR3b
tgtgccagctctctgctgagaaccggcgagtacaacgagcagttcttc

SEQ ID NO: 170 - DNA, 18 bp, Artificial Sequence
  Synthetic sequence TCR1016 CDR2b
ttctacgagaagatgcag

SEQ ID NO: 171 - DNA, 15 bp, Artificial Sequence
  Synthetic sequence TCR1016 CDR1b
ccacggcacgacacc

SEQ ID NO: 172 - DNA, 45 bp, Artificial Sequence
  Synthetic sequence TCR1021 CDR3a
tgcgtggtgtccgatggcgatagcagcgccagcaagatcattttc

SEQ ID NO: 173 - DNA, 24 bp, Artificial Sequence
  Synthetic sequence TCR1021 CDR2a
tacacctctgccgccacactggtc

SEQ ID NO: 174 - DNA, 18 bp, Artificial Sequence
  Synthetic sequence TCR1021 CDR1a
agcagctacagcccctct

SEQ ID NO: 175 - DNA, 51 bp, Artificial Sequence
  Synthetic sequence TCR1021 CDR3b
tgtgctagcagctacctgctggctggcggccctgataatgagcagtttttt

SEQ ID NO: 176 - DNA, 18 bp, Artificial Sequence
  Synthetic sequence TCR1021 CDR2b
tctgtcggagccggcatc

SEQ ID NO: 177 - DNA, 15 bp, Artificial Sequence
  Synthetic sequence TCR1021 CDR1b
atgaaccacaactac

SEQ ID NO: 178 - DNA, 39 bp, Artificial Sequence
  Synthetic sequence TCR1027 CDR3a
tgcgccgtggatgagaacaccggcaaccagttctacttc

SEQ ID NO: 179 - DNA, 18 bp, Artificial Sequence
  Synthetic sequence TCR1027 CDR2a
atctacagcaacggcgac

SEQ ID NO: 180 - DNA, 18 bp, Artificial Sequence
  Synthetic sequence TCR1027 CDR1a
gacagaggcagccagagc

SEQ ID NO: 181 - DNA, 39 bp, Artificial Sequence
  Synthetic sequence TCR1027 CDR3b
tgcgcctctagaatcggcacaagccaagagcagtacttt

SEQ ID NO: 182 - DNA, 18 bp, Artificial Sequence
  Synthetic sequence TCR1027 CDR2b
tactacgagaaagaggaa

SEQ ID NO: 183 - DNA, 15 bp, Artificial Sequence
  Synthetic sequence TCR1027 CDR1b
tccggccacaagagc

SEQ ID NO: 184 - DNA, 33 bp, Artificial Sequence
  Synthetic sequence TCR1034 CDR3a
tgcctgcctagcaacgactacaagctgagcttt

SEQ ID NO: 185 - DNA, 21 bp, Artificial Sequence
  Synthetic sequence TCR1034 CDR2a
ctggtcaagtccggcgaagtg

SEQ ID NO: 186 - DNA, 15 bp, Artificial Sequence
  Synthetic sequence TCR1034 CDR1a
accacactgagcaac

SEQ ID NO: 187 - DNA, 45 bp, Artificial Sequence
  Synthetic sequence TCR1034 CDR3b
tgcgccagctacagagacaccagcagctacaacgagcagttcttc

SEQ ID NO: 188 - DNA, 18 bp, Artificial Sequence
  Synthetic sequence TCR1034 CDR2b
ttcaacaacaacgtgccc

SEQ ID NO: 189 - DNA, 15 bp, Artificial Sequence
  Synthetic sequence TCR1034 CDR1b
agcggccacaatagc

SEQ ID NO: 190 - DNA, 36 bp, Artificial Sequence
  Synthetic sequence TCR1042 CDR3a
tgtgccctgagatacggcaacaaccggctggccttt

SEQ ID NO: 191 - DNA, 18 bp, Artificial Sequence
  Synthetic sequence TCR1042 CDR2a
gccacaggctaccccagt

SEQ ID NO: 192 - DNA, 21 bp, Artificial Sequence
  Synthetic sequence TCR1042 CDR1a
gccacaaaggccgacgacaag

SEQ ID NO: 193 - DNA, 48 bp, Artificial Sequence
  Synthetic sequence TCR1042 CDR3b
tgtgccagcagccaagagtggcggagactggccgatgagcagtttttt

SEQ ID NO: 194 - DNA, 15 bp, Artificial Sequence
  Synthetic sequence TCR1042 CDR2b
ctgggccacgacacc

SEQ ID NO: 195 - DNA, 18 bp, Artificial Sequence
  Synthetic sequence TCR1042 CDR1b
tacaacaacaaagagctg

SEQ ID NO: 196 - DNA, 36 bp, Artificial Sequence
  Synthetic sequence TCR1051 CDR3a
tgtgccgtgtccagcggcacctacaagtacatcttt

SEQ ID NO: 197 - DNA, 24 bp, Artificial Sequence
  Synthetic sequence TCR1051 CDR2a
tatctgagcggcagcacactggtg

SEQ ID NO: 198 - DNA, 18 bp, Artificial Sequence
  Synthetic sequence TCR1051 CDR1a
agctccgtgtccgtgtac

SEQ ID NO: 199 - DNA, 45 bp, Artificial Sequence
  Synthetic sequence TCR1051 CDR3b
tgcgcttctagtcctggcacaggcggcaataccgaggcctttttt

SEQ ID NO: 200 - DNA, 18 bp, Artificial Sequence
  Synthetic sequence TCR1051 CDR2b
tacttcagcgagacacag

SEQ ID NO: 201 - DNA, 15 bp, Artificial Sequence
  Synthetic sequence TCR1051 CDR1b
tccggccacagaagc

SEQ ID NO: 202 - DNA, 39 bp, Artificial Sequence
  Synthetic sequence TCR1061 CDR3a
tgtgccatcatgaccggcaccgccagcaagctgacattt

SEQ ID NO: 203 - DNA, 24 bp, Artificial Sequence
  Synthetic sequence TCR1061 CDR2a
caagaggcctataagcagcagaac

SEQ ID NO: 204 - DNA, 21 bp, Artificial Sequence
  Synthetic sequence TCR1061 CDR1a
accagcgagagcgactactac

SEQ ID NO: 205 - DNA, 42 bp, Artificial Sequence
  Synthetic sequence TCR1061 CDR3b
tgtgccagcagttctccttctggcgccaacgtgctgaccttt

SEQ ID NO: 206 - DNA, 18 bp, Artificial Sequence
  Synthetic sequence TCR1061 CDR2b
tttcagaatgaggctcag

SEQ ID NO: 207 - DNA, 15 bp, Artificial Sequence
  Synthetic sequence TCR1061 CDR1b
agcggccacgtgtcc

SEQ ID NO: 208 - DNA, 417 bp, Artificial Sequence
  Synthetic sequence TCR1007 Va
atggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagtcccgaacaccggtaaccagttctattttgggacagggacaagtttgacggtcattcca

SEQ ID NO: 209 - DNA, 402 bp, Artificial Sequence
  Synthetic sequence TCR1007 Vb
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagcttaatagcggggctctcctacgagcagtacttcgggccgggcaccaggctcacggtcaca

SEQ ID NO: 210 - DNA, 408 bp, Artificial Sequence
  Synthetic sequence TCR1009 Va
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtcttccttacaccaatgcaggcaaatcaacctttggggatgggactacgctcactgtgaagcca

SEQ ID NO: 211 - DNA, 399 bp, Artificial Sequence
  Synthetic seqeunce TCR1009 Vb
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagcgaccgccaggggtccaatcagccccagcattttggtgatgggactcgactctccatcctg

SEQ ID NO: 212 - DNA, 402 bp, Artificial Sequence
  Synthetic seqeunce TCR1012 Va
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacgacgggggaggaggaaacaaactcacctttgggacaggcactcagctaaaagtggaactc

SEQ ID NO: 213 - DNA, 402 bp, Artificial Sequence
  Synthetic seqeunce TCR1012 Vb
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagctcctcaggggggcctgggtacgagcagtacttcgggccgggcaccaggctcacggtcaca

SEQ ID NO: 214 - DNA, 408 bp, Artificial Sequence
  Synthetic seqeunce TCR1016 Va
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagagcgtggtgggggttaccagaaagttacctttggaattggaacaaagctccaagtcatccca

SEQ ID NO: 215 - DNA, 432 bp, Artificial Sequence
  Synthetic seqeunce TCR1016 Vb
atgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttattgcggacaggggaatacaatgagcagttcttcgggccagggacacggctcaccgtgcta

SEQ ID NO: 216 - DNA, 402 bp, Artificial Sequence
  Synthetic seqeunce TCR1021 Va
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgagtgatggggacagcagtgcttccaagataatctttggatcagggaccagactcagcatccggcca

SEQ ID NO: 217 - DNA, 405 bp, Artificial Sequence
  Synthetic seqeunce TCR1021 Vb
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttacttactagcgggagggcctgataatgagcagttcttcgggccagggacacggctcaccgtgcta

SEQ ID NO: 218 - DNA, 396 bp, Artificial Sequence
  Synthetic seqeunce TCR1027 Va
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtcgatgaaaacaccggtaaccagttctattttgggacagggacaagtttgacggtcattcca

SEQ ID NO: 219 - DNA, 393 bp, Artificial Sequence
  Synthetic seqeunce TCR1027 Vb
atgggccctgggctcctctgctgggtgctgctttgtctcctgggagcaggcccagtggacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcaggatcgggacttcccaagagcagtacttcgggccgggcaccaggctcacggtcaca

SEQ ID NO: 220 - DNA, 381 bp, Artificial Sequence
  Synthetic seqeunce TCR1034 Va
atgctactcatcacatcaatgttggtcttatggatgcaattgtcacaggtgaatggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgcctgccttctaacgactacaagctcagctttggagccggaaccacagtaactgtaagagca

SEQ ID NO: 221 - DNA, 402 bp, Artificial Sequence
  Synthetic seqeunce TCR1034 Vb
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagctaccgggacaccagctcctacaatgagcagttcttcgggccagggacacggctcaccgtgcta

SEQ ID NO: 222 - DNA, 390 bp, Artificial Sequence
  Synthetic seqeunce TCR1042 Va
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctccgctatgggaacaacagactcgcttttgggaaggggaaccaagtggtggtcatacca

SEQ ID NO: 223 - DNA, 402 bp, Artificial Sequence
  Synthetic seqeunce TCR1042 Vb
atgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagaatggcgacgactagccgatgagcagttcttcgggccagggacacggctcaccgtgcta

SEQ ID NO: 224 - DNA, 393 bp, Artificial Sequence
  Synthetic seqeunce TCR1051 Va
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgagctcaggaacctacaaatacatctttggaacaggcaccaggctgaaggttttagca

SEQ ID NO: 225 - DNA, 399 bp, Artificial Sequence
  Synthetic seqeunce TCR1051 Vb
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccccggtacaggagggaacactgaagctttctttggacaaggcaccagactcacagttgtg

SEQ ID NO: 226 - DNA, 402 bp, Artificial Sequence
  Synthetic seqeunce TCR1061 Va
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcaattatgaccggcactgccagtaaactcacctttggaaaaggaactctcttaaccgtgaatcca

SEQ ID NO: 227 - DNA, 399 bp, Artificial Sequence
  Synthetic seqeunce TCR1061 Vb
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagctccccctctggggccaacgtcctgactttcggggccggcagcaggctgaccgtgctg

SEQ ID NO: 228 - DNA, 417 bp, Artificial Sequence
  Synthetic seqeunce TCR1007 Va
atggacaagatcctgggcgccagctttctggtgctgtggctgcaactgtgttgggtgtccggccagcagaaagagaagtccgaccagcagcaagtgaaacagagccctcagagcctgatcgtgcagaaaggcggcatcagcatcatcaactgcgcctacgagaataccgccttcgactacttcccctggtatcagcagttccccggcaagggacctgctctgctgatcgccattagacccgacgtgtccgagaagaaagagggcagattcaccatcagcttcaacaagagcgccaagcagttcagcctgcacatcatggatagccagcctggcgacagcgccacctacttttgtgccgtgcctaacaccggcaaccagttctactttggcaccggcaccagcctgacagtgatccct

SEQ ID NO: 229 - DNA, 402 bp, Artificial Sequence
  Synthetic seqeunce TCR1007 Vb
atgggcaccagactgctgtgctgggtcgtgctgggatttctgggcacagatcatacaggcgccggtgtcagccagtctcctagatacaaggtggccaagcgcggacaggatgtggccctgagatgtgatcctatcagcggccacgtgtccctgttctggtatcaacaggccctcggacagggccccgagttcctgacctactttcagaatgaggcccagctggacaagagcggcctgcctagcgatagattcttcgccgaaagacccgagggcagcgtgtccacactgaagatccagagaacccagcaagaggacagcgccgtgtacctgtgtgcctcttctctgatcgccggcctgagctacgagcagtattttggccctggcacacggctgaccgtgacc

SEQ ID NO: 230 - DNA, 408 bp, Artificial Sequence
  Synthetic seqeunce TCR1009 Va
atgcttacagctagcctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttttggtacaagcagcctcctagcggcgagctggtgttcctgatcagacggaacagcttcgacgagcagaacgagatctccggccggtacagctggaacttccagaagtccacctccagcttcaatttcacaatcaccgccagccaggtggtggactctgccgtgtatttctgcgccctgagcctgccttacaccaacgccggcaagagcaccttcggagatggcacaaccctgactgtgaagccc

SEQ ID NO: 231 - DNA, 399 bp, Artificial Sequence
  Synthetic seqeunce TCR1009 Vb
atgagcatcggcctgctgtgttgcgtggcattctctctgctgtgggcctctcctgtgaatgccggcgtgacacagacccctaagttccaggtgctgaaaaccggccagagcatgaccctgcagtgtgcccaggacatgaaccacaacagcatgtactggtacagacaggaccccggcatgggcctgagactgatctactactctgccagcgagggcaccaccgacaaaggcgaagtgcccaatggctacaacgtgtcccggctgaacaagagagagttctccctgcggctggaaagcgccgctccttctcagacctccgtgtacttctgtgccagcagcgatcggcagggcagcaatcagcctcagcattttggcgacggcacccggctgagcattctg

SEQ ID NO: 232 - DNA, 402 bp, Artificial Sequence
  Synthetic seqeunce TCR1012 Va
atggcatctgcccctatctccatgctggccatgctgtttaccctgtctggcctgagagcccagtctgtggctcagcctgaggaccaagtgaatgtggccgagggcaatcccctgaccgtcaagtgtacctactccgtgtccggcaatccctatctcttttggtacgtgcagtaccccaaccggggcctgcagttcctgctgaagtacatcaccggcgacaacctggtcaagggcagctatggattcgaggccgagttcaacaagtcccagaccagcttccacctgaagaaacccagcgctctggtgtccgatagcgccctgtatttttgcgctgtcagagatgatggcggcggaggcaacaagctgacctttggaactggcacccagctgaaggtggaactg

SEQ ID NO: 233 - DNA, 402 bp, Artificial Sequence
  Synthetic seqeunce TCR1012 Vb
atgggaacacgcctgctctgttgggttgtgctcggcttcctgggaaccgatcacactggtgccggtgtttctcagagcccacggtacaaagtggctaagagaggccaggacgtcgcactgagatgcgaccctatttccggccatgtctcacttttttggtatcagcaagctctcggccaggggcctgaatttctgacatatttccagaacgaagctcagctcgacaagtccgggctgccctccgacagattttttgccgaaaggcctgaaggctccgtgtctaccctgaaaattcagcggacacaacaagaggactccgctgtctatctgtgcgccagctcatcttctggcggccctggatatgagcagtacttcggaccaggcactagactcaccgtgacc

SEQ ID NO: 234 - DNA, 408 bp, Artificial Sequence
  Synthetic seqeunce TCR1016 Va
atgagcctgagcagcctgctgaaggtcgtgacagcctctctgtggctcggacctggaatcgcccagaagatcacccagacacagcccggcatgttcgtgcaagagaaagaagccgtgacactggactgcacctacgacaccagcgatcctagctacggcctgttctggtacaagcagcctagcagcggcgagatgatcttcctgatctaccagggcagctacgaccagcagaatgccaccgagggcagatacagcctgaacttccagaaggcccggaagtccgccaacctggtcatttctgctagccagctgggcgacagcgccatgtacttttgcgccatgagagagagaggcggcggataccagaaagtgacctttggcatcggcaccaagctgcaagtgatcccc

SEQ ID NO: 235 - DNA, 432 bp, Artificial Sequence
  Synthetic seqeunce TCR1016 Vb
atgctgagccccgacctgcctgattctgcctggaataccagactgctgtgccacgtgatgctgtgcctgctgggagctgtttctgtggccgctggcgttatccagtctcctcggcacctgatcaaagagaagagagagacagccacactgaagtgctaccccattccacggcacgacaccgtgtactggtatcagcaaggcccaggccaggatcctcagttcctgatcagcttctacgagaagatgcagagcgacaagggcagcatccccgacagattttctgcccagcagttcagcgactaccacagcgagctgaacatgtccagcctggaactgggagatagcgccctgtatttctgtgccagctctctgctgagaaccggcgagtacaacgagcagttcttcggccctggcaccagactgacagtgctg

SEQ ID NO: 236 - DNA, 402 bp, Artificial Sequence
  Synthetic seqeunce TCR1021 Va
atgctgctgcttctggtgcccgtgctggaagtgatctttaccctcggcggaacaagagcccagagcgtgacacagctggatagccacgtgtccgtgtctgagggaacacccgtgctgctgagatgcaactacagcagcagctacagcccctctctgttttggtacgtgcagcaccccaacaagggcctgcaactgctgctgaagtacacctctgccgccacactggtcaagggcatcaatggcttcgaggccgagttcaagaagtccgagacaagcttccacctgaccaagcctagcgctcacatgtctgatgccgccgagtacttctgcgtggtgtccgatggcgatagcagcgccagcaagatcattttcggcagcggcacccggctgagcatcagacct

SEQ ID NO: 237 - DNA, 405 bp, Artificial Sequence
  Synthetic seqeunce TCR1021 Vb
atgagcatctccctgctgtgctgcgccgcttttcctctgctttgggccggacctgtgaatgccggcgttacacagacccctaagttccggatcctgaagatcggccagagcatgaccctgcagtgcacccaggacatgaaccacaactacatgtattggtacagacaggaccccggcatgggcctgaaactgatctactactctgtcggagccggcatcaccgacaaaggcgaagtgcccaatggctacaacgtgtccagaagcaccaccgaggacttccctctgcggctggaacttgctgccccatctcagaccagcgtgtacttctgtgctagcagctacctgctggctggcggccctgataatgagcagttttttggccccggaacacggctgaccgtcctg

SEQ ID NO: 238 - DNA, 396 bp, Artificial Sequence
  Synthetic seqeunce TCR1027 Va
atgaagtctctgagagtgctgctggtcatcctgtggctgcagctgtcttgggtctggtcccagcagaaagaggtggaacagaacagcggccctctgtctgttcctgaaggcgctatcgcctctctgaattgcacctacagcgacagaggcagccagagcttcttctggtatagacagtacagcggcaagtcccctgagctgatcatgttcatctacagcaacggcgacaaagaggacggccggtttacagcccagctgaacaaggcctctcagtacgtgtccctgctgatcagagactcccagcctagcgattccgccacctatctgtgcgccgtggatgagaacaccggcaaccagttctacttcggcaccggaaccagcctgaccgtgattcct

SEQ ID NO: 239 - DNA, 393 bp, Artificial Sequence
  Synthetic seqeunce TCR1027 Vb
atgggacctggcctgctgtgttgggtcctgctttgtctgcttggagctggccctgttgacgctggcgtcacacaatctcccacacacctgattaagaccagaggccagcaagtgaccctgaggtgctctcctatctccggccacaagagcgtcagctggtatcaacaggtgctcggccagggaccacagttcatcttccagtactacgagaaagaggaacgcggcaggggcaacttccccgatagattcagcgccagacagttccccaactactcctccgagctgaatgtgaacgccctgctgctcggagacagcgctctttacctgtgcgcctctagaatcggcacaagccaagagcagtactttggacccgggactcgcctgacagtgaca

SEQ ID NO: 240 - DNA, 381 bp, Artificial Sequence
  Synthetic seqeunce TCR1034 Va
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgcctgcctagcaacgactacaagctgagctttggcgccggaaccaccgtgacagtcagagct

SEQ ID NO: 241 - DNA, 402 bp, Artificial Sequence
  Synthetic seqeunce TCR1034 Vb
atggacagctggaccttctgctgcgtgtccctgtgtatcctggtggccaagcacacagatgccggcgtgatccagtctcctagacacgaagtgaccgagatggggcaagaagtgaccctgcgctgcaagcctatcagcggccacaatagcctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagctacagagacaccagcagctacaacgagcagttcttcggccctggcaccagactgaccgtgctg

SEQ ID NO: 242 - DNA, 390 bp, Artificial Sequence
  Synthetic seqeunce TCR1042 Va
atgaattacagccctggcctggtgtccctgatcctgctgctgctgggaagaaccagaggcaacagcgtgacccagatggaaggccctgtgactctgagcgaggaagccttcctgaccatcaactgcacctacacagccacaggctaccccagtctgttttggtacgtgcagtatcccggcgagggactgcagctgctgctcaaagccacaaaggccgacgacaagggcagcaacaagggctttgaggccacctaccggaaagagacaaccagcttccacctggaaaagggcagcgtccaggtgtccgattccgccgtgtatttttgtgccctgagatacggcaacaaccggctggcctttggcaagggcaatcaggtggtggtcatcccc

SEQ ID NO: 243 - DNA, 402 bp, Artificial Sequence
  Synthetic seqeunce TCR1042 Vb
atgggctgcagactgctgtgctgtgtggttttctgcctgctgcaagctggacccctggatacagccgtgtctcagacccctaagtacctggtcactcagatgggcaacgacaagagcatcaagtgcgagcagaacctgggccacgacaccatgtactggtataagcaggacagcaagaaattcctcaagatcatgttctcctacaacaacaaagagctgatcatcaacgagacagtgcccaaccggttcagccctaagagccctgataaggcccacctgaacctgcacattaacagcctcgagctgggcgactccgctgtctacttttgtgccagcagccaagagtggcggagactggccgatgagcagttttttggacccggcacacggctgacagtgctc

SEQ ID NO: 244 - DNA, 393 bp, Artificial Sequence
  Synthetic seqeunce TCR1051 Va
atgttgctgttgctggtgcccgccttccaagtgatctttaccctcggcggaacacgggcccagagtgtcacacagctggattctcaggtgcccgtgtttgaggaagcccctgtcgagctgagatgcaactacagcagctccgtgtccgtgtacctcttttggtatgttcagtaccccaaccagggcctgcagctcctcctgaagtatctgagcggcagcacactggtggaatccatcaatggcttcgaggccgagttcaacaagtcccagacctcattccatctgcggaagcccagcgtgcacatctctgataccgccgaatacttctgtgccgtgtccagcggcacctacaagtacatctttggcaccggcaccaggctgaaggtgctggcc

SEQ ID NO: 245 - DNA, 399 bp, Artificial Sequence
  Synthetic seqeunce TCR1051 Vb
atgggatctagactgctctgttgggtcctgctgtgtctgcttggagccggacctgtgaaagcaggcgtgacacagacacccagatacctgatcaagaccaggggccaacaagtgacactgagctgtagccctatctccggccacagaagcgtgtcctggtatcagcaaacccctggacagggcctccagttcctgttcgagtacttcagcgagacacagcggaacaagggcaacttccccggcagattttccggcagacagttcagcaactcccgcagcgagatgaacgtgtccacactggaactgggcgacagcgccctgtatctgtgcgcttctagtcctggcacaggcggcaataccgaggccttttttggccaaggcactcgcctgactgtggtg

SEQ ID NO: 246 - DNA, 402 bp, Artificial Sequence
  Synthetic seqeunce TCR1061 Va
atggcctgtcctggatttctgtgggccctcgtgatcagcacctgtctggaattcagcatggcccagaccgtgacacagagccagcctgagatgtctgtgcaagaggccgagacagtgaccctgagctgcacctacgataccagcgagagcgactactacctgttctggtacaagcagcctcctagccggcagatgatcctggtcatcagacaagaggcctataagcagcagaacgccaccgagaacagattcagcgtgaacttccagaaggccgccaagagcttcagcctgaagatcagcgatagccagctgggcgacgccgccatgtacttttgtgccatcatgaccggcaccgccagcaagctgacatttggcaagggcaccctgctgaccgtgaatccc

SEQ ID NO: 247 - DNA, 399 bp, Artificial Sequence
  Synthetic seqeunce TCR1061 Vb
atgggcacaagactgctgtgctgggtcgtgctgggctttctgggcacagatcatacaggcgccggtgtcagccagtctcctagatacaaggtggccaagcgcggacaggatgtggccctcagatgtgatcctatcagcggccacgtgtccctgttttggtatcagcaggccctcggacagggccccgagttcctgacctactttcagaatgaggctcagctggacaagagcggcctgcctagcgatagattcttcgccgaaagacccgagggcagcgtgtccacactgaagatccagagaacccagcaagaggacagcgccgtgtacctgtgtgccagcagttctccttctggcgccaacgtgctgacctttggcgctggctctagactgacagtgctg

SEQ ID NO: 248 - DNA, 393 bp, Artificial Sequence
  Synthetic seqeunce TCR1072 Va
atgatttccctgagagtgctgctcgtgattctctggctccagctctcctgggtttggagccagcggaaagaggtcgagcaagaccctgggccttttaacgttccagagggcgctacagtggcttttaattgcacatactccaacagcgcctcacagagttttttctggtatcggcaggactgtagaaaagaaccgaaactgctcatgtccgtgtatagctccggcaatgaggatggccggtttaccgctcagctgaatcgggcctctcagtacatctccctgctgattcgggactccaagctgtccgatagcgcaacatacctgtgcgtggtcacaggcaccggcggcttcaagacaatcttcggagcaggcacccggctgtttgtgaaggct

SEQ ID NO: 249 - DNA, 411 bp, Artificial Sequence
  Synthetic seqeunce TCR1072 Vb
atgagccccatctttacctgcatcaccatcctgtgcctgctggccgctggatctcctggggaagaagtggcccagacacctaagcacctcgttagaggcgagggccagaaggccaagctgtattgcgcccctatcaagggccacagctatgttttttggtatcaacaggtcctgaagaacgagttcaagttcctgatcagcttccagaacgagaacgtgttcgacgagacaggcatgcccaaagagcggttctccgccaagtgcctgcctaacagcccttgcagcctggaaatccaggccaccaagctggaagattccgccgtgtatttctgcgccagcagcagcatgtctatcgccgctggaaataccggcgagctgttcttcggcgagggcagcagactgacagttctg

SEQ ID NO: 250 - DNA, 426 bp, Artificial Sequence
  Synthetic seqeunce Ca
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 251 - DNA, 426 bp, Artificial Sequence
  Synthetic seqeunce Ca
gacatccagaaccccgaccctgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 252 - DNA, 534 bp, Artificial Sequence
  Synthetic seqeunce Cb1
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 253 - DNA, 224 bp, Artificial Sequence
  Synthetic seqeunce Cb2
atgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 254 - DNA, 66 bp, Artificial Sequence
  Synthetic seqeunce Porcine teschovirus-1 2A (P2A) peptide (nucleotide; codon-optimized)
ggttccggagccacgaacttctctctgttaaagcaagcaggagacgtggaagaaaaccccggtccc

SEQ ID NO: 255 - DNA, 66 bp, Artificial Sequence
  Synthetic seqeunce Porcine teschovirus-1 2A (P2A) peptide (nucleotide)
ggaagcggagctactaacttcagcctgctgaagcaggctggagacgtggaggagaaccctggacct

SEQ ID NO: 256 - DNA, 63 bp, Artificial Sequence
  Synthetic seqeunce Thoseaasigna virus 2A (T2A) peptide (nucleotide)
ggaagcggagagggcagaggaagtctgctaacatgcggtgacgtcgaggagaatcctggacct

SEQ ID NO: 257 - DNA, 69 bp, Artificial Sequence
  Synthetic seqeunce Equine rhinitis A virus (ERAV) 2A (E2A) peptide (nucleotide)
ggaagcggacagtgtactaattatgctctcttgaaattggctggagatgttgagagcaaccctggacct

SEQ ID NO: 258 - DNA, 75 bp, Artificial Sequence
  Synthetic seqeunce Foot-and-Mouth disease virus 2A (F2A) peptide (nucleotide)
ggaagcggagtgaaacagactttgaattttgaccttctcaagttggcgggagacgtggagtccaaccctggacct

SEQ ID NO: 259 - protein, 22 aa, Artificial Sequence
  Synthetic seqeunce  Porcine teschovirus-1 2A (P2A) peptide (amino acid)
GSGATNFSLLKQAGDVEENPGP

SEQ ID NO: 260 - protein, 24 aa, Artificial Sequence
  Synthetic seqeunce  Thoseaasigna virus 2A (T2A) peptide (amino acid)
LEGGGEGRGSLLTCGDVEENPGPR

SEQ ID NO: 261 - protein, 20 aa, Artificial Sequence
  Synthetic seqeunce  Equine rhinitis A virus (ERAV) 2A (E2A) peptide (amino acid)
QCTNYALLKLAGDVESNPGP

SEQ ID NO: 262 - protein, 25 aa, Artificial Sequence
  Synthetic seqeunce Foot-and-Mouth disease virus 2A (F2A) peptide (amino acid)
GSGVKQTLNFDLLKLAGDVESNPGP

SEQ ID NO: 263 - protein, 15 aa, Artificial Sequence
  Synthetic seqeunce Glycine-Serine linker
GGGGSGGGGSGGGGS

SEQ ID NO: 264 - protein, 18 aa, Artificial Sequence
  Synthetic seqeunce Glycine-Serine linker
GSTSGGGSGGGSGGGGSS

SEQ ID NO: 265 - DNA, 1419 bp, Artificial Sequence
  Synthetic seqeunce TCR1007 VB_CB_P2A_VA
atgggcaccagactgctgtgctgggtcgtgctgggatttctgggcacagatcatacaggcgccggtgtcagccagtctcctagatacaaggtggccaagcgcggacaggatgtggccctgagatgtgatcctatcagcggccacgtgtccctgttctggtatcaacaggccctcggacagggccccgagttcctgacctactttcagaatgaggcccagctggacaagagcggcctgcctagcgatagattcttcgccgaaagacccgagggcagcgtgtccacactgaagatccagagaacccagcaagaggacagcgccgtgtacctgtgtgcctcttctctgatcgccggcctgagctacgagcagtattttggccctggcacacggctgaccgtgaccgacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatggacaagatcctgggcgccagctttctggtgctgtggctgcaactgtgttgggtgtccggccagcagaaagagaagtccgaccagcagcaagtgaaacagagccctcagagcctgatcgtgcagaaaggcggcatcagcatcatcaactgcgcctacgagaataccgccttcgactacttcccctggtatcagcagttccccggcaagggacctgctctgctgatcgccattagacccgacgtgtccgagaagaaagagggcagattcaccatcagcttcaacaagagcgccaagcagttcagcctgcacatcatggatagccagcctggcgacagcgccacctacttttgtgccgtgcctaacaccggcaaccagttctactttggcaccggcaccagcctgacagtgatccct

SEQ ID NO: 266 - DNA, 1404 bp, Artificial Sequence
  Synthetic seqeunce TCR1009 VB_CB_P2A_VA
atgagcatcggcctgctgtgttgcgtggcattctctctgctgtgggcctctcctgtgaatgccggcgtgacacagacccctaagttccaggtgctgaaaaccggccagagcatgaccctgcagtgtgcccaggacatgaaccacaacagcatgtactggtacagacaggaccccggcatgggcctgagactgatctactactctgccagcgagggcaccaccgacaaaggcgaagtgcccaatggctacaacgtgtcccggctgaacaagagagagttctccctgcggctggaaagcgccgctccttctcagacctccgtgtacttctgtgccagcagcgatcggcagggcagcaatcagcctcagcattttggcgacggcacccggctgagcattctggaggacctgaacaaagtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccaccatcctgtacgagatcctgctgggcaaggccacactgtacgccgtgctggtgtctgccctggtgctgatggccatggtcaagcggaaggacttcggttccggagccacgaacttctctctgttaaagcaagcaggagacgtggaagaaaaccccggtcccatgcttacagctagcctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttttggtacaagcagcctcctagcggcgagctggtgttcctgatcagacggaacagcttcgacgagcagaacgagatctccggccggtacagctggaacttccagaagtccacctccagcttcaatttcacaatcaccgccagccaggtggtggactctgccgtgtatttctgcgccctgagcctgccttacaccaacgccggcaagagcaccttcggagatggcacaaccctgactgtgaagccc

SEQ ID NO: 267 - DNA, 1404 bp, Artificial Sequence
  Synthetic seqeunce TCR1012 VB_CB_P2A_VA
atgggaacacgcctgctctgttgggttgtgctcggcttcctgggaaccgatcacactggtgccggtgtttctcagagcccacggtacaaagtggctaagagaggccaggacgtcgcactgagatgcgaccctatttccggccatgtctcacttttttggtatcagcaagctctcggccaggggcctgaatttctgacatatttccagaacgaagctcagctcgacaagtccgggctgccctccgacagattttttgccgaaaggcctgaaggctccgtgtctaccctgaaaattcagcggacacaacaagaggactccgctgtctatctgtgcgccagctcatcttctggcggccctggatatgagcagtacttcggaccaggcactagactcaccgtgaccgacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatggcatctgcccctatctccatgctggccatgctgtttaccctgtctggcctgagagcccagtctgtggctcagcctgaggaccaagtgaatgtggccgagggcaatcccctgaccgtcaagtgtacctactccgtgtccggcaatccctatctcttttggtacgtgcagtaccccaaccggggcctgcagttcctgctgaagtacatcaccggcgacaacctggtcaagggcagctatggattcgaggccgagttcaacaagtcccagaccagcttccacctgaagaaacccagcgctctggtgtccgatagcgccctgtatttttgcgctgtcagagatgatggcggcggaggcaacaagctgacctttggaactggcacccagctgaaggtggaactg

SEQ ID NO: 268 - DNA, 1440 bp, Artificial Sequence
  Synthetic seqeunce TCR1016 VB_CB_P2A_VA
atgctgagccccgacctgcctgattctgcctggaataccagactgctgtgccacgtgatgctgtgcctgctgggagctgtttctgtggccgctggcgttatccagtctcctcggcacctgatcaaagagaagagagagacagccacactgaagtgctaccccattccacggcacgacaccgtgtactggtatcagcaaggcccaggccaggatcctcagttcctgatcagcttctacgagaagatgcagagcgacaagggcagcatccccgacagattttctgcccagcagttcagcgactaccacagcgagctgaacatgtccagcctggaactgggagatagcgccctgtatttctgtgccagctctctgctgagaaccggcgagtacaacgagcagttcttcggccctggcaccagactgacagtgctggacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatgagcctgagcagcctgctgaaggtcgtgacagcctctctgtggctcggacctggaatcgcccagaagatcacccagacacagcccggcatgttcgtgcaagagaaagaagccgtgacactggactgcacctacgacaccagcgatcctagctacggcctgttctggtacaagcagcctagcagcggcgagatgatcttcctgatctaccagggcagctacgaccagcagaatgccaccgagggcagatacagcctgaacttccagaaggcccggaagtccgccaacctggtcatttctgctagccagctgggcgacagcgccatgtacttttgcgccatgagagagagaggcggcggataccagaaagtgacctttggcatcggcaccaagctgcaagtgatcccc

SEQ ID NO: 269 - DNA, 1407 bp, Artificial Sequence
  Synthetic seqeunce TCR1021 VB_CB_P2A_VA
atgagcatctccctgctgtgctgcgccgcttttcctctgctttgggccggacctgtgaatgccggcgttacacagacccctaagttccggatcctgaagatcggccagagcatgaccctgcagtgcacccaggacatgaaccacaactacatgtattggtacagacaggaccccggcatgggcctgaaactgatctactactctgtcggagccggcatcaccgacaaaggcgaagtgcccaatggctacaacgtgtccagaagcaccaccgaggacttccctctgcggctggaacttgctgccccatctcagaccagcgtgtacttctgtgctagcagctacctgctggctggcggccctgataatgagcagttttttggccccggaacacggctgaccgtcctggacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatgctgctgcttctggtgcccgtgctggaagtgatctttaccctcggcggaacaagagcccagagcgtgacacagctggatagccacgtgtccgtgtctgagggaacacccgtgctgctgagatgcaactacagcagcagctacagcccctctctgttttggtacgtgcagcaccccaacaagggcctgcaactgctgctgaagtacacctctgccgccacactggtcaagggcatcaatggcttcgaggccgagttcaagaagtccgagacaagcttccacctgaccaagcctagcgctcacatgtctgatgccgccgagtacttctgcgtggtgtccgatggcgatagcagcgccagcaagatcattttcggcagcggcacccggctgagcatcagacct

SEQ ID NO: 270 - DNA, 1389 bp, Artificial Sequence
  Synthetic seqeunce TCR1027 VB_CB_P2A_VA_CA
atgggacctggcctgctgtgttgggtcctgctttgtctgcttggagctggccctgttgacgctggcgtcacacaatctcccacacacctgattaagaccagaggccagcaagtgaccctgaggtgctctcctatctccggccacaagagcgtcagctggtatcaacaggtgctcggccagggaccacagttcatcttccagtactacgagaaagaggaacgcggcaggggcaacttccccgatagattcagcgccagacagttccccaactactcctccgagctgaatgtgaacgccctgctgctcggagacagcgctctttacctgtgcgcctctagaatcggcacaagccaagagcagtactttggacccgggactcgcctgacagtgacagacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatgaagtctctgagagtgctgctggtcatcctgtggctgcagctgtcttgggtctggtcccagcagaaagaggtggaacagaacagcggccctctgtctgttcctgaaggcgctatcgcctctctgaattgcacctacagcgacagaggcagccagagcttcttctggtatagacagtacagcggcaagtcccctgagctgatcatgttcatctacagcaacggcgacaaagaggacggccggtttacagcccagctgaacaaggcctctcagtacgtgtccctgctgatcagagactcccagcctagcgattccgccacctatctgtgcgccgtggatgagaacaccggcaaccagttctacttcggcaccggaaccagcctgaccgtgattcct

SEQ ID NO: 271 - DNA, 1380 bp, Artificial Sequence
  Synthetic seqeunce TCR1034 VB_CB_P2A_VA_CA
atggacagctggaccttctgctgcgtgtccctgtgtatcctggtggccaagcacacagatgccggcgtgatccagtctcctagacacgaagtgaccgagatggggcaagaagtgaccctgcgctgcaagcctatcagcggccacaatagcctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagctacagagacaccagcagctacaacgagcagttcttcggccctggcaccagactgaccgtgctggaggacctgaacaaagtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccaccatcctgtacgagatcctgctgggcaaggccacactgtacgccgtgctggtgtctgccctggtgctgatggccatggtcaagcggaaggacttcggttccggagccacgaacttctctctgttaaagcaagcaggagacgtggaagaaaaccccggtcccatgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgcctgcctagcaacgactacaagctgagctttggcgccggaaccaccgtgacagtcagagct

SEQ ID NO: 272 - DNA, 1392 bp, Artificial Sequence
  Synthetic seqeunce TCR1042 VB_CB_P2A_VA
atgggctgcagactgctgtgctgtgtggttttctgcctgctgcaagctggacccctggatacagccgtgtctcagacccctaagtacctggtcactcagatgggcaacgacaagagcatcaagtgcgagcagaacctgggccacgacaccatgtactggtataagcaggacagcaagaaattcctcaagatcatgttctcctacaacaacaaagagctgatcatcaacgagacagtgcccaaccggttcagccctaagagccctgataaggcccacctgaacctgcacattaacagcctcgagctgggcgactccgctgtctacttttgtgccagcagccaagagtggcggagactggccgatgagcagttttttggacccggcacacggctgacagtgctcgacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatgaattacagccctggcctggtgtccctgatcctgctgctgctgggaagaaccagaggcaacagcgtgacccagatggaaggccctgtgactctgagcgaggaagccttcctgaccatcaactgcacctacacagccacaggctaccccagtctgttttggtacgtgcagtatcccggcgagggactgcagctgctgctcaaagccacaaaggccgacgacaagggcagcaacaagggctttgaggccacctaccggaaagagacaaccagcttccacctggaaaagggcagcgtccaggtgtccgattccgccgtgtatttttgtgccctgagatacggcaacaaccggctggcctttggcaagggcaatcaggtggtggtcatcccc

SEQ ID NO: 273 - DNA, 1389 bp, Artificial Sequence
  Synthetic seqeunce TCR1051 VB_CB_P2A_VA
atgggatctagactgctctgttgggtcctgctgtgtctgcttggagccggacctgtgaaagcaggcgtgacacagacacccagatacctgatcaagaccaggggccaacaagtgacactgagctgtagccctatctccggccacagaagcgtgtcctggtatcagcaaacccctggacagggcctccagttcctgttcgagtacttcagcgagacacagcggaacaagggcaacttccccggcagattttccggcagacagttcagcaactcccgcagcgagatgaacgtgtccacactggaactgggcgacagcgccctgtatctgtgcgcttctagtcctggcacaggcggcaataccgaggccttttttggccaaggcactcgcctgactgtggtggaggacctgaacaaagtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccaccatcctgtacgagatcctgctgggcaaggccacactgtacgccgtgctggtgtctgccctggtgctgatggccatggtcaagcggaaggacttcggttccggagccacgaacttctctctgttaaagcaagcaggagacgtggaagaaaaccccggtcccatgttgctgttgctggtgcccgccttccaagtgatctttaccctcggcggaacacgggcccagagtgtcacacagctggattctcaggtgcccgtgtttgaggaagcccctgtcgagctgagatgcaactacagcagctccgtgtccgtgtacctcttttggtatgttcagtaccccaaccagggcctgcagctcctcctgaagtatctgagcggcagcacactggtggaatccatcaatggcttcgaggccgagttcaacaagtcccagacctcattccatctgcggaagcccagcgtgcacatctctgataccgccgaatacttctgtgccgtgtccagcggcacctacaagtacatctttggcaccggcaccaggctgaaggtgctggcc

SEQ ID NO: 274 - DNA, 1398 bp, Artificial Sequence
  Synthetic seqeunce TCR1061 VB_CB_P2A_VA
atgggcacaagactgctgtgctgggtcgtgctgggctttctgggcacagatcatacaggcgccggtgtcagccagtctcctagatacaaggtggccaagcgcggacaggatgtggccctcagatgtgatcctatcagcggccacgtgtccctgttttggtatcagcaggccctcggacagggccccgagttcctgacctactttcagaatgaggctcagctggacaagagcggcctgcctagcgatagattcttcgccgaaagacccgagggcagcgtgtccacactgaagatccagagaacccagcaagaggacagcgccgtgtacctgtgtgccagcagttctccttctggcgccaacgtgctgacctttggcgctggctctagactgacagtgctggaggacctgaacaaagtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccaccatcctgtacgagatcctgctgggcaaggccacactgtacgccgtgctggtgtctgccctggtgctgatggccatggtcaagcggaaggacttcggttccggagccacgaacttctctctgttaaagcaagcaggagacgtggaagaaaaccccggtcccatggcctgtcctggatttctgtgggccctcgtgatcagcacctgtctggaattcagcatggcccagaccgtgacacagagccagcctgagatgtctgtgcaagaggccgagacagtgaccctgagctgcacctacgataccagcgagagcgactactacctgttctggtacaagcagcctcctagccggcagatgatcctggtcatcagacaagaggcctataagcagcagaacgccaccgagaacagattcagcgtgaacttccagaaggccgccaagagcttcagcctgaagatcagcgatagccagctgggcgacgccgccatgtacttttgtgccatcatgaccggcaccgccagcaagctgacatttggcaagggcaccctgctgaccgtgaatccc

SEQ ID NO: 275 - DNA, 1404 bp, Artificial Sequence
  Synthetic seqeunce TCR1072 VB_CB_P2A_VA
atgagccccatctttacctgcatcaccatcctgtgcctgctggccgctggatctcctggggaagaagtggcccagacacctaagcacctcgttagaggcgagggccagaaggccaagctgtattgcgcccctatcaagggccacagctatgttttttggtatcaacaggtcctgaagaacgagttcaagttcctgatcagcttccagaacgagaacgtgttcgacgagacaggcatgcccaaagagcggttctccgccaagtgcctgcctaacagcccttgcagcctggaaatccaggccaccaagctggaagattccgccgtgtatttctgcgccagcagcagcatgtctatcgccgctggaaataccggcgagctgttcttcggcgagggcagcagactgacagttctggacctgaagaacgtgttccccccagaggtggccgtgttcgagccttctgaggccgagatcagccacacccagaaagccaccctcgtgtgtctggccaccggcttttaccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgtctgccaccatcctgtacgagatcctgctgggaaaggccaccctgtacgccgtgctggtgtctgccctggtgctgatggccatggtcaagcggaaggacagcagaggcggttccggagccacgaacttctctctgttaaagcaagcaggagacgtggaagaaaaccccggtcccatgatttccctgagagtgctgctcgtgattctctggctccagctctcctgggtttggagccagcggaaagaggtcgagcaagaccctgggccttttaacgttccagagggcgctacagtggcttttaattgcacatactccaacagcgcctcacagagttttttctggtatcggcaggactgtagaaaagaaccgaaactgctcatgtccgtgtatagctccggcaatgaggatggccggtttaccgctcagctgaatcgggcctctcagtacatctccctgctgattcgggactccaagctgtccgatagcgcaacatacctgtgcgtggtcacaggcaccggcggcttcaagacaatcttcggagcaggcacccggctgtttgtgaaggct

SEQ ID NO: 276 - DNA, 25 bp, Artificial Sequence
  Synthetic seqeunce sgRNA Forward Oligo TRAC_sgRNA_pLenti_F1
caccggagaatcaaaatcggtgaat

SEQ ID NO: 277 - DNA, 25 bp, Artificial Sequence
  Synthetic seqeunce sgRNA Reverse Oligo TRAC_sgRNA_pLenti_R1
aaacattcaccgattttgattctcc

SEQ ID NO: 278 - DNA, 24 bp, Artificial Sequence
  Synthetic seqeunce PD1_sgRNA_F1
caccgcagttgtgtgacacggaag

SEQ ID NO: 279 - DNA, 24 bp, Artificial Sequence
  Synthetic seqeunce PD1_sgRNA_R1
aaaccttccgtgtcacacaactgc

SEQ ID NO: 280 - DNA, 25 bp, Artificial Sequence
  Synthetic seqeunce CTLA4_sgRNA_F1
caccggcaaaggtgagtgagacttt

SEQ ID NO: 281 - DNA, 25 bp, Artificial Sequence
  Synthetic seqeunce CTLA4_sgRNA_R1
aaacaaagtctcactcacctttgcc

SEQ ID NO: 282 - DNA, 25 bp, Artificial Sequence
  Synthetic seqeunce LAG3_sgRNA_F1
caccggtttctgcagccgctttggg

SEQ ID NO: 283 - DNA, 25 bp, Artificial Sequence
  Synthetic seqeunce LAG3_sgRNA_R1
aaaccccaaagcggctgcagaaacc

SEQ ID NO: 284 - protein, 9 aa, Artificial Sequence
  Synthetic seqeunce Merkel cell polyomavirus (MCPyV) T antigen
KLLEIAPNC

SEQ ID NO: 285 - protein, 9 aa, Artificial Sequence
  Synthetic seqeunce McPyV T antigen homolog
KLLEIAPNA

SEQ ID NO: 286 - protein, 9 aa, Artificial Sequence
  Synthetic seqeunce McPyV T antigen variant
KLLEISPNC

SEQ ID NO: 287 - protein, 9 aa, Artificial Sequence
  Synthetic seqeunce McPyV T antigen variant
KLLEITPNC

SEQ ID NO: 288 - protein, 9 aa, Artificial Sequence
  Synthetic seqeunce McPyV T antigen consensus sequence recognized by TCR1007
  VARIANT (1)...(9): Xaa = Any Amino Acid
KXLEIXXNX

SEQ ID NO: 289 - protein, 9 aa, Artificial Sequence
  Synthetic seqeunce McPyV T antigen consensus sequence recognized by TCR1072
  VARIANT (1)...(9): Xaa = Any Amino Acid
XLLEIAPNX

SEQ ID NO: 290 - protein, 214 aa, Artificial Sequence
  Synthetic seqeunce CD8 co-receptor alpha-chain
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 291 - protein, 189 aa, Artificial Sequence
  Synthetic seqeunce CD8 co-receptor beta-chain, isoform 1
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 292 - protein, 200 aa, Artificial Sequence
  Synthetic seqeunce CD8 co-receptor beta-chain, isoform 2
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 293 - protein, 225 aa, Artificial Sequence
  Synthetic seqeunce CD8 co-receptor beta-chain, isoform
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 294 - protein, 225 aa, Artificial Sequence; identical to SEQ ID NO: 293
  Synthetic seqeunce CD8 co-receptor beta-chain, isoform 4

SEQ ID NO: 295 - protein, 222 aa, Artificial Sequence
  Synthetic seqeunce CD8 co-receptor beta-chain, isoform 5
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 296 - DNA, 705 bp, Artificial Sequence
  Synthetic seqeunce CD8 co-receptor alpha-chain (M1), codon-optimized
atggctctgcctgtgacagctctgctgctgcctctggccctgctgctgcatgccgctagacccagccagttcagagtgtcccccctggacagaacctggaacctgggcgagacagtggaactgaagtgccaggtgctgctgagcaaccccaccagcggctgcagctggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtg

SEQ ID NO: 297 - DNA, 633 bp, Artificial Sequence
  Synthetic seqeunce CD8 co-receptor beta-chain (M1), codon-optimized
atgcggcccagactgtggctgctgctggctgctcagctgacagtgctgcacggcaactccgtgctgcagcagacccccgcctacatcaaggtgcagaccaacaagatggtcatgctgagctgcgaggccaagatcagcctgtccaacatgcggatctactggctgcggcagagacaggcccctagcagcgatagccaccacgagtttctggctctgtgggacagcgccaagggcaccattcacggcgaggaagtggaacaggaaaagatcgccgtgttccgggacgccagccggttcatcctgaacctgaccagcgtgaagcccgaggacagcggcatctatttctgcatgatcgtgggcagccccgagctgaccttcggcaagggaacacagctgagcgtggtggacttcctgcctaccaccgcccagcccaccaagaagtctaccctgaagaaaagagtgtgccggctgcccaggcccgagacacagaaaggccctctgtgcagccctatcaccctgggactgctggtggcaggggtgctggtgctgctggtgtctctgggagtggccatccacctgtgctgcaggcggagaagggccagactgcggttcatgaagcagttctacaagtga

SEQ ID NO: 298 - DNA, 1404 bp, Artificial Sequence
  Synthetic seqeunce CD8 co-receptor alpha-chain (M1)-P2A- CD8 co-receptor beta-chain (M1), codon-optimized
atggctctgcctgtgacagctctgctgctgcctctggccctgctgctgcatgccgctagacccagccagttcagagtgtcccccctggacagaacctggaacctgggcgagacagtggaactgaagtgccaggtgctgctgagcaaccccaccagcggctgcagctggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtgggttccggagccacgaacttctctctgttaaagcaagcaggagacgtggaagaaaaccccggtcccatgcggcccagactgtggctgctgctggctgctcagctgacagtgctgcacggcaactccgtgctgcagcagacccccgcctacatcaaggtgcagaccaacaagatggtcatgctgagctgcgaggccaagatcagcctgtccaacatgcggatctactggctgcggcagagacaggcccctagcagcgatagccaccacgagtttctggctctgtgggacagcgccaagggcaccattcacggcgaggaagtggaacaggaaaagatcgccgtgttccgggacgccagccggttcatcctgaacctgaccagcgtgaagcccgaggacagcggcatctatttctgcatgatcgtgggcagccccgagctgaccttcggcaagggaacacagctgagcgtggtggacttcctgcctaccaccgcccagcccaccaagaagtctaccctgaagaaaagagtgtgccggctgcccaggcccgagacacagaaaggccctctgtgcagccctatcaccctgggactgctggtggcaggggtgctggtgctgctggtgtctctgggagtggccatccacctgtgctgcaggcggagaagggccagactgcggttcatgaagcagttctacaagtga

SEQ ID NO: 299 - DNA, 1404 bp, Artificial Sequence
  Synthetic seqeunce CD8 co-receptor beta-chain (M1)-P2A- CD8 co-receptor alpha-chain (M1), codon-optimized
atgcggcccagactgtggctgctgctggctgctcagctgacagtgctgcacggcaactccgtgctgcagcagacccccgcctacatcaaggtgcagaccaacaagatggtcatgctgagctgcgaggccaagatcagcctgtccaacatgcggatctactggctgcggcagagacaggcccctagcagcgatagccaccacgagtttctggctctgtgggacagcgccaagggcaccattcacggcgaggaagtggaacaggaaaagatcgccgtgttccgggacgccagccggttcatcctgaacctgaccagcgtgaagcccgaggacagcggcatctatttctgcatgatcgtgggcagccccgagctgaccttcggcaagggaacacagctgagcgtggtggacttcctgcctaccaccgcccagcccaccaagaagtctaccctgaagaaaagagtgtgccggctgcccaggcccgagacacagaaaggccctctgtgcagccctatcaccctgggactgctggtggcaggggtgctggtgctgctggtgtctctgggagtggccatccacctgtgctgcaggcggagaagggccagactgcggttcatgaagcagttctacaagtgaggttccggagccacgaacttctctctgttaaagcaagcaggagacgtggaagaaaaccccggtcccatggctctgcctgtgacagctctgctgctgcctctggccctgctgctgcatgccgctagacccagccagttcagagtgtcccccctggacagaacctggaacctgggcgagacagtggaactgaagtgccaggtgctgctgagcaaccccaccagcggctgcagctggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtg

(19) United States
(12) Patent Application Publication
(10) Pub. No.: US 2021/0252057 A1
(43) Pub. Date: Aug. 19, 2021

(54) MERKEL CELL POLYOMAVIRUS T ANTIGEN-SPECIFIC TCRS AND USES THEREOF

(71) Applicants: FRED HUTCHINSON CANCER RESEARCH CENTER, Seattle, WA (US); UNIVERSITY OF WASHINGTON, Seattle, WA (US)

(72) Inventors: Aude CHAPUIS, Seattle, WA (US); Paul NGHIEM, Redmond, WA (US); Megan MCAFEE, Seattle, WA (US); Natalie MILLER, Seattle, WA (US); Kelly PAULSON, Shoreline, WA (US); David KOELLE, Seattle, WA (US); Thomas SCHMITT, Seattle, WA (US); Candice CHURCH, Seattle, WA (US)

(21) Appl. No.: 17/055,969

(22) PCT Filed: May 15, 2019
(86) PCT No.: PCT/US2019/032527

§ 371 Date: Nov. 16, 2020

Related U.S. Application Data
(60) Provisional application No. 62/672,232, filed on May 16, 2018.

(51) Int. Cl. A61K 35/17; A61K 38/20; A61K 38/21; C12N 5/0783

(52) U.S. Cl. CPC A61K 35/17; A61K 38/2013; C12N 2510/00; C12N 5/0638; C12N 2501/2302; A61K 38/215

(57) ABSTRACT

The present disclosure provides binding proteins and TCRs with high affinity and specificity against Merkel cell polyomavirus T antigen epitopes or peptides, T cells expressing such high affinity Merkel cell polyomavirus T antigen specific TCRs, nucleic acids encoding the same, and compositions for use in treating Merkel cell carcinoma.

### STATEMENT OF GOVERNMENT INTEREST

This invention was made with government support under CA176841 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_461WO_SEQUENCE_LISTING.txt. The text file is 136 KB, was created on May 10, 2019, and is being submitted electronically via EFS-Web.

### BACKGROUND

Adoptive transfer of tumor-specific T-cells is an appealing strategy to eliminate existing tumors and requires the establishment of a robust population of antigen-specific T cells in vivo to eliminate existing tumor and prevent recurrences (Stromnes et al., Immunol. Rev. 257:145, 2014). Although transfer of tumor-specific CD8⁺ cytotoxic T lymphocytes (CTLs) is safe and can mediate direct anti-tumor activity in select patients (Chapuis et al., Cancer Res. 72:LB-136, 2012; Chapuis et al., Sci. Transl. Med. 5:174ra127, 2013; Chapuis et al., Proc. Nat'l. Acad. Sci. U.S.A. 109:4592, 2012), the variability in the avidity of the CTLs isolated from each patient or donor limits the anti-tumor efficacy in clinical trials (Chapuis et al., 2013). Since TCR affinity is an important determinant of CTL avidity (Zoete et al., Frontiers Immunol. 4:268, 2013), strategies have been developed to redirect the antigen specificity of donor or patient T cells using high affinity TCRα/β genes isolated from a well-characterized T cell clone specific for a tumor-specific antigen (Stromnes et al., Immunol. Rev. 257:145, 2014; Robbins et al., J. Clin. Oncol. 29:917, 2011). Such high affinity self/tumor-reactive T cells are rare since T cells that express self/tumor-reactive TCRs are subject to central and peripheral tolerance (Stone and Kranz, Frontiers Immunol. 4:244, 2013), with relative TCR affinities varying widely between donors and patients. Therefore, many matched donors and patients must be screened to identify a sufficiently high-affinity antigen-specific T cell clone from which a TCRα/β gene therapy construct can be generated (see, e.g., Ho et al., J. Immunol. Methods 310:40, 2006).

Merkel cell carcinoma (MCC) is a rare, aggressive skin cancer with a reported incidence that has quadrupled since 1986 (Hodgson, J. Surg. Oncol. 89:1, 2005). There are currently over 2,000 new cases diagnosed each year in the United States (see Lemos and Nghiem, J. Invest. Dermatol. 127:2100, 2007), which is projected to almost double by the year 2025 (projected from Surveillance, Epidemiology, and End Results (SEER) Registry 18 data accessed January 2017, which is a program of the National Cancer Institute; see seer.cancer.gov). An increased risk of MCC has been linked with immunosuppression related to UV radiation, viral infections, organ transplantation, and chronic lymphocytic leukemia (Paulson et al., J. Invest. Dermatol. 129:1547, 2009; Goh et al., Oncotarget 7:3403, 2016; Feng et al., Science 319:1096, 2008). While MCC is more frequently observed in immunocompromised or elderly populations, more than 90% of patients with MCC do not appear to be observably immune compromised (Heath et al., J. Am. Acad. Dermatol. 58:375, 2008). Nonetheless, MCC is more lethal than melanoma with a reported 40% mortality rate (Heath et al., 2008), and MCC has a very poor prognosis once metastasized with a reported 5-year relative survival for patients having stage IV metastatic disease of only 18% (Lemos and Nghiem, 2007). To date, there is no established effective treatment for MCC patients. There are ongoing clinical trials using immune-modulation, such as immune checkpoint blocking antibodies (see Nghiem et al., N. Engl. J. Med. 374:2542, 2016; Kaufman et al., Lancet 17:1374, 2016) that result in only a 30% to 60% response rate, and targeted delivery of interleukin (IL)-2 (see www.immomec.eu)

Merkel cell polyomavirus (MCPyV) has been found to be associated with 80% of MCC cases (Garneski et al., Genome Biol. 9:228, 2008; Rodig et al., J. Clin. Invest. 122:4645, 2012), while the rest appear to be associated with UV-light exposure (Goh et al., 2016; Gonzalez-Vela et al., J. Invest. Dermatol. 137:197, 2017). Like other polyomaviruses, MCPyV contains two early genes that encode the large T antigen (LTA) and the small T antigen (STA), which are regarded as oncoproteins. LTA and STA share 78 amino acids at the amino-terminus and their expression appears to be necessary for the maintenance of MCC (Houben et al., J. Virol. 84:7064, 2010). The transforming activity of LTA appears to be related to a tumor-specific truncation mutation that eliminates the helicase domain (Shuda et al., Proc. Nat'l. Acad. Sci. USA 105:16272, 2008). Serologic studies have shown that anti-MCPyV antibodies are present in up to 88% of adults and more than 40% of children younger than 5 years (Pastrana et al., PLoS Pathogens 5:e1000578, 2009; Chen et al., J. Clin. Virol. 50:125, 2011), which indicates that MCPyV infection is common. But, antibodies against LTA and STA are largely restricted to patients with MCC and titers correlate with tumor burden (Paulson et al., Cancer Res. 70:8388, 2010). Many unique T cell epitopes in the MCPyV T proteins have been identified (Iyer et al., Clin. Cancer Res. 17:6671, 2011; Afanasiev et al., Clin. Cancer Res. 19:5351, 2013; Lyngaa et al., Clin. Cancer Res. 20:1768, 2014). Intratumoral CD8 T cell infiltration (also known as tumor infiltrating lymphocytes or TILs) has been has been correlated with increased survival of MCC patients, but only about a quarter of such patients have such immunity (Paulson et al., J. Clin. Oncol. 29:1539, 2011; Paulson et al., J. Invest. Dermatol. 133:642, 2013).

There is a need for highly antigen-specific TCR immunotherapies directed against Merkel cell carcinoma. Presently disclosed embodiments address these needs and provide other related advantages.

### BRIEF DESCRIPTION OF THE DRAWINGS

FIG. 1 shows expansion of (y-axis) and CD3 expression (x-axis) by untransduced CD8− Jurkat cells and CD8− T cells transduced with Merkel Cell Polyoma-Virus T antigen (MCPyV)-specific TCRs of the present disclosure, in response to peptide antigen:MHC multimers.

FIG. 2 provides a table summarizing the ability of the T cells shown in FIG. 1 to expand, express CD3, assemble heterologous TCR, and bind peptide:MHC the absence of CD8.

FIGS. 3A and 3B show recognition of MCPyV peptide antigen variants by a healthy donor-derived TCR of the present disclosure (“TCR1007”) and by comparator MCC patient-derived TCRs (“MCPyV TCR” in FIG. 3A; “389.6”, “389.7”, and “TCR1072” in FIG. 3B). (3A) CD8+ T cells transduced with the indicated MCPyV-specific TCRs were incubated overnight with antigen-presenting cells loaded with MCPyV variant peptides and analyzed for cytokine release and CD8 staining. (3B) Summary data showing the ability of T cells transduced with the indicated TCR to recognize variant peptides.

FIGS. 4A and 4B show that T cells transduced with a MCPyV-specific TCR of the present disclosure proliferate in response to endogenously presented MCPyV antigen. (4A) Flow cytometry showing expansion of CD8+ (top) and CD4+ (bottom) T cells transduced with the indicated MCPyV-specific TCRs. (4B) Percent of transduced CD8+ T cells that underwent at least one cell division when cultured with antigen-loaded APCs at the indicated effector:target (E:T) ratios.

FIGS. 5A-5C show that T cells transduced with a TCR of the present disclosure exhibit multiple functionalities in response to antigen. (A) Cytokine production. (B) Percent of TCR-transduced T cells that produced 0, 1, 2, or 3 cytokines when co-cultured with APCs loaded with the indicated concentrations of peptide. (C) Percentage of TCR-transduced T cells that produced IFN-γ in co-culture with APCs loaded with the indicated concentrations of peptide.

FIGS. 6A and 6B show that T cells transduced with a TCR of the present disclosure kill APCs loaded with peptide antigen (A) and MCPyV antigen-expressing SV40-transformed fibroblasts (B).

FIGS. 7A and 7B show that T cells transduced with TCRs of the present disclosure specifically kill MCPyV-expressing Merkel cells (WAGA cell line). (A) Specific lysis in the presence or absence of exogenously added IFN-γ. (B) HLA-A2 expression by target WAGA cells with (green line) or without (orange) exogenously added IFN-γ.

FIGS. 8A-8C show that TCRs of the present disclosure can engage CD4+ T cells. (A) Percent of CD4+ T cells transduced with the indicated TCR that underwent at least one division when co-cultured with antigen-loaded APCS at the indicated effector:target cell ratios. (B) Cytokine production by the CD4+ T cells. (C) Ability of TCR-transduced CD8+ (solid line) and TCR-transduced CD4+ (dashed line) T cells to specifically lyse target cells at the indicated effector:target ratios.

FIGS. 9A and 9B show that MCPyV-specific TCRs 1007 and 1072 require most residues of the peptide antigen for efficient recognition. Peptide residues (bottom; x-axis) were replaced by alanine as indicated and IFN-γ production by TCR-expressing CD8 T cells in response to the resultant variant peptide was measured. Residues at which alanine substitution resulted in a significant decrease in interferon production relative to the wild-type peptide are outlined in red.

FIG. 10 shows human peptide sequences with high sequence homology to the McPyV T-antigen consensus sequence required for efficient recognition by TCR1007. The peptide sequences shown in green were synthesized to determine whether TCR1007 posed a risk of cross-reactivity with these peptides in healthy human tissue.

FIG. 11 shows that TCR1007 does not produce cytokines in response to normal human peptide sequences with high homology to the TCR1007-recognized McPyV T-antigen consensus sequence.

FIG. 12 shows the results of on-going testing for potential alloreactivity of TCR1007 against HLA-A, -B, and -C alleles expressed on the indicated cell lines.

### DETAILED DESCRIPTION

The present disclosure generally provides T cell receptors (TCRs) having high affinity for Merkel Cell Polyomavirus (MCPyV) T antigen peptides associated with a major histocompatibility complex (WIC) (e.g., human leukocyte antigen, HLA) for use in, for example, adoptive immunotherapy to treat Merkel cell cancer (MCC). By way of background, Merkel cells are found in the epidermis and serve as touch cells by relaying touch-related information, such as texture and pressure, to the brain. While they are present in human skin at varying levels according to body site, they are at highest density on the fingertips and lips/face where touch sensation is most acute. In addition, they produce certain hormones and are sometimes referred to as neuroendocrine cells, although the reasons for which they produce certain hormones are unknown. Merkel cell carcinoma (MCC) is a rare, but highly aggressive, cutaneous neuroendocrine carcinoma, associated with the Merkel cell polyomavirus (MCPyV) in 80% of cases (Goh et al., 2016). The incidence of MCC is dramatically elevated in immunosuppressed patients (Ma and Brewer, Cancers 6:1328, 2014).

In virus-positive MCCs, the presumptive tumor antigens are non-self-proteins encoded by the viral genome (Paulson et al., 2010). An identified HLA-A*02:01 restricted MCPyV epitope is KLLEIAPNC (SEQ ID NO:284) (MCC/KLL) (Lyngaa et al., 2014), which has been associated with improved survival in patients. Therefore, MCPyV was targeted for immunotherapy due to its limited on target/off tissue toxicity therapeutic profile since it is a viral antigen only present in diseased tissue (Vandeven and Nghiem, Immunotherapy 8:907, 2016). One approach was to clonally expand the number of autologous MCPyV-specific T cells to promote a therapeutic effect in patients who control disease, but this was limited due to the insufficient numbers of MCPyV-specific T cells obtained (about 0.25% to 14% of the total dose needed, data not shown). Another drawback to this approach is that the avidity of the MCPyV-specific T cells obtained ranged over 3 orders of magnitude from one patient to another. In addition, this approach was limited by the fact that MCPyV-specific T cells could not be identified or grown in 86% of patients screened (n=69) (data not shown). Finally, even if cells could be clonally expanded, current procedures take more than about 2 months to generate cells of interest.

An advantage of the instant disclosure is to provide a high affinity binding protein or TCR specific for Merkel cell polyomavirus (MCPyV) T antigen (TA) epitopes present on TA protein, TA peptides and TA protein fragments, wherein a cell engineered to express such a binding protein or TCR is capable of binding to a TA-peptide:HLA complex and provide a therapeutic effect, optionally wherein the binding protein or TCR has high enough avidity to bind independent of CD8. In addition, such TCRs may be capable of more efficiently associating with a CD3 protein as compared to endogenous TCRs.

A method to quickly and simultaneously screen and rank T cell clonotypes (based on affinity for a Merkel cell polyomavirus T antigen) from a large cohort of HLA matched donors in a short time (about 6-8 weeks) comprised using limiting concentrations of a Merkel cell polyomavirus T antigen-specific pMHC multimers. The TCRβ repertoire was analyzed for frequency and then coupled with bioinformatics to accurately identify TCR α-chain and β-chain pairs.

The compositions and methods described herein will in certain embodiments have therapeutic utility for the treatment of diseases and conditions associated with a Merkel cell polyomavirus T antigen. Such diseases include various forms of hyperproliferative disorders, such as cancer. Exemplary uses include in vitro, ex vivo and in vivo stimulation of Merkel cell polyomavirus T antigen-specific T cell responses, such as by the use of modified T cells expressing an enhanced affinity TCR specific for a Merkel cell polyomavirus T antigen epitope or peptide.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising,” and refers to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

“Merkel cell carcinoma” or “MCC” or “neuroendocrine carcinoma of the skin,” as used herein, refers to hyperproliferative or uncontrolled growth of cells in the skin that share some characteristics with normal Merkel cells of the skin, which may be infected with a Merkel cell polyomavirus (MCPyV) or have a high somatic mutation burden (e.g., due to exposure to UV light) in one of more genes including RB1, TP53, chromatin modification pathway genes (e.g., ASXL1, MLL2, MLL3), JNK pathway genes (e.g., MAP3K1, TRAF7), and DNA-damage pathway (e.g., ATM, MSH2, BRCA1). The MCC arising from infection with MCPyV may also be referred to as “MCPyV-positive MCC” and MCC arising from a high somatic mutation burden may also be referred to as “MCPyV-negative MCC.”

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a γδ T cell, a stem cell memory T cell, a regulatory T cell, a natural killer cell, a natural killer T cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

“Major histocompatibility complex” (MHC) refers to glycoproteins that deliver peptide antigens to a cell surface. MHC class I molecules are heterodimers having a membrane spanning α chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

A “T cell” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4+ CD25+ (Foxp3+) regulatory T cells and Treg17 cells, as well as Tr1, Th3, CD8+CD28−, and Qa-1 restricted T cells.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like other immunoglobulins (e.g., antibodies), the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also like other immunoglobulins, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

“CD3” is known in the art as a multi-protein complex of six chains (see, Abbas and Lichtman, 2003; Janeway et al., p 172 and 178, 1999). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Without wishing to be bound by theory, it is believed the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCR chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, the term “CD8 co-receptor” or “CD8” means the cell surface glycoprotein CD8, which can form either an alpha-alpha homodimer or an alpha-beta heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are eight (8) different CD8 beta chain isoforms, four of which are expressed at the cell membrane and four of which are secreted (see UniProtKB identifier P10966), and a single CD8 alpha chain (see UniProtKB identifier P01732 and SEQ ID NO: 290)

“CD4” is an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to respectively bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target (e.g., Merkel cell polyomavirus T antigen, Merkel cell polyomavirus T antigen peptide:MHC complex). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv), receptor ectodomains, ligands (e.g., cytokines, chemokines), or synthetic polypeptides selected for their specific ability to bind to a biological molecule, a molecular complex or other target of interest.

As used herein, “specifically binds” or “specific for” refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

In certain embodiments, a polynucleotide encoding a TCR or binding protein of the present disclosure may be codon optimized to enhance expression in a particular host cell, such as T cells (Scholten et al., Clin. Immunol. 119:135, 2006). Codon optimization can be performed using known techniques and tools, e.g., using the GenScript® OptimumGene™ tool. Codon-optimized sequences include sequences that are at least partially codon-optimized (i.e., at least one codon is optimized for expression in the host cell) and those that are fully codon-optimized.

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

The term “Merkel cell polyomavirus T antigen-specific binding protein” or “MCPyV-T antigen-specific binding protein” refers to a protein or polypeptide that specifically binds to a Merkel cell polyomavirus T antigen epitope, peptide or T antigen fragment. In some embodiments, a protein or polypeptide specifically binds to a Merkel cell polyomavirus T antigen epitope or T antigen peptide thereof, such as a Merkel cell polyomavirus T antigen epitope peptide complexed with a MHC or an HLA molecule, e.g., on an immune cell surface, with at or at least about an avidity or affinity sufficient to elicit an immune response. In certain embodiments, a Merkel cell polyomavirus T antigen epitope-specific binding protein binds a Merkel cell polyomavirus T antigen-derived peptide:HLA complex (or MCPyV-T antigen-derived peptide:MHC complex) with a K_{d} of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary MCPyV-T antigen-specific binding protein provided herein, such as any of the MCPyV-T antigen-specific TCRs provided herein, for example, as measured by the same assay. In certain embodiments, a MCPyV-T antigen-specific binding protein comprises a MCPyV-T antigen-specific immunoglobulin superfamily binding protein or binding portion thereof.

Assays for assessing affinity or apparent affinity or relative affinity are known. In certain examples, apparent affinity for a TCR is measured by assessing binding to various concentrations of tetramers, for example, by flow cytometry using labeled tetramers. In some examples, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

The term “Merkel cell polyomavirus T antigen-specific binding domain” or “Merkel cell polyomavirus T antigen-specific binding fragment” refer to a domain or portion of a Merkel cell polyomavirus T antigen-specific binding protein responsible for the specific Merkel cell polyomavirus T antigen binding. A Merkel cell polyomavirus T antigen-specific binding domain alone (i.e., without any other portion of a Merkel cell polyomavirus T antigen-specific binding protein) can be soluble and can bind to a Merkel cell polyomavirus T antigen epitope or peptide with a K_{d} of less than about 10⁻⁸ M, less than about 10⁻⁹ M, less than about 10⁻¹⁰ M, less than about 10⁻¹¹ M, less than about 10¹² M, or less than about 10⁻¹³ M. Exemplary Merkel cell polyomavirus T antigen-specific binding domains include Merkel cell polyomavirus T antigen-specific scTCR (e.g., single chain αβTCR proteins such as Vα-L-Vβ, Vβ-L-Vα, Vα-Cα-L-Vα, or Vα-L-Vβ-Cβ, wherein Vα and Vβ are TCRα and β variable domains respectively, Cα and Cβ are TCRα and β constant domains, respectively, and L is a linker) and scFv fragments as described herein, which can be derived from an anti-Merkel cell polyomavirus T antigen TCR or antibody.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MEW gene that is relevant for antigen presentation) APC and T cells, are well established (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intrcellular pathogen) are generally from about 7 amino acids to about 11 amino acids in length and will associate with class I MEW molecules, whereas peptides processed in the vesicular system (e.g., bacterial, viral) will generally vary in length from about 10 amino acids to about 25 amino acids and associate with class II MEW molecules.

“Merkel cell polyomavirus T antigen” or “Merkel cell polyomavirus T antigen peptide” refer to a naturally or synthetically produced portion of a Merkel cell polyomavirus T antigen protein ranging in length from about 7 amino acids to about 15 amino acids, which can form a complex with a MHC (e.g., HLA) molecule and such a complex can bind with a TCR specific for a Merkel cell polyomavirus T antigen peptide:MHC (e.g., HLA) complex.

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, for instance, or about four to about 20 amino acids or about eight to about 15 amino acids or about 15 to about 25 amino acids. Exemplary linkers include Gycine-Serine (Gly-Ser) linkers, such as those provided in SEQ ID NOS:263 and 264.

“Junction amino acids” or “junction amino acid residues” refer to one or more (e.g., about 2-10) amino acid residues between two adjacent motifs, regions or domains of a polypeptide, such as between a binding domain and an adjacent constant domain or between a TCR chain and an adjacent self-cleaving peptide. Junction amino acids may result from the construct design of a fusion protein (e.g., amino acid residues resulting from the use of a restriction enzyme site during the construction of a nucleic acid molecule encoding a fusion protein).

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, TCRβ constant domain) of at least 85% (e.g., 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded. In certain embodiments, a sequence of two or more linked nucleic acid molecules is referred to as a polynucleotide.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

As used herein, the term “modified” or “genetically engineered” refers to a cell, microorganism, nucleic acid molecule, or vector that has been recombinantly created by human intervention—that is, modified by introduction of a heterologous nucleic acid molecule, or refers to a cell or microorganism that has been altered such that expression of an endogenous nucleic acid molecule or gene is controlled, deregulated or constitutive. Human-generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or two or three codons or amino acids, a deletion of one to about 5 codons or amino acids, or a combination thereof.

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2^{nd} Edition; Worth Publishers, Inc. NY, N.Y., pp. 71-77, 1975; Lewin, Genes IV, Oxford University Press, NY and Cell Press, Cambridge, Mass., p. 8, 1990).

The term “construct” refers to any polynucleotide that contains a recombinantly engineered nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The term “operably-linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or ‘transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by an exogenous nucleic acid molecule introduced into the host cell, even if the host cell encodes a homologous protein or activity.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding desired TCR specific for a Merkel cell polyomavirus T antigen peptide (e.g., TCRα and TCRβ). When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous or exogenous nucleic acid molecule may be homologous to a native host cell gene, and may optionally have an altered expression level, a different sequence, an altered activity, or any combination thereof.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻CD117⁺ phenotype or those found in the thymus (referred to as progenitor thymocytes).

As used herein, the term “host” refers to a cell (e.g., T cell) or microorganism targeted for genetic modification with a heterologous or exogenous nucleic acid molecule to produce a polypeptide of interest (e.g., high or enhanced affinity anti-Merkel cell polyomavirus T antigen TCR). A host cell may include any individual cell or cell culture which may receive a vector or the incorporation of nucleic acids or express proteins. The term also encompasses progeny of the host cell, whether genetically or phenotypically the same or different. It will be appreciated that a polynucleotide encoding a binding protein of this disclosure is “heterologous” with regard to progeny of a host cell of the present disclosure, as well as to the host cell.

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre-malignant cells, as well as non-neoplastic or non-malignant disorders

### Binding Proteins Specific for Merkel Cell Polyomavirus T Antigen Peptides

Ideal targets for immunotherapy are immunogenic proteins with high expression in malignant tissues and with limited-to-absent expression in normal tissues. As noted herein, Merkel cell polyomavirus (MCPyV) T antigen characteristics render it a good target for immunotherapy, including MCPyV having limited on target/off tissue toxicity due to the targeting of a viral antigen only present in diseased tissue (Vandeven and Nghiem, 2016).

Conservative substitutions of amino acids are well known and may occur naturally or may be introduced when the binding protein or TCR is genetically engineered. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, N Y, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al., supra).

A variety of criteria can be used to determine whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

Species (or variants) of a particular binding protein or high affinity T cell receptors (TCRs) specific for Merkel cell polyomavirus T antigen epitopes or peptides may have an amino acid sequence that has at least 85%, 90%, 95%, or 99% amino acid sequence identity to any of the exemplary amino acid sequences disclosed herein (e.g., SEQ ID NOS:65-82), provided that (a) at least three or four of the CDRs have no mutations, (b) the CDRs that do have mutations have only up to two amino acid substitutions, up to a contiguous five amino acid deletion, or a combination thereof, and (c) the binding protein retains its ability to bind to a Merkel cell polyomavirus T antigen peptide:HLA complex on a cell surface.

In any of the aforementioned embodiments, the present disclosure provides a T cell receptor (TCR), comprising an α-chain and a β-chain, wherein the TCR binds to Merkel cell polyomavirus T antigen peptide:HLA-A*201 complex on a cell surface.

In certain embodiments, a TCR according to the present disclosure or a binding domain thereof comprises a V_{α} domain and a V_{β} domain, wherein the V_{α} domain is derived from a variable (V) gene segment and a joining (J) gene segment according to Table 1, and wherein the V_{β} domain is derived from a V gene segment, a J gene segment, and a diversity (D) gene segment according to Table 1.

TABLE 1
V, D, and J Allele Usage by McPyV-Specific TCRs

| TCR Vα/Vβ | V | D | J |
|---|---|---|---|
| TCR1007 Vα | 23/DV6*01 | | J49*01 |
| TCR1007 Vβ | V07-08*01 | D02-01*01 | J02-07*01 |
| TCR1009 Vα | V19*01 | | J27*01 |
| TCR1009 Vβ | V06-01*01 | D01-01*01 | J01-05*01 |
| TCR1012 Vα | V3*01 | | J10*01 |
| TCR1012 Vβ | V07-08*01 | D01-01*01 | J02-07*01 |
| TCR1016 Vα | V14/DV4*01 | | J13*01 |
| TCR1016 Vβ | V13-01*01 | D01-01*01 | J02-01*01 |
| TCR1021 Vα | V8-2*01 | | J3*01 |
| TCR1021 Vβ | V06-06*01 | D02-01*02 | J02-01*01 |
| TCR1027 Vα | V12-2*01 | | J49*01 |
| TCR1027 Vβ | V05-05*01 | D02-01*01 | J02-07*01 |
| TCR1034 Vα | V25*01 | | J20*01 |
| TCR1034 Vβ | V12-03*01 | D01-01*01 | J02-01*01 |
| TCR1042 Vα | V9-2*01 | | J7*01 |
| TCR1042 Vβ | V03-01*01 | D02-01*01 | J02-01*01 |
| TCR1051 Vα | V8-6*01 | | J40*01 |
| TCR1051 Vβ | V05-01*01 | D01-01*01 | J01-01*01 |
| TCR1061 Vα | V38-2/DV8*01 | | J53*01 |
| TCR1061 Vβ | V07-08*01 | D02-01*01 | J02-06*01 |
| TCR1072 Vα | V12-1 | | J9 |
| TCR1072 Vβ | V6 | D02-01*01 | J2-2 |

In certain embodiments, a TCR according to the present disclosure or a binding domain thereof comprises a Vα domain and a VP domain, wherein the Vα domain is derived from any variable (V) gene segment and any joining (J) gene segment set forth in Table 1, and wherein the Vβ domain is derived from any V gene segment, any J gene segment, and any diversity (D) gene segment set forth in Table 1.

In certain embodiments, this disclosure provides a method for treating Merkel cell carcinoma by administering to a subject having, or at risk of having, Merkel cell carcinoma a therapeutically effective amount of a modified immune cell (e.g., a host T cell) comprising a heterologous nucleic acid molecule encoding a binding protein specific for a Merkel cell polyomavirus T antigen, such as a Merkel cell polyomavirus T antigen, a Merkel cell polyomavirus T antigen peptide, or a Merkel cell polyomavirus T antigen peptide:HLA complex.

In any of the herein disclosed exemplary embodiments, an encoded binding protein of this disclosure comprises: (a) a T cell receptor (TCR) α chain variable (Vα) domain having a CDR3 amino acid sequence according to any one of SEQ ID NOS.:7, 13, 19, 25, 31, 37, 43, 49, and 55, and a TCR β chain variable (Vβ) domain; (b) a Vβ domain having a CDR3 amino acid sequence according to any one of SEQ ID NOS.:10, 16, 22, 28, 34, 40, 46, 52, and 58, and a Vα domain; or (c) a Vα domain having a CDR3 amino acid sequence according to any one of SEQ ID NOS:7, 13, 19, 25, 31, 37, 43, 49, and 55, and a Vβ domain having a CDR3 amino acid sequence according to any one of SEQ ID NOs:10, 16, 22, 28, 34, 40, 46, 52, and 58; and wherein the binding protein is capable of specifically binding to a Merkel cell polyomavirus T antigen peptide:HLA complex on a cell surface.

In certain embodiments, an encoded binding protein comprises (i) a Vα domain having a CDR3 amino acid sequence according to SEQ ID NO:7 and a Vβ domain having a CDR3 amino acid sequence according to SEQ ID NO:10; (ii) a Vα domain having a CDR3 amino acid sequence according to SEQ ID NO:13 and a Vβ domain having a CDR3 amino acid sequence according to SEQ ID NO:16; (iii) a Vα domain having a CDR3 amino acid sequence according to SEQ ID NO:19 and a Vβ domain having a CDR3 amino acid sequence according to SEQ ID NO:22; (iv) a Vα domain having a CDR3 amino acid sequence according to SEQ ID NO:25 and a Vβ domain having a CDR3 amino acid sequence according to SEQ ID NO:28; (v) a Vα domain having a CDR3 amino acid sequence according to SEQ ID NO:31 and a Vβ domain having a CDR3 amino acid sequence according to SEQ ID NO:34; (vi) a Vα domain having a CDR3 amino acid sequence according to SEQ ID NO:37 and a Vβ domain having a CDR3 amino acid sequence according to SEQ ID NO:40; (vii) a Vα domain having a CDR3 amino acid sequence according to SEQ ID NO:43 and a Vβ domain having a CDR3 amino acid sequence according to SEQ ID NO:46; (viii) a Vα domain having a CDR3 amino acid sequence according to SEQ ID NO:49 and a Vβ domain having a CDR3 amino acid sequence according to SEQ ID NO:52; or (ix) a Vα domain having a CDR3 amino acid sequence according to SEQ ID NO:55 and a Vβ domain having a CDR3 amino acid sequence according to SEQ ID NO:58.

In any of the herein disclosed embodiments, an encoded binding protein comprises a Vα domain that is at least about 90% (i.e., at least about 90%, 91%, 92%, 93%, 94%, 95%, 96%, 97%, 98%, 99%, or 100%) identical to an amino acid sequence of SEQ ID NO: 65, 67, 69, 71, 73, 75, 77, 79, or 81, and comprises a Vβ domain that is at least about 90% (i.e., at least about 90%, 91%, 92%, 93%, 94%, 95%, 96%, 97%, 98%, 99%, or 100%) identical to an amino acid sequence of SEQ ID NO: 66, 68, 70, 72, 74, 76, 78, 80, or 82, provided that (a) at least three or four of the CDRs have no change in sequence, wherein the CDRs that do have sequence changes have only up to two amino acid substitutions, up to a contiguous five amino acid deletion, or a combination thereof, and (b) the binding protein remains capable of specifically binding to a Merkel cell polyomavirus T antigen peptide:HLA cell surface complex.

In any of the herein disclosed embodiments, the encoded binding protein is capable of specifically binding a KLLEIAPNC (SEQ ID NO:284):human leukocyte antigen (HLA) complex or a KLLEIAPNA (SEQ ID NO:285):human leukocyte antigen (HLA) complex.

In any of the herein disclosed embodiments, an encoded binding protein of this disclosure comprises (a) an encoded Vα domain comprising (i) a CDR1 amino acid sequence according to any one of SEQ ID NOS:9, 15, 21, 27, 33, 39, 45, 51, and 57, and/or (ii) a CDR2 amino acid sequence according to any one of SEQ ID NOS:8, 14, 20, 26, 32, 38, 44, 50, and 56; and/or (b) an encoded Vβ domain comprising (i) a CDR1 amino acid sequence according to any one of SEQ ID NOS:12, 18, 24, 30, 36, 42, 48, 54, and 60, and/or (ii) a CDR2 amino acid sequence according to any one of SEQ ID NOS:11, 17, 23, 29, 35, 41, 47, 53, and 59.

In certain embodiments, an encoded binding protein of this disclosure comprises a Vα CDR1, a Vα CDR2, a Vβ CDR1, and a Vβ CDR2 according to: (i) SEQ ID NOs:9, 8, 12, and 11, respectively; (ii) SEQ ID NOs:15, 14, 18, and 17, respectively; (iii) SEQ ID NOs:21, 20, 24, and 23, respectively; (iv) SEQ ID NOs:27, 26, 30, and 29, respectively; (v) SEQ ID NOs:33, 32, 36, and 35, respectively; (vi) SEQ ID NOs:39, 38, 42, and 41, respectively; (vii) SEQ ID NOs:45, 44, 48, and 47, respectively; (vii) SEQ ID NOs:51, 50, 54, and 53, respectively; or (ix) SEQ ID NOs:57, 56, 60, and 59, respectively.

In particular embodiments, an encoded binding protein of this disclosure comprises: (a) Vα CDR1, CDR2, and CDR3 amino acid sequences according to SEQ ID NOS:9, 8, and 7, respectively, and Vβ CDR1, CDR2, and CDR3 amino acid sequences according to SEQ ID NOS:12, 11, and 10, respectively; (b) Vα CDR1, CDR2, and CDR3 amino acid sequences according to SEQ ID NOS:15, 14, and 13, respectively, and Vβ CDR1, CDR2, and CDR3 amino acid sequences according to SEQ ID NOS:18, 17, and 16, respectively; (c) Vα CDR1, CDR2, and CDR3 amino acid sequences according to SEQ ID NOS:21, 20, and 19, respectively, and Vβ CDR1, CDR2, and CDR3 amino acid sequences according to SEQ ID NOS:24, 23, and 22, respectively; (d) Vα CDR1, CDR2, and CDR3 amino acid sequences according to SEQ ID NOS:27, 26, and 25, respectively, and Vβ CDR1, CDR2, and CDR3 amino acid sequences according to SEQ ID NOS:30, 29, and 28, respectively; (e) Vα CDR1, CDR2, and CDR3 amino acid sequences according to SEQ ID NOS:33, 32, and 31, respectively, and Vβ CDR1, CDR2, and CDR3 amino acid sequences according to SEQ ID NOS:36, 35, and 34, respectively; (f) Vα CDR1, CDR2, and CDR3 amino acid sequences according to SEQ ID NOS:39, 38, and 37, respectively, and Vβ CDR1, CDR2, and CDR3 amino acid sequences according to SEQ ID NOS:42, 41, and 40, respectively; (g) Vα CDR1, CDR2, and CDR3 amino acid sequences according to SEQ ID NOS:45, 44, and 43, respectively, and Vβ CDR1, CDR2, and CDR3 amino acid sequences according to SEQ ID NOS:48, 47, and 46, respectively; (h) Vα CDR1, CDR2, and CDR3 amino acid sequences according to SEQ ID NOS:51, 50, and 49, respectively, and Vβ CDR1, CDR2, and CDR3 amino acid sequences according to SEQ ID NOS:54, 53, and 52, respectively; or (i) Vα CDR1, CDR2, and CDR3 amino acid sequences according to SEQ ID NOS:57, 56, and 55, respectively, and Vβ CDR1, CDR2, and CDR3 amino acid sequences according to SEQ ID NOS:60, 59, and 58, respectively.

In any of the embodiments disclosed herein, an encoded binding protein specifically binds to a KLLEIAPNC (SEQ ID NO:284):HLA-A*201 complex.

In any of the embodiments disclosed herein, an encoded Vα domain comprises or consists of an amino acid sequence having at least about 85% identity (i.e., at least about 85%, 90%, 91%, 92%, 93%, 94%, 95%, 96%, 97%, 98%, 99%, or 100%) to SEQ ID NO.:65, 67, 69, 71, 73, 75, 77, 79, or 81. In any of the herein disclosed embodiments, an encoded Vβ domain comprises or consists of an amino acid sequence having at least about 85% identity (i.e., at least about 85%, 90%, 91%, 92%, 93%, 94%, 95%, 96%, 97%, 98%, 99%, or 100%) to SEQ ID NO.: 66, 68, 70, 72, 74, 76, 78, 80, or 82.

In particular embodiments, (a) the encoded Vα domain comprises or consists of the amino acid sequence according to SEQ ID NO.:65 and the encoded Vβ domain comprises or consists of the amino acid sequence according to SEQ ID NO.:66; (b) the encoded Vα domain comprises or consists of the amino acid sequence according to SEQ ID NO.:67 and the encoded Vβ domain comprises or consists of the amino acid sequence according to SEQ ID NO.:68; (c) the encoded Vα domain comprises or consists of the amino acid sequence according to SEQ ID NO.:69 and the encoded Vβ domain comprises or consists of the amino acid sequence according to SEQ ID NO.:70; (d) the encoded Vα domain comprises or consists of the amino acid sequence according to SEQ ID NO.:71 and the encoded Vβ domain comprises or consists of the amino acid sequence according to SEQ ID NO.:72; (e) the encoded Vα domain comprises or consists of the amino acid sequence according to SEQ ID NO.:73 and the encoded Vβ domain comprises or consists of the amino acid sequence according to SEQ ID NO.:74; (f) the encoded Vα domain comprises or consists of the amino acid sequence according to SEQ ID NO.:75 and the encoded Vβ domain comprises or consists of the amino acid sequence according to SEQ ID NO.:76; (g) the encoded Vα domain comprises or consists of the amino acid sequence according to SEQ ID NO.:77 and the encoded Vβ domain comprises or consists of the amino acid sequence according to SEQ ID NO.:78; (h) the encoded Vα domain comprises or consists of the amino acid sequence according to SEQ ID NO.:79 and the encoded Vβ domain comprises or consists of the amino acid sequence according to SEQ ID NO.:80; or (i) the encoded Vα domain comprises or consists of the amino acid sequence according to SEQ ID NO.:81 and the encoded Vβ domain comprises or consists of the amino acid sequence according to SEQ ID NO.:82.

In yet further embodiments, a modified immune cell according to the present disclosure further comprises a heterologous polynucleotide encoding a TCR α chain constant domain (Cα), a heterologous polynucleotide encoding a TCR β chain constant domain (Cβ), or both. In certain embodiments, the encoded Cα domain comprises an amino acid sequence with at least about 90% sequence identity to an amino acid sequence according to SEQ ID NO.:85. In certain embodiments, the encoded Cβ domain comprises an amino acid sequence with at least about 90% sequence identity to the amino acid sequence according to SEQ ID NO.:86 or 87.

In some embodiments, an encoded binding protein comprises: a Vα domain comprising or consisting of SEQ ID NO.:65, a Vβ domain comprising or consisting of SEQ ID NO.:66, a Cα domain comprising or consisting of SEQ ID NO.:85, and a Cβ domain comprising or consisting of SEQ ID NO.:86; a Vα domain comprising or consisting of SEQ ID NO.:67, a Vβ domain comprising or consisting of SEQ ID NO.:68, a Cα domain comprising or consisting of SEQ ID NO.:85, and a Cβ domain comprising or consisting of SEQ ID NO.:87; a Vα domain comprising or consisting of SEQ ID NO.:69, a Vβ domain comprising or consisting of SEQ ID NO.:70, a Cα domain comprising or consisting of SEQ ID NO.:85, and a Cβ domain comprising or consisting of SEQ ID NO.:87; a Vα domain comprising or consisting of SEQ ID NO.:71, a Vβ domain comprising or consisting of SEQ ID NO.:72, a Cα domain comprising or consisting of SEQ ID NO.:85, and a Cβ domain comprising or consisting of SEQ ID NO.:87; a Vα domain comprising or consisting of SEQ ID NO.:73, a Vβ domain comprising or consisting of SEQ ID NO.:74, a Cα domain comprising or consisting of SEQ ID NO.:85, and a Cβ domain comprising or consisting of SEQ ID NO.:87; a Vα domain comprising or consisting of SEQ ID NO.:75, a Vβ domain comprising or consisting of SEQ ID NO.:76, a Cα domain comprising or consisting of SEQ ID NO.:85, and a Cβ domain comprising or consisting of SEQ ID NO.:86; a Vα domain comprising or consisting of SEQ ID NO.:77, a Vβ domain comprising or consisting of SEQ ID NO.:78, a Cα domain comprising or consisting of SEQ ID NO.:85, and a Cβ domain comprising or consisting of SEQ ID NO.:87; a Vα domain comprising or consisting of SEQ ID NO.:79, a Vβ domain comprising or consisting of SEQ ID NO.:80, a Cα domain comprising or consisting of SEQ ID NO.:85, and a Cβ domain comprising or consisting of SEQ ID NO.:86; or a Vα domain comprising or consisting of SEQ ID NO.:81, a Vβ domain comprising or consisting of SEQ ID NO.:82, a Cα domain comprising or consisting of SEQ ID NO.:85, and a Cβ domain comprising or consisting of SEQ ID NO.:87.

In certain embodiments, any of the aforementioned Merkel cell polyomavirus T antigen specific binding proteins are each a T cell receptor (TCR), a chimeric antigen receptor or an antigen-binding fragment of a TCR, any of which can be chimeric, humanized or human. In further embodiments, an antigen-binding fragment of a TCR comprises a single chain TCR (scTCR) or is contained in a chimeric antigen receptor (CAR). In certain embodiments, a Merkel cell polyomavirus T antigen specific binding protein is a TCR, optionally a scTCR. Methods for producing engineered TCRs are described in, for example, Bowerman et al. (Mol. Immunol. 46:3000, 2009), the techniques of which are herein incorporated by reference. In certain embodiments, a Merkel cell polyomavirus T antigen-specific binding domain comprises a CAR comprising a Merkel cell polyomavirus T antigen-specific TCR binding domain (see, e.g., Walseng et al., Scientific Reports 7:10713, 2017; the TCR CAR constructs of which are hereby incorporated by reference in their entirety). Methods for making CARs are also described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., Clin. Cancer Res. 13:5426, 2007, the techniques of which are herein incorporated by reference.

It will be understood that any of the herein disclosed encoded binding proteins or co-receptor proteins can comprise a signal peptide” (also known as a leader sequence, leader peptide, or transit peptide), or can have a signal peptide removed or altered as compared to a signal peptide present in a disclosed sequence. Signal peptides can target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In any of the herein disclosed embodiments, an encoded binding protein may comprise a TCR variable domain (e.g., α, β) or TCR chain sequence (e.g., α, β) amino acid sequence as disclosed herein, but with a signal peptide portion removed or altered. In general, signal peptides range from about 18 or 19 to about 20, 21, 22, 23, or 24 amino acids at the amino-terminal end of an encoded polypeptide, and will be recognized or readily deduced from a sequence by those having ordinary skill in the art. Thus, any binding protein or co-receptor protein or fragment or portion thereof of the present disclosure can comprise a corresponding amino acid sequence as disclosed herein in which a signal peptide is present, altered, or absent. In certain embodiments, a binding protein sequence such as TCR variable domain (e.g., α, β) or TCR chain sequence (e.g., α, β) amino acid sequence is comprised in a pre-protein. In certain embodiments, a binding protein of the present disclosure is a mature protein.

In any of the aforementioned embodiments, the present disclosure provides a Merkel cell polyomavirus T antigen-specific binding protein that comprises a V_{α} domain having an amino acid sequence as disclosed herein, a TCR α-chain constant domain having an amino acid sequence as disclosed herein, a V_{β} domain having an amino acid sequence as disclosed herein, or a TCR β-chain constant domain having an amino acid sequence as disclosed herein, or any combination thereof. In certain embodiments, there is provided a composition comprising a Merkel cell polyomavirus T antigen peptide-specific binding protein or high affinity TCR according to any one of the aforementioned embodiments and a pharmaceutically acceptable carrier, diluent, or excipient.

Methods useful for isolating and purifying genetically engineered soluble TCR may include, by way of example, obtaining supernatants from suitable host cell/vector systems that secrete the genetically engineered soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/genetically engineered soluble TCR described herein include batch cell culture, which is monitored and controlled to maintain appropriate culture conditions. Purification of the soluble TCR may be performed according to methods described herein and known in the art and that comport with laws and guidelines of domestic and foreign regulatory agencies.

Merkel cell polyomavirus T antigen-specific binding proteins or domains, as described herein, may be functionally characterized according to methodologies used for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

“MHC-peptide tetramer staining” refers to an assay used to detect antigen-specific T cells, which features a tetramer of MHC molecules, each comprising an identical peptide having an amino acid sequence that is cognate (e.g., identical or related to) at least one antigen (e.g., Merkel cell polyomavirus T antigen), wherein the complex is capable of binding T cell receptors specific for the cognate antigen. Each of the MHC molecules may be tagged with a biotin molecule. Biotinylated MHC/peptides are tetramerized by the addition of streptavidin, which can be fluorescently labeled. The tetramer may be detected by flow cytometry via the fluorescent label. In certain embodiments, an MHC-peptide tetramer assay is used to detect or select enhanced affinity TCRs of the instant disclosure.

Levels of cytokines may be determined according to methods described herein, including the use of ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

### Polynucleotides Encoding Binding Proteins Specific for Merkel Cell Polyomavirus T Antigen

In certain embodiments, nucleic acid molecules encoding an immunoglobulin superfamily binding protein or high affinity TCR specific for Merkel cell polyomavirus T antigen are used to transfect/transduce a host cell (e.g., T cells) for use in adoptive transfer therapy. Advances in TCR sequencing have been described (e.g., Robins et al., Blood 114:4099, 2009; Robins et al., Sci. Translat. Med. 2:47ra64, 2010; Robins et al., (September 10) J. Imm. Meth. Epub ahead of print, 2011; Warren et al., Genome Res. 21:790, 2011) and may be employed in the course of practicing the embodiments according to the present disclosure. Similarly, methods for transfecting/transducing T cells with desired nucleic acids have been described (e.g., U.S. Patent Application Pub. No. US 2004/0087025) as have adoptive transfer procedures using T cells of desired antigen-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 09:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to high affinity TCRs specific for Merkel cell polyomavirus T antigen peptides complexed with an HLA receptor.

Construction of an expression vector that is used for genetically engineering a binding protein or high affinity engineered TCR specific for a Merkel cell polyomavirus T antigen peptide of interest can be accomplished by using, for example, restriction endonuclease digestion, ligation, transformation, plasmid purification, and DNA sequencing as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). To obtain efficient transcription and translation, a polynucleotide in each genetically engineered expression construct includes at least one appropriate expression control sequence (also called a regulatory sequence), such as a promoter operably (i.e., operatively) linked to the nucleotide sequence encoding the binding protein. In certain embodiments, a nucleic acid encoding a binding protein of this disclosure will further include a polynucleotide encoding a leader sequence.

Certain embodiments relate to nucleic acids that encode the polypeptides contemplated herein, for instance, binding proteins or high affinity TCRs specific for Merkel cell polyomavirus T antigen. As one of skill in the art will recognize, a nucleic acid may refer to a single- or a double-stranded DNA, cDNA or RNA in any form, and may include a positive and a negative strand of the nucleic acid which complement each other, including anti-sense DNA, cDNA and RNA. Also included are siRNA, microRNA, RNA-DNA hybrids, ribozymes, and other various naturally occurring or synthetic forms of DNA or RNA.

In any of the embodiments disclosed herein, a polynucleotide (e.g., a polynucleotide encoding a binding protein of the instant disclosure or a portion thereof (e.g., a CDR, a Vα domain, a Vβ domain, a TCRα chain, a TCR β chain, and the like), encoding a CD8 co-receptor or an extracellular portion thereof, or encoding both a binding protein or a portion thereof and a CD8 co-receptor or an extracellular portion thereof) is codon-optimized for efficient expression in a target host cell. In certain embodiments, any or all polynucleotides of the present disclosure are codon-optimized for expression in a T cell.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

In certain embodiments, the instant disclosure provides an isolated polynucleotide encoding a binding protein having a TCR Vα domain and a TCR Vβ domain, wherein the encoded binding protein is capable of specifically binding to a Merkel cell polyomavirus T antigen peptide:HLA complex on a cell surface, the isolated polynucleotide comprising: (a) a Vα CDR3-encoding polynucleotide according to SEQ ID NO:154, 160, 166, 172, 178, 184, 190, 196, or 202, and a Vβ-encoding polynucleotide; (b) a Vβ CDR3-encoding polynucleotide according to SEQ ID NO:157, 163, 169, 175, 181, 187, 193, 199, or 205, and a Vα-encoding polynucleotide; or (c) a Vα CDR3-encoding polynucleotide according to SEQ ID NO: 154, 160, 166, 172, 178, 184, 190, 196, or 202, and a Vβ CDR3-encoding polynucleotide according to SEQ ID NO: SEQ ID NO:157, 163, 169, 175, 181, 187, 193, 199, or 205.

In certain embodiments, a Vβ-encoding polynucleotide is derived from any combination of V, D, and J alleles according to Table 1. In certain embodiments, a Vα-encoding polynucleotide is derived from any combination of V and J alleles according to Table 1.

In further embodiments, an isolated polynucleotide encoding a binding protein according to the present disclosure comprises: (a) a Vα CDR3-encoding polynucleotide according to SEQ ID NO:154 and a Vβ CDR3-encoding polynucleotide according to SEQ ID NO:157; (b) a Vα CDR3-encoding polynucleotide according to SEQ ID NO:160 and a Vβ CDR3-encoding polynucleotide according to SEQ ID NO:163; (c) a Vα CDR3-encoding polynucleotide according to SEQ ID NO:166 and a Vβ CDR3-encoding polynucleotide according to SEQ ID NO:169; (d) a Vα CDR3-encoding polynucleotide according to SEQ ID NO:172 and a Vβ CDR3-encoding polynucleotide according to SEQ ID NO:175; (e) a Vα CDR3-encoding polynucleotide according to SEQ ID NO:178 and a Vβ CDR3-encoding polynucleotide according to SEQ ID NO:181; (f) a Vα CDR3-encoding polynucleotide according to SEQ ID NO:184 and a Vβ CDR3-encoding polynucleotide according to SEQ ID NO:187; (g) a Vα CDR3-encoding polynucleotide according to SEQ ID NO:190 and a Vβ CDR3-encoding polynucleotide according to SEQ ID NO:193; (h) a Vα CDR3-encoding polynucleotide according to SEQ ID NO:196 and a Vβ CDR3-encoding polynucleotide according to SEQ ID NO:199; or (i) a Vα CDR3-encoding polynucleotide according to SEQ ID NO:202 and a Vβ CDR3-encoding polynucleotide according to SEQ ID NO:205.

In any of the herein disclosed embodiments, an isolated polynucleotide encoding a binding protein according to the present disclosure comprises: (a) a Vα CDR1-encoding polynucleotide according to SEQ ID NO:156, 162, 168, 174, 180, 186, 192, 198, or 204; (b) a Vα CDR2-encoding polynucleotide according to SEQ ID NO:155, 161, 167, 173, 179, 185, 191, 197, or 203; (c) a Vβ CDR1-encoding polynucleotide according to SEQ ID NO:159, 165, 171, 177, 183, 189, 195, 201, or 207; and/or (d) a Vβ CDR2-encoding polynucleotide according to SEQ ID NO:158, 164, 170, 176, 184, 188, 194, 200, or 206.

In particular embodiments, an isolated polynucleotide comprises: (a) a Vα CDR1-encoding polynucleotide according to SEQ ID NO:156, a Vα CDR2-encoding polynucleotide according to SEQ ID NO:155, a Vα CDR3-encoding polynucleotide according to SEQ ID NO:154, a Vβ CDR1-encoding polynucleotide according to SEQ ID NO:159, a Vβ CDR2-encoding polynucleotide according to SEQ ID NO:158, and Vβ CDR3-encoding polynucleotide according to SEQ ID NO:157; (b) a Vα CDR1-encoding polynucleotide according to SEQ ID NO:162, a Vα CDR2-encoding polynucleotide according to SEQ ID NO:161, a Vα CDR3-encoding polynucleotide according to SEQ ID NO:160, a Vβ CDR1-encoding polynucleotide according to SEQ ID NO:165, a Vβ CDR2-encoding polynucleotide according to SEQ ID NO:164, and Vβ CDR3-encoding polynucleotide according to SEQ ID NO:163; (c) a Vα CDR1-encoding polynucleotide according to SEQ ID NO:168, a Vα CDR2-encoding polynucleotide according to SEQ ID NO:167, a Vα CDR3-encoding polynucleotide according to SEQ ID NO:166, a Vβ CDR1-encoding polynucleotide according to SEQ ID NO:171, a Vβ CDR2-encoding polynucleotide according to SEQ ID NO:170, and Vβ CDR3-encoding polynucleotide according to SEQ ID NO:169; (d) a Vα CDR1-encoding polynucleotide according to SEQ ID NO:174, a Vα CDR2-encoding polynucleotide according to SEQ ID NO:173, a Vα CDR3-encoding polynucleotide according to SEQ ID NO:172, a Vβ CDR1-encoding polynucleotide according to SEQ ID NO:177, a Vβ CDR2-encoding polynucleotide according to SEQ ID NO:176, and Vβ CDR3-encoding polynucleotide according to SEQ ID NO:175; (e) a Vα CDR1-encoding polynucleotide according to SEQ ID NO:180, a Vα CDR2-encoding polynucleotide according to SEQ ID NO:179, a Vα CDR3-encoding polynucleotide according to SEQ ID NO:178, a Vβ CDR1-encoding polynucleotide according to SEQ ID NO:183, a Vβ CDR2-encoding polynucleotide according to SEQ ID NO:182, and Vβ CDR3-encoding polynucleotide according to SEQ ID NO:181; (f) a Vα CDR1-encoding polynucleotide according to SEQ ID NO:186, a Vα CDR2-encoding polynucleotide according to SEQ ID NO:185, a Vα CDR3-encoding polynucleotide according to SEQ ID NO:184, a Vβ CDR1-encoding polynucleotide according to SEQ ID NO:189, a Vβ CDR2-encoding polynucleotide according to SEQ ID NO:188, and Vβ CDR3-encoding polynucleotide according to SEQ ID NO:187; (g) a Vα CDR1-encoding polynucleotide according to SEQ ID NO:192, a Vα CDR2-encoding polynucleotide according to SEQ ID NO:191, a Vα CDR3-encoding polynucleotide according to SEQ ID NO:190, a Vβ CDR1-encoding polynucleotide according to SEQ ID NO:195, a Vβ CDR2-encoding polynucleotide according to SEQ ID NO:194, and Vβ CDR3-encoding polynucleotide according to SEQ ID NO:193; (h) a Vα CDR1-encoding polynucleotide according to SEQ ID NO:198, a Vα CDR2-encoding polynucleotide according to SEQ ID NO:197, a Vα CDR3-encoding polynucleotide according to SEQ ID NO:196, a Vβ CDR1-encoding polynucleotide according to SEQ ID NO:201, a Vβ CDR2-encoding polynucleotide according to SEQ ID NO:200, and Vβ CDR3-encoding polynucleotide according to SEQ ID NO:199; or (i) a Vα CDR1-encoding polynucleotide according to SEQ ID NO:204, a Vα CDR2-encoding polynucleotide according to SEQ ID NO:203, a Vα CDR3-encoding polynucleotide according to SEQ ID NO:202, a Vβ CDR1-encoding polynucleotide according to SEQ ID NO:207, a Vβ CDR2-encoding polynucleotide according to SEQ ID NO:206, and Vβ CDR3-encoding polynucleotide according to SEQ ID NO:205.

In any of the herein disclosed embodiments, an isolated polynucleotide encoding a binding protein according to the present disclosure comprises: a Vα-encoding polynucleotide comprising or consisting of a nucleotide sequence having at least 80% identity to any one of SEQ ID NOs:230, 232, 234, 236, 238, 240, 242, 244, and 246, and a Vβ-encoding polynucleotide comprising or consisting of a nucleotide sequence having at least 80% identity to any one of SEQ ID NOs:229, 231, 233, 235, 237, 239, 241, 243, 245, and 247.

In particular embodiments, an isolated polynucleotide encoding a binding protein according to the present disclosure comprises: (a) a Vα-encoding polynucleotide comprising or consisting of the nucleotide sequence according to SEQ ID NO:230, and a Vβ-encoding polynucleotide comprising or consisting of the nucleotide sequence according to SEQ ID NO:231; (b) a Vα-encoding polynucleotide comprising or consisting of the nucleotide sequence according to SEQ ID NO:232, and a Vβ-encoding polynucleotide comprising or consisting of the nucleotide sequence according to SEQ ID NO:233; (c) a Vα-encoding polynucleotide comprising or consisting of the nucleotide sequence according to SEQ ID NO:234, and a Vβ-encoding polynucleotide comprising or consisting of the nucleotide sequence according to SEQ ID NO:235; (d) a Vα-encoding polynucleotide comprising or consisting of the nucleotide sequence according to SEQ ID NO:236, and a Vβ-encoding polynucleotide comprising or consisting of the nucleotide sequence according to SEQ ID NO:237; (e) a Vα-encoding polynucleotide comprising or consisting of the nucleotide sequence according to SEQ ID NO:238, and a Vβ-encoding polynucleotide comprising or consisting of the nucleotide sequence according to SEQ ID NO:239; (f) a Vα-encoding polynucleotide comprising or consisting of the nucleotide sequence according to SEQ ID NO:240, and a Vβ-encoding polynucleotide comprising or consisting of the nucleotide sequence according to SEQ ID NO:241; (g) a Vα-encoding polynucleotide comprising or consisting of the nucleotide sequence according to SEQ ID NO:242, and a Vβ-encoding polynucleotide comprising or consisting of the nucleotide sequence according to SEQ ID NO:243; (h) a Vα-encoding polynucleotide comprising or consisting of the nucleotide sequence according to SEQ ID NO:244, and a Vβ-encoding polynucleotide comprising or consisting of the nucleotide sequence according to SEQ ID NO:245; or (i) a Vα-encoding polynucleotide comprising or consisting of the nucleotide sequence according to SEQ ID NO:246, and a Vβ-encoding polynucleotide comprising or consisting of the nucleotide sequence according to SEQ ID NO:247.

In certain further embodiments, an isolated polynucleotide encoding a binding protein according to the present disclosure further comprises: (a) a Ca-domain-encoding polynucleotide, wherein the Vα-domain-encoding polynucleotide and the Ca-domain-encoding polynucleotide together comprise a TCR α-chain-encoding polynucleotide; and/or (b) a Cβ-domain-encoding polynucleotide, wherein the Vβ-domain-encoding polynucleotide and the Cβ-domain-encoding polynucleotide together comprise a TCR β-chain-encoding polynucleotide.

In certain embodiments, a Cα-domain-encoding polynucleotide comprises a polynucleotide having at least 80% identity to SEQ ID NO:251. In certain embodiments, a Cα-domain-encoding polynucleotide comprises or consists of a polynucleotide of SEQ ID NO:251.

In any of the embodiments described herein, a binding protein-encoding polynucleotide can further comprise a polynucleotide that encodes a self-cleaving polypeptide, wherein the polynucleotide encoding the self-cleaving polypeptide is located between, for example, a polynucleotide encoding a V_{α} chain and a polynucleotide encoding a V_{β} chain. When the binding protein encoding polynucleotides and self-cleaving polypeptide are expressed by a host cell, the binding protein will be present on the host cell surface as separate molecules that can associate or form a complex (e.g., TCR). In certain embodiments, a self-cleaving polypeptide comprises a 2A peptide from porcine teschovirus-1 (P2A; SEQ ID NO:259, encoded by, for example, the polynucleotide of SEQ ID NO:254 or 255), Thosea asigna virus (T2A; SEQ ID NO:260, encoded, for example, by the polynucleotide of SEQ ID NO:256), equine rhinitis A virus (E2A; SEQ ID NO:261, encoded by, for example, the polynucleotide of SEQ ID NO:257), or foot-and-mouth disease virus (F2A; SEQ ID NO:262, encoded by, for example, the polynucleotide of SEQ ID NO:258). Further exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety). In certain embodiments, a polynucleotide encoding a self-cleaving peptide is disposed between the TCR α-chain encoding polynucleotide and the TCR β-chain encoding polynucleotide, wherein the polynucleotide construct will have a TCR α-chain-2A-TCR β-chain structure or the polynucleotide construct will have a TCR TCR α-chain structure.

In particular embodiments, an isolated polynucleotide encoding a binding protein of the present disclosure comprises a nucleotide sequence as set forth in any one of SEQ ID NOs.: 266-274.

In further embodiments, a modified immune cell of the present disclosure comprises a heterologous polynucleotide encoding a CD8 co-receptor or an extracellular portion thereof. In certain embodiments, CD8α chain-encoding polynucleotide of the present disclosure comprises or consists of a polynucleotide having at least 80% identity to the nucleotide sequence set forth in SEQ ID NO:296. In certain embodiments, a CD8β chain-encoding polynucleotide of the present disclosure comprises or consists of the nucleotide sequence set forth in SEQ ID NO:297. In any of the presently disclosed embodiments, a modified immune cell may comprise a heterologous polynucleotide encoding a CD8α chain and a CD8β chain (or extracellular portions thereof), wherein a polynucleotide encoding a self-cleaving peptide is disposed between the polynucleotide encoding the CD8α chain and the polynucleotide encoding the CD8β chain. In some embodiments, an encoded self-cleaving peptide comprises or consists of the amino acid sequence set forth in any one of SEQ ID NOs:259-262. In further embodiments, a polynucleotide encoding the self-cleaving peptide comprises or consists of the nucleotide sequence set forth in any one of SEQ ID NOs:254-258.

In particular embodiments, a CD8 co-receptor-encoding polynucleotide comprises or consists of, in a 5′ to 3′ direction, ([a CD8α chain-encoding polynucleotide]−[a self-cleaving peptide-encoding polynucleotide]−[a CD8β chain-encoding polynucleotide]). In certain embodiments, a CD8-co-receptor-encoding polynucleotide comprises or consists of the nucleotide sequence set forth in SEQ ID NO:298.

In other embodiments, a CD8 co-receptor-encoding polynucleotide comprises or consists of, in a 5′ to 3′ direction, ([a CD8β chain-encoding polynucleotide]−[a self-cleaving peptide-encoding polynucleotide]−[a CD8α chain-encoding polynucleotide]). In certain embodiments, a CD8-co-receptor-encoding polynucleotide comprises or consists of the nucleotide sequence set forth in SEQ ID NO:299.

In further embodiments, the CD8 co-receptor-encoding polynucleotide comprises a polynucleotide encoding a self-cleaving polypeptide. In certain embodiments, the CD8 co-receptor-encoding polynucleotide comprises or consists of the nucleotide sequence set forth in SEQ ID NO:298. In certain embodiments, the CD8 co-receptor-encoding polynucleotide comprises or consists of the nucleotide sequence set forth in SEQ ID NO:299.

The present disclosure also provides any of the polynucleotide described herein contained in a vector. An exemplary vector may comprise a nucleic acid molecule capable of transporting another nucleic acid molecule to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector)). Additionally, some vectors are capable of directing the expression of genes to which they are operatively linked (these vectors may be referred to as “expression vectors”).

According to related embodiments, it is further understood that, if one or more products of interest are encoded by different polynucleotides (e.g., polynucleotides encoding binding proteins or high affinity TCRs specific for Merkel cell polyomavirus T antigen, or variants thereof, as described herein) and the products of interest are co-administered to a subject, that each polynucleotide may reside in separate vector or may reside in the same vector, and multiple vectors (each containing a different polynucleotide the same agent) may be introduced to a cell or cell population (e.g., ex vivo) for administration to a subject or directly administered to a subject.

In certain embodiments, nucleic acid molecules encoding binding proteins or high affinity TCRs specific for a Merkel cell polyomavirus T antigen epitope or peptide, may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest. In certain embodiments, polynucleotides encoding binding proteins of the instant disclosure are contained in an expression vector that is a viral vector, such as a lentiviral vector or a γ-retroviral vector.

### Host Cells

In particular embodiments, a genetically engineered expression vector is introduced into an appropriate host cell, for example, an immune cell such as a T cell or an antigen-presenting cell, i.e., a cell that displays a peptide/MHC complex on its cell surface (e.g., a dendritic cell) and lacks CD8. In certain embodiments, a host cell is a hematopoietic progenitor cell or a human immune system cell. For example, an immune system cell can be a CD4+ T cell, a CD8+ T cell, a CD4− CD8− double negative T cell, a γδ T cell, a natural killer cell, a natural killer T cell, a dendritic cell, or any combination thereof. In certain embodiments, a T cell is the host cell of interest, where in the T cell can be a naïve T cell, a central memory T cell, an effector memory T cell, a stem cell memory T cell, or any combination thereof. An expression vector genetically engineered to contain a polynucleotide of this disclosure may also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al., Mol. Cell. Biol. 12:1043, 1992); Todd et al., J. Exp. Med. 177:1663, 1993); Penix et al., J. Exp. Med. 178:1483, 1993).

In any of the foregoing embodiments, a host cell that comprises a heterologous polynucleotide encoding a binding protein is an immune cell which is modified to reduce or eliminate expression of one or more endogenous genes that encode a polypeptide product selected from PD-1, LAG-3, CTLA4, TIM3, TIGIT, an HLA molecule, a TCR molecule, CD200R, Cbl-b (see, e.g., Hooper et al., Blood 132:338 (2018)) or any component or combination thereof.

Without wishing to be bound by theory, certain endogenously expressed immune cell proteins may downregulate the immune activity of a modified immune host cell (e.g., PD-1, LAG-3, CTLA4, TIGIT, CD200R, Cbl-b), or may compete with a heterologous binding protein of the present disclosure for expression by the host cell, or may interfere with the binding activity of a heterologously expressed binding protein of the present disclosure and interfere with the immune host cell binding to a target cell, or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and allows universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, CD200R, Cbl-b, an HLA component (e.g., a gene that encodes an α1 macroglobulin, an α2 macroglobulin, an α3 macroglobulin, a (31 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013); the gene editing techniques, compositions, and adoptive cell therapies of which are incorporated herein by reference in their entirety).

As used herein, the term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety. Primers useful for designing a lentivirus that expresses a CRISPR/Cas9 system for inhibiting an endogenously expressed immune cell protein include for example, primer pairs comprising forward and reverse primers having the nucleotide sequences set forth in SEQ ID NOS: and 276 and 277, 278 and 279, 280 and 281, and 282 and 283.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, CD200R, Cbl-b, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a host cell is a human hematopoietic progenitor cell transduced with a heterologous or exogenous nucleic acid molecule encoding a TCRα chain, TCRβ chain or both, wherein the TCR produced by the cell is specific for a Merkel cell polyomavirus T antigen peptide.

In some embodiments, a host cell of the present disclosure comprises a modified immune cell. In further embodiments, a modified immune cell of the present disclosure further comprises a heterologous polynucleotide encoding a CD8 co-receptor or an extracellular portion thereof. The amino acid sequence of the human CD8α includes SEQ ID NO:290, and the amino acid sequences of CD8β chain isoforms 1-5 are set forth in SEQ ID NOs: 291-295, respectively.

In certain embodiments, a host cell or modified immune cell comprises a polynucleotide encoding a binding protein specific for a Merkel cell polyomavirus T antigen and a polynucleotide encoding a CD8 co-receptor, wherein the encoded binding protein is capable of specifically binding to a Merkel cell polyomavirus T antigen peptide:HLA complex on a cell surface, and the encoded binding protein comprises: (a) a TCR Vα domain having the CDR3 amino acid sequence of SEQ ID NO:1 (the Vα CDR3 optionally encoded by the polynucleotide of SEQ ID NO:148) and a TCR Vβ domain, (b) a TCR Vβ domain having the CDR3 amino acid sequence of SEQ ID NO:4 (the Vβ CDR3 optionally encoded by the polynucleotide of SEQ ID NO:151), and a TCR Vα domain, or (c) a TCR Vα domain having the CDR3 amino acid sequence of SEQ ID NO:1 (the Vα CDR3 optionally encoded by the polynucleotide of SEQ ID NO:148) and a TCR Vβ domain and a TCR Vβ domain having the CDR3 amino acid sequence of SEQ ID NO:4 (the Vβ CDR3 optionally encoded by the polynucleotide of SEQ ID NO:151); and wherein the encoded CD8 co-receptor comprises: (a) a CD8 co-receptor α chain comprising or consisting of the amino acid sequence of SEQ ID NO: 291 (the CD8 co-receptor α chain optionally encoded by the polynucleotide of SEQ ID NO:296), and a CD8 co-receptor β chain, (b) a CD8 co-receptor β chain comprising or consisting of the amino acid sequence of any one of SEQ ID NOS:291-295 (the CD8 co-receptor β chain optionally encoded by the polynucleotide sequence set forth in SEQ ID NO:297), and a CD8 co-receptor α chain, or (c) a CD8 co-receptor α chain comprising or consisting of the amino acid sequence of SEQ ID NO: 291 (the CD8 co-receptor α chain optionally encoded by the polynucleotide of SEQ ID NO:296), and a CD8 co-receptor β chain comprising or consisting of the amino acid sequence of any one of SEQ ID NOS:291-295 (the CD8 co-receptor β chain optionally encoded by the polynucleotide sequence set forth in SEQ ID NO:297).

In further embodiments, an encoded binding protein of this disclosure comprises a Vα CDR1 comprising or consisting of the amino acid sequence set forth in SEQ ID NO:3 (optionally encoded by the nucleotide sequence set forth in SEQ ID NO:150), a Vα CDR2 comprising or consisting of the amino acid sequence set forth in SEQ ID NO:2 (optionally encoded by the nucleotide sequence set forth in SEQ ID NO:149), a Vβ CDR1 comprising or consisting of the amino acid sequence set forth in SEQ ID NO:6 (optionally encoded by the nucleotide sequence set forth in SEQ ID NO:153), and a Vβ CDR2 comprising or consisting of the amino acid sequence set forth in SEQ ID NO:5 (optionally encoded by the nucleotide sequence set forth in SEQ ID NO:152). In particular embodiments, the encoded Vα domain comprises or consists of the amino acid sequence set forth in SEQ ID NO:63 (optionally encoded by the nucleotide sequence set forth in SEQ ID NO:228). In particular embodiments, the encoded Vβ domain comprises or consists of the amino acid sequence set forth in SEQ ID NO:64 (optionally encoded by the nucleotide sequence set forth in SEQ ID NO:229).

In certain embodiments, an encoded binding protein of this disclosure comprises a Vα CDR3 comprising or consisting of the amino acid sequence set forth in SEQ ID NO:61, and a Vβ CDR3 comprising or consisting of the amino acid sequence set forth in SEQ ID NO:62. In particular embodiments, the encoded Vα domain comprises or consists of the amino acid sequence set forth in SEQ ID NO:83 (optionally encoded by a polynucleotide as set forth in SEQ ID NO:248), and the encoded Vβ domain comprises or consists of the amino acid sequence set forth in SEQ ID NO:84 (optionally encoded by a polynucleotide as set forth in SEQ ID NO:249).

In certain embodiments, a modified host cell is provided that comprises a heterologous polynucleotide encoding a binding protein, wherein the encoded binding comprises (a) a T cell receptor (TCR) α chain variable (Vα) domain having a CDR3 amino acid sequence according to SEQ ID NO.:1 or 61, and a TCR β chain variable (Vβ) domain;

(b) a Vβ domain having a CDR3 amino acid sequence according to any one of SEQ ID NOS.:4 or 62, and a Vα domain; or (c) a Vα domain having a CDR3 amino acid sequence according to any one of SEQ ID NOS:1 or 61, and a Vβ domain having a CDR3 amino acid sequence according to any one of SEQ ID NOs:4 or 62; and wherein the binding protein is capable of specifically binding to a Merkel cell polyomavirus T antigen peptide:HLA complex on a cell surface, and wherein the modified immune cell comprises a chromosomal gene knockout of a PD-1 gene; a LAG3 gene; a TIM3 gene; a CBLB gene, a CD200R gene, a CTLA4 gene; an HLA component gene; a TCR component gene, or any combination thereof. In certain embodiments, the modified immune cell is a T cell, optionally a CD4+ T cell, a CD8+ T cell, or both.

In further embodiments, the modified immune cell comprises a chromosomal gene knockout of a PD-1 gene; a CBLB gene; a CD200R gene, or any combination thereof. In still further embodiments, the modified immune cell comprises a chromosomal gene knockout of a PD-1 gene, a CBLB gene, and a CD200R gene.

In still further embodiments, the encoded Vα domain comprises a CDR3 amino acid sequence according to SEQ ID NO:1 and the encoded Vβ domain comprises a CDR3 amino acid sequence according to SEQ ID NO:4. In further embodiments, the encoded Vα domain further comprises a CDR1 amino acid sequence according to SEQ ID NO:3 and a CDR2 amino acid sequence according to SEQ ID NO:2, and the encoded Vβ domain further comprises a CDR1 amino acid sequence according to SEQ ID NO:6 and a CDR2 amino acid sequence according to SEQ ID NO:5.

In certain embodiments, the encoded Vα domain comprises or consists of an amino acid sequence having at least 85%, 90%, 91%, 92%, 93%, 94%, 95%, 96%, 97%, 98%, 99%, or more, identity to the amino acid sequence set forth in SEQ ID NO:63, and/or wherein the encoded Vβ domain comprises or consists of an amino acid sequence having at least 85%, 90%, 91%, 92%, 93%, 94%, 95%, 96%, 97%, 98%, 99%, or more, identity to the amino acid sequence set forth in SEQ ID NO:64.

In other embodiments, the encoded Vα domain comprises a CDR3 amino acid sequence according to SEQ ID NO:61 and the encoded Vβ domain comprises a CDR3 amino acid sequence according to SEQ ID NO:62. In further embodiments, the encoded Vα domain comprises or consists of an amino acid sequence having at least 85%, 90%, 91%, 92%, 93%, 94%, 95%, 96%, 97%, 98%, 99%, or more, identity to the amino acid sequence set forth in SEQ ID NO:83, and/or the wherein encoded Vβ domain comprises or consists of an amino acid sequence having at least 85%, 90%, 91%, 92%, 93%, 94%, 95%, 96%, 97%, 98%, 99%, or more, identity to the amino acid sequence set forth in SEQ ID NO:84. In certain embodiments, the encoded binding protein comprises a Vα CDR1, a Vα CDR2, a Vβ CDR1, and Vβ CDR2 according to TCR1072 (i.e., as determined according to the amino acid sequences set forth in SEQ ID NOs:83 and 84).

In certain embodiments, the modified immune cell further comprises a heterologous polynucleotide encoding a CD8 co-receptor or an extracellular portion thereof.

It will be further understood that a host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

### Methods of Treatment

In certain aspects, the instant disclosure is directed to methods for treating a hyperproliferative or proliferative disorder or a condition characterized by Merkel cell polyomavirus T antigen expression by administering to a human subject in need thereof a composition comprising a binding protein or high affinity TCR specific for Merkel cell polyomavirus T antigen according to any the binding proteins or TCRs described herein.

The presence of a hyperproliferative or proliferative disorder or malignant condition in a subject refers to the presence of dysplastic, cancerous and/or transformed cells in the subject, including, for example neoplastic, tumor, non-contact inhibited or oncogenically transformed cells, or the like (e.g., Merkel cell carcinoma). In certain embodiments, there are provided methods for treating a Merkel cell carcinoma.

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary). In general, an appropriate dose and treatment regimen provide one or more of a binding protein or high affinity TCR specific for a Merkel cell polyomavirus T antigen epitope or peptide, or a host cell expressing such a binding protein or high affinity TCR, and optionally in combination with an adjunctive therapy (e.g., a cytokine such as IL-2, IL-15, IL-21, or any combination thereof; chemotherapy such as interferon-beta (IFN-β), radiation therapy such as localized radiation therapy), in an amount sufficient to provide therapeutic or prophylactic benefit. Therapeutic or prophylactic benefit resulting from therapeutic treatment or prophylactic or preventative methods include, for example an improved clinical outcome, wherein the object is to prevent or retard or otherwise reduce (e.g., decrease in a statistically significant manner relative to an untreated control) an undesired physiological change or disorder, or to prevent, retard or otherwise reduce the expansion or severity of such a disease or disorder. Beneficial or desired clinical results from treating a subject include abatement, lessening, or alleviation of symptoms that result from or are associated the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; or overall survival.

“Treatment” can also mean prolonging survival when compared to expected survival if a subject were not receiving treatment. Subjects in need of the methods and compositions described herein include those who already have the disease or disorder, as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit, as described in the examples.

Cells expressing a binding protein or high affinity TCR specific for a Merkel cell polyomavirus T antigen epitope or peptide as described herein may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human mammalian subject.

A therapeutically effective dose is an amount of host cells (expressing a binding protein or high affinity TCR specific for a Merkel cell polyomavirus T antigen epitope or peptide) used in adoptive transfer that is capable of producing a clinically desirable result (i.e., a sufficient amount to induce or enhance a specific T cell immune response against cells expressing a Merkel cell polyomavirus T antigen (e.g., a cytotoxic T cell (CTL) response in vivo or cell lysis in vitro in the presence of the specific Merkel cell polyomavirus T antigen epitope or peptide) in a statistically significant manner) in a treated human or non-human mammal. As is well known in the medical arts, the dosage for any one patient depends upon many factors, including the patient's size, weight, body surface area, age, the particular therapy to be administered, sex, time and route of administration, general health, and other drugs being administered concurrently. Doses will vary, but a preferred dose for administration of a host cell comprising a recombinant expression vector as described herein is about 10⁶ cells/m², about 5×10⁶ cells/m² about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

Unit doses are also provided herein which comprise a host cell (e.g., a modified immune cell comprising a polynucleotide of the present disclosure) or host cell composition of this disclosure. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of modified CD45RA⁻CD3⁺CD8⁺ and modified CD45RA⁻CD3⁺CD4⁺ T_{M} cells.

In any of the embodiments described herein, a unit dose comprises equal, or approximately equal numbers of modified CD4+ CD25− T cells and modified CD8+ CD62L+ T cells.

Also contemplated are pharmaceutical compositions that comprise binding proteins or cells expressing the binding proteins as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

A condition associated with Merkel cell polyomavirus T antigen expression includes any disorder or condition in which cellular or molecular events lead to hyperproliferative disorder, such as Merkel cell carcinoma (MCC). A subject having such a disorder or condition would benefit from treatment with a composition or method of the presently described embodiments. Some conditions associated with Merkel cell polyomavirus T antigen expression may include acute as well as chronic or recurrent disorders and diseases, such as those pathological conditions that predispose a subject to MCC.

Certain methods of treatment or prevention contemplated herein include administering a host cell (which may be autologous, allogeneic or syngeneic) comprising a desired nucleic acid molecule as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a Merkel cell polyomavirus T antigen-targeted T-cell composition to a subject as an adoptive immunotherapy.

As used herein, administration of a composition or therapy or combination therapies thereof refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., Merkel cell polyomavirus T antigen specific recombinant (i.e., engineered) host cells with one or more cytokines, such as IL-2; immunosuppressive therapy such as a chemotherapy (e.g., IFN-β, etoposide, carboplatin), radiation therapy (e.g., localized), surgical excision, Mohs micrographic surgery, immune modulators (e.g., immune modulators, such as immune checkpoint inhibitors, including antibodies specific for PD-1, PD-L1, CTLA-4), or any combination thereof), or a treatment that upregulates MHC Class I, such as localized radiation (e.g., single fraction irradiation is well accepted as a treatment for metastatic MCC palliation or single fraction radiation therapy targeting 8Gy is used on a single MCC lesion; see, e.g., Iyer et al., Cancer Med. 4:1161, 2015), one or more Th1-type cytokines (e.g., IFN-β, IFN-γ), or any combination thereof.

In still further embodiments, the subject being treated may further receive other immunosuppressive therapy, such as calcineurin inhibitors, corticosteroids, microtubule inhibitors, low dose of a mycophenolic acid prodrug, or any combination thereof. In yet further embodiments, a subject being treated has received a non-myeloablative or a myeloablative cellular immnunotherapy transplant, wherein the treatment may be administered at least two to at least three months after the non-myeloablative or myeloablative cell transplant.

In certain embodiments, a plurality of doses of a modified host cell as described herein is administered to the subject, which may be administered at intervals between administrations of about two to about four weeks. In further embodiments, a cytokine is administered sequentially, provided that the subject was administered the modified cell at least three or four times before cytokine administration. In certain embodiments, the cytokine is administered subcutaneously (e.g., IL-2, IL-15, IL-21). In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

In some embodiments, compositions and host cells as described herein are administered with chemotherapeutic agents or immune modulators (e.g., immunosuppressants, or inhibitors of immunosuppression components, such as immune checkpoint inhibitors). Immune checkpoint inhibitors include inhibitors of CTLA-4, A2AR, B7-H3, B7-H4, BTLA, HVEM, GAL9, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. An inhibitor of an immune checkpoint molecule can be an antibody or antigen binding fragment thereof, a fusion protein, a small molecule, an RNAi molecule, (e.g., siRNA, shRNA, or miRNA), a ribozyme, an aptamer, or an antisense oligonucleotide. A chemotherapeutic can be a B-Raf inhibitor, a MEK inhibitor, a VEGF inhibitor, a VEGFR inhibitor, a tyrosine kinase inhibitor, an anti-mitotic agent, or any combination thereof.

In any of the embodiments herein, a method of treating a subject having or at risk of having Merkel cell carcinoma, comprising administering to human subject having or at risk of having Merkel cell carcinoma a composition comprising a binding protein specific for a Merkel cell polyomavirus T antigen peptide as disclosed herein, and a therapeutically effective amount of an inhibitor of an immunosuppression component, such as an immune checkpoint inhibitor. In some embodiments, an immune checkpoint inhibitor is an inhibitor of CTLA-4, A2AR, B7-H3, B7-H4, BTLA, HVEM, GAL9, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. In further embodiments, the instant disclosure provides a method of treating a subject having or at risk of having Merkel cell carcinoma, comprising administering to human subject having or at risk of having Merkel cell carcinoma a composition comprising (a) a binding protein specific for a Merkel cell polyomavirus T antigen peptide as disclosed herein, (b) a therapeutically effective amount of an inhibitor of an immunosuppression component, such as an immune checkpoint inhibitor, and (c) an upregulator of MHC Class I molecules, such as localized radiation (e.g., single fraction irradiation), IFN-β, IFN-γ, or a combination thereof.

Accordingly, in certain embodiments, this disclosure provides methods of treating a subject having or at risk of having Merkel cell carcinoma, comprising administering to a subject having or at risk of having Merkel cell carcinoma a therapeutically effective amount of a modified immune cell, composition, or unit dose of the present disclosure, and a therapeutically effective amount of an inhibitor of an immunosuppression component, such as an immune checkpoint inhibitor. In some embodiments, an immune checkpoint inhibitor is an inhibitor of CTLA-4, A2AR, B7-H3, B7-H4, BTLA, HVEM, GAL9, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. In some embodiments, an immune checkpoint inhibitor is selected from (a) an antibody specific for PD-1, such as pidilizumab, lambrolizumab, nivolumab, or pembrolizumab; (b) an antibody specific for PD-L1, such as avelumab, BMS-936559 (also known as MDX-1105), durvalumab, or atezolizumab; or (c) an antibody specific for CTLA4, such as tremelimumab or ipilimumab. In any of these methods, the treatment may further comprise an upregulator of WIC Class I molecules, such as localized radiation (e.g., single fraction irradiation), IFN-β, IFN-γ, or a combination thereof.

In further embodiments, this disclosure provides methods of treating a subject having or at risk of having Merkel cell carcinoma, comprising administering to a subject having or at risk of having Merkel cell carcinoma a therapeutically effective amount of a modified immune cell, composition, or unit dose of the present disclosure; and a therapeutically effective amount of an inhibitor of an immunosuppression component, such as an immune checkpoint inhibitor. In some embodiments, an immune checkpoint inhibitor is an inhibitor of CTLA-4, A2AR, B7-H3, B7-H4, BTLA, HVEM, GAL9, IDO, KIR, LAG-3, PD-1, PD-L1, PD-L2, Tim-3, VISTA, TIGIT, LAIR1, CD160, 2B4, TGFR beta, CEACAM-1, CEACAM-3, CEACAM-5, CD244, or any combination thereof. In some embodiments, an immune checkpoint inhibitor is selected from (a) an antibody specific for PD-1, such as pidilizumab, lambrolizumab, nivolumab, or pembrolizumab; (b) an antibody specific for PD-L1, such as BMS-936559 (also known as MDX-1105), durvalumab, atezolizumab, or avelumab; or (c) an antibody specific for CTLA4, such as tremelimumab or ipilimumab.

In certain embodiments, a binding protein of the present disclosure (or a modified host cell expressing the same) is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a binding protein of the present disclosure (or a modified host cell expressing the same) is used in combination with an inhibitor of CD244.

In certain embodiments, a binding protein of the present disclosure (or a modified host cell expressing the same) is used in combination with an inhibitor of BLTA, HVEM, CD160, or any combination thereof. Anti CD-160 antibodies are described in, for example, PCT Publication No. WO 2010/084158.

In certain embodiments, a binding protein of the present disclosure (or a modified host cell expressing the same) is used in combination with an inhibitor of TIM3.

In certain embodiments, a binding protein of the present disclosure (or a modified host cell expressing the same) is used in combination with an inhibitor of Gal9.

In certain embodiments, a binding protein of the present disclosure (or a modified host cell expressing the same) is used in combination with an inhibitor of adenosine signaling, such as a decoy adenosine receptor.

In certain embodiments, a binding protein of the present disclosure (or a modified host cell expressing the same) is used in combination with an inhibitor of A2aR.

In certain embodiments, a binding protein of the present disclosure (or a modified host cell expressing the same) is used in combination with an inhibitor of KIR, such as lirilumab (BMS-986015).

In certain embodiments, a binding protein of the present disclosure (or a modified host cell expressing the same) is used in combination with an inhibitor of an inhibitory cytokine (typically, a cytokine other than TGFβ) or Treg development or activity.

In certain embodiments, a binding protein of the present disclosure (or a modified host cell expressing the same) is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a binding protein of the present disclosure (or a modified host cell expressing the same) is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a binding protein of the present disclosure (or a modified host cell expressing the same) is used in combination with an inhibitor of VISTA, such as CA-170 (Curis, Lexington, Mass.).

In certain embodiments, a binding protein of the present disclosure (or a modified host cell expressing the same) is used in combination with an inhibitor of TIGIT such as, for example, COM902 (Compugen, Toronto, Ontario Canada), an inhibitor of CD155, such as, for example, COM701 (Compugen), or both.

In certain embodiments, a binding protein of the present disclosure (or a modified host cell expressing the same) is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a binding protein of the present disclosure (or a modified host cell expressing the same) is used in combination with a LAIR1 inhibitor.

In certain embodiments, a binding protein of the present disclosure (or a modified host cell expressing the same) is used in combination with an inhibitor of CEACAM-1, CEACAM-3, CEACAM-5, or any combination thereof.

In certain embodiments, a binding protein of the present disclosure (or a modified host cell expressing the same) is used in combination with an agent that increases the activity (i.e., is an agonist) of a stimulatory immune checkpoint molecule. For example, a fusionprotein of the present disclosure (or an engineered host cell expressing the same) can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, Cβ-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof).

In any of the embodiments disclosed herein, a method may comprise administering a binding protein of the present disclosure (or a modified host cell expressing the same) with one or more agonist of a stimulatory immune checkpoint molecule, including any of the foregoing, singly or in any combination.

In certain embodiments, a combination therapy comprises a binding protein of the present disclosure (or a modified host cell expressing the same) and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a fusion protein and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

Exemplary chemotherapeutic agents include alkylating agents (e.g., cisplatin, oxaliplatin, carboplatin, busulfan, nitrosoureas, nitrogen mustards such as bendamustine, uramustine, temozolomide), antimetabolites (e.g., aminopterin, methotrexate, mercaptopurine, fluorouracil, cytarabine, gemcitabine), taxanes (e.g., paclitaxel, nab-paclitaxel, docetaxel), anthracyclines (e.g., doxorubicin, daunorubicin, epirubicin, idaruicin, mitoxantrone, valrubicin), bleomycin, mytomycin, actinomycin, hydroxyurea, topoisomerase inhibitors (e.g., camptothecin, topotecan, irinotecan, etoposide, teniposide), monoclonal antibodies (e.g., ipilimumab, pembrolizumab, nivolumab, avelumab, alemtuzumab, bevacizumab, cetuximab, gemtuzumab, panitumumab, rituximab, tositumomab, trastuzumab), vinca alkaloids (e.g., vincristine, vinblastine, vindesine, vinorelbine), cyclophosphamide, prednisone, leucovorin, oxaliplatin, hyalurodinases, or any combination thereof. In certain embodiments, a chemotherapeutic is vemurafenib, dabrafenib, trametinib, cobimetinib, sunitinib, erlotinib, paclitaxel, docetaxel, or any combination thereof. In some embodiments, a patient is first treated with a chemotherapeutic agent that inhibits or destroys other immune cells followed by a pharmaceutical composition described herein. In some cases, chemotherapy may be avoided entirely.

Cytokines are used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described Merkel cell polyomavirus T antigen-specific binding proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a Merkel cell polyomavirus T antigen-derived peptide as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a modified cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of modified cells or active compound calculated to produce the desired therapeutic effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

### EXAMPLES

### Example 1

### Materials and Methods

Human Subjects and Samples:

This study was approved by the Fred Hutchinson Cancer Research Center (FHCRC) Institutional Review Board and conducted according to Declaration of Helsinki principles. Informed consent was received from all participants. Subjects were HLA class I typed via polymerase chain reaction (PCR) at Bloodworks Northwest (Seattle, Wash.). PBMC: Heparinized blood was obtained from healthy donors and peripheral blood mononuclear cells (PBMCs) were cryopreserved after routine Ficoll preparation at a dedicated specimen processing facility at FHCRC.

T Cell Receptor β Sequencing and Analysis:

Tetramer+ Cells: At least 2 million PBMC or TIL were stained with anti-CD8-PE antibody (Clone 3B5, Life Technologies), A*02/KLL-APC tetramer (Immune Monitoring Lab, FHCRC) and 7-AAD viability dye (BioLegend). Tetramer+, CD8^{high} cells were sorted via FACSAriaII (BD) and flash frozen (average of 710 cells from PBMC (n=9), 5776 cells from TIL (n=5), range 350-8,000 and 1844-12799, respectively). Samples were submitted to Adaptive Biotechnologies (Seattle, Wash.) for genomic DNA extraction, TRB sequencing and normalization. All TRB sequences detected in ≥2 cells (estimated number of genomes ≥2) were categorized as tetramer+ clonotypes.

Creation of KLL-Specific T Cell Clones:

PBMC were stained and sorted as described above into T cell medium (TCM) containing RPMI, 8% human serum, 200 nM L-glutamine and 100 U/ml Penicillin-Streptomycin, and cloned at 0.25 to 3 cells per well with allogeneic irradiated feeders, IL-2 (Hemagen Diagnostics) and PHA (Remel) as described²⁹ with addition of 20 ng/mL rIL-15 (R&D Systems) after day 2. After 2 weeks, microcultures with visible growth were screened for specificity via tetramer; TCR variable beta chain (TCRVβ) expression was assessed by staining clones with fluorescent anti-TCRVβ antibodies (IOTest Beta Mark, Beckman Coulter). Wells selected for screening, expansion, and TCR analysis came from plates with <37% of cultures having visual growth, yielding a 95% chance of clonality per the Poisson distribution (Chen et al., J. Immunol. Methods 52:307, 1982). Cultures with tetramer+ cells, reactivity to peptide and dissimilar TCRVβ chains were further expanded with IL-2 and anti-CD3 clone OKT3 mAb (Miltenyi Biotec) as described in Iyer et al., 2011, plus 20 ng/mL rIL-15. Prior to harvesting RNA for TCR analysis, cultures were held at least 2 weeks to minimize persistent feeder cell-derived RNA. CD8-independent Tetramer Staining: Clones were stained with a HLA-A*02:01/KLL tetramer containing D227K/T228A mutations in HLA-A*02:01, using methods as above. These mutations abrogate HLA class I:CD8 binding to identify clones expressing TCRs with the ability to bind independent of CD8 stabilization and can indicate high TCR avidity (Choi et al., J. Immunol. 171:5116, 2003; Laugel et al., J. Biol. Chem. 282:23799, 2007).

TCR α & β Sequencing of Clones:

Simultaneous sequencing of TCRα and TCRβ repertoires was performed as described in Han et al., Nat. Biotechnol. 32:684, 2014. Briefly, total RNA was isolated from clonally expanded populations using Qiagen RNeasy Plus, followed by One Step RT/PCR (Qiagen) primed with multiplexed TCR primers. This reaction was used as template with a second set of nested TCRα and TCRβ primers, followed by PCR to add barcoding and paired end primers. Templates were purified using AMPure (Agencourt Biosciences) then normalized prior to running on Illumina MiSeq v2-300. Pairs of 150 nucleotide sequences were merged into contigs using PandaSeq (Masella et al., BMC Bioinformatics 13:31, 2012). Merged sequences were then separated according to inline barcodes identifying the plate and well of origin, generating one file of derived sequences for each clone of interest. Files for each clone were processed with MiXCR (Bolotin et al., Nat. Methods 12:380, 2015) to identify and quantify clonotypes and assign VDJ allele usage. Cultures in which the dominant TCRβ nucleotide sequence was present at <97% of productive sequence reads were classified as possibly polyclonal and excluded from further analysis.

T Cell Functional Assays:

T cell clones were tested for specificity and functional avidity via cytokine release assays. Cytokine Release with Peptide-pulsed Targets: Secreted IFN-γ was measured after co-incubating 2×10⁴ clonal KLL-specific T cells with 5×10⁴ T2 cells (ATCC) plus antigenic peptide at log₁₀ dilutions to final concentration from 10⁻⁶ to 10⁻¹² molar in 200 μl TCM for 36 hours. Due to possible oxidation and dimerization of cysteine residues in the antigenic KLLEIAPNC (SEQ ID NO:284) peptide, the homolog KLLEIAPNA (SEQ ID NO:285) was used to allow for efficient HLA class I presentation; similar substitution has been shown to not alter recognition of HLA-peptide complex by TCRs raised against the native peptide (Webb et al., J. Biol. Chem. 279:23438, 2004). IFN-γ in cell culture supernatants was assayed by ELISA according to manufacturer's recommendations (Human IFN gamma ELISA Ready-SET-Go Kit, affymetrix). To estimate EC₅₀ (the amount of peptide leading to 50% of maximum IFN-γ secretion), IFN-γ secretion by each T cell clone was analyzed via nonlinear regression using Prism version 6.0 (GraphPad). In addition, IFN-γ release by KLL-specific clonotypes was measured after incubation with three MCPyV+, HLA-A*02+ MCC cell lines (WaGa and MKL-2 [gift of Dr. Becker, German Cancer Research Center], and MS-1 [gift of Dr. Shuda, University of Pittsburgh]. Cell lines were early passage and authenticated with short tandem repeat analysis). Cell lines were stimulated with IFN-β (Betaseron, Bayer Health Care; 3,000 U/mL) for 24 hours to induce expression of HLA class I, followed by 24 hours of culture after IFN-β washout. A total of 2×10⁴ clonal KLL-specific T cells were incubated with 5×10⁴ cells from each MCC cell line, +/−IFN-β treatment, and incubated for 36 hours. Supernatants were assayed by ELISA as described above. Cytokine Release with Large T-Ag transfected Targets: T cell clones were incubated with antigen presenting cells transiently transfected with plasmids encoding HLA-A*02:01 and GFP-truncated Large T-Ag (tLTAg) fusion protein (pDEST103-GFP-tLTAg). pDEST103-GFP-tLTAg was created using Gateway recombination cloning technology (Gateway) to insert tLTAg from pCMV-MCV156 (Paulson et al., Cancer Res. 70:8388, 2010) into pDEST103-GFP. A total of 3×10⁴COS-7 cells (ATCC, CRL-1651) were plated in flat-bottom 96-well plates in DMEM+10% FBS, 200 nM L-glutamine and 100 U/ml Penicillin-Streptomycin. After incubating for 24 hours, wells were transfected using FuGENE HD (Promega) at a 6:1 ratio of transfection reagent to DNA with 25 ng HLA-A*02:01 and limiting dilution of pDEST103-GFP-tLTAg (25-0.08 ng) plus irrelevant DNA (pcDNA-6/myc-His C, Gateway) to a total of 25 ng. 48 hours after transfection, 10⁴ viable KLL-specific T cells in TCM were added to target wells in duplicate. After 36 hours, supernatants were assayed by ELISA for IFN-γ secretion and EC₅₀ calculated as above. Transfected COS-7 cells were harvested at 48 and 72 hours post-transfection to quantitate transfection efficiency by flow cytometry.

T Cell Receptor Clonality:

Tetramer-sorted cells: Shannon entropy was calculated on the estimated number of genomes (≥2) of all productive TRB and normalized by dividing by the log 2 of unique productive sequences in each sample. Clonality was calculated as 1−normalized entropy.

### Example 2

### Functional Testing of MCPyV-LT Antigen-Specific TCRs

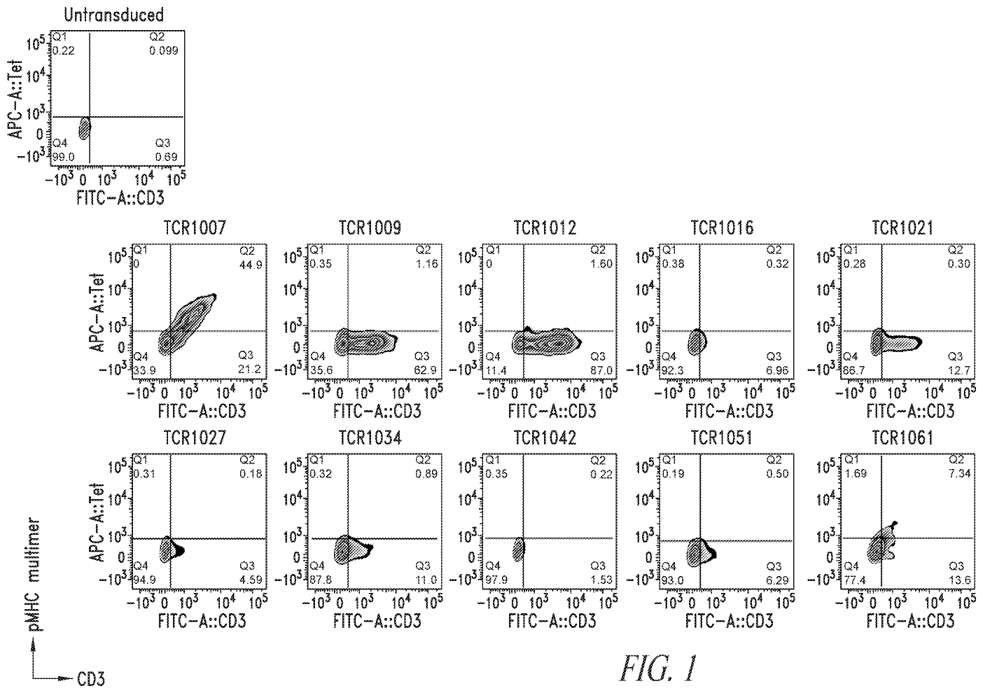

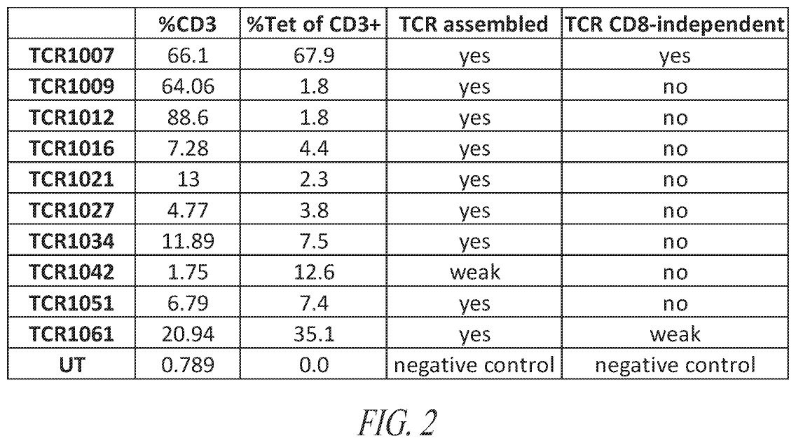

Ten (10) donor-derived TCRs were examined for the ability to bind MCPyV Large-T Antigen-presenting pMHC multimers in the absence of the CD8 co-receptor. CD8^{-/-} Jurkat or T cells were transduced with codon-optimized polynucleotides encoding the TCRs and cells were stained for multimers and for CD3 expression. Data are shown in FIG. 1 and summarized in FIG. 2. TCR1007 demonstrated the most robust responses and was selected for further testing.

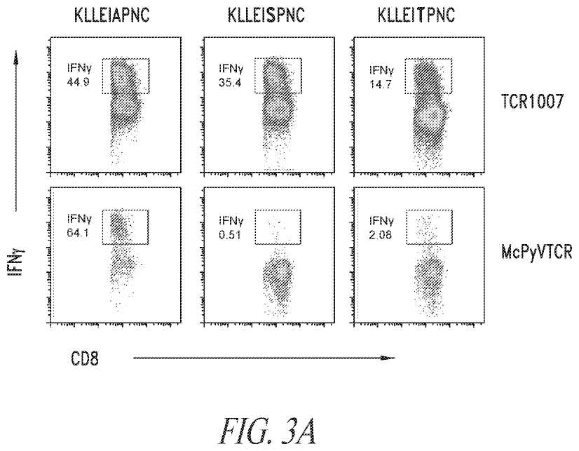

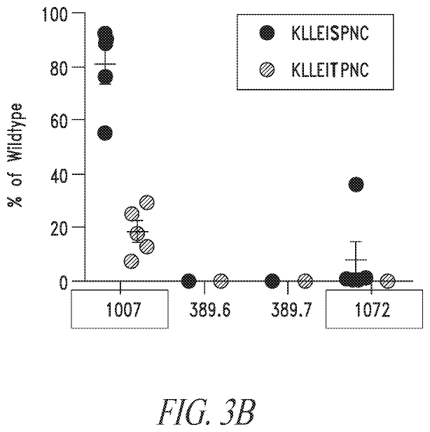

The ability of TCR1007-transduced T cells to efficiently recognize the MCPyV Large-T antigen and close sequence variants thereof, and to produce cytokines in response to antigen, was tested. Transduced T cells (4 donors) were incubated overnight with antigen-presenting cells (APCs) loaded with Larg-T peptide (or variant) antigens (5 ug/mL). As shown in FIG. 3A, TCR1007-transduced cells produced interferon-gamma (IFN-γ) when co-cultured with APCs presenting any of the peptide antigens. MCPyV TCR=comparator MCC patient-derived TCR. The percentage of TCR1007-transduced cells recognizing the MCPyV Large-T sequence variants KLLEISPNC (SEQ ID NO:286) and KLLEITPNC (SEQ ID NO:287) was quantified, as shown in FIG. 3B. “389.6”, “389.7”, and “TCR1072” are cells transduced with comparator MCC patient-derived TCRs.

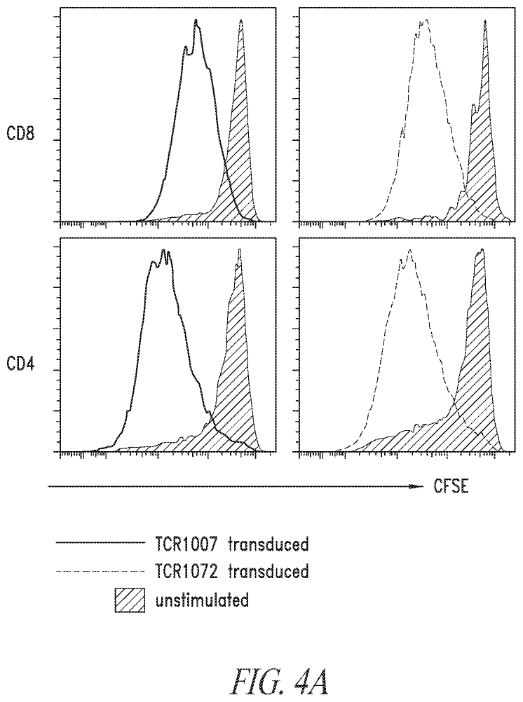

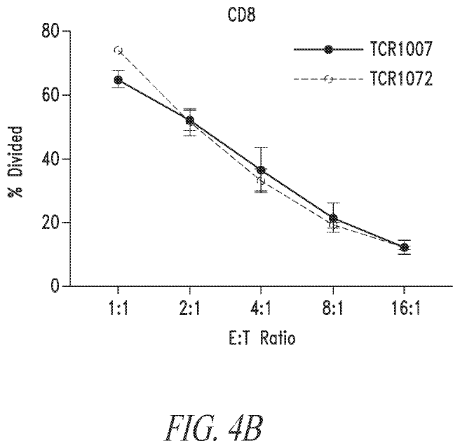

To investigate the ability of the MCPyV-specific TCRs to stimulate T cell proliferation in response to antigen, CD8+ and CD4+ T cells (4 donors) were transduced with TCR1007 or TCR1072 and stimulated with antigen-presenting irradiated fibroblasts in culture. As shown in FIG. 4A, both types of T cell proliferated in response to endogenously presented antigen (day 6 CFSE dilution followed by flow cytometry). FIG. 4B shows that a high percentage of TCR1007- and TCR1072-transduced CD8 T cells underwent at least one division in co-culture with target cells at a 1:1 ratio.

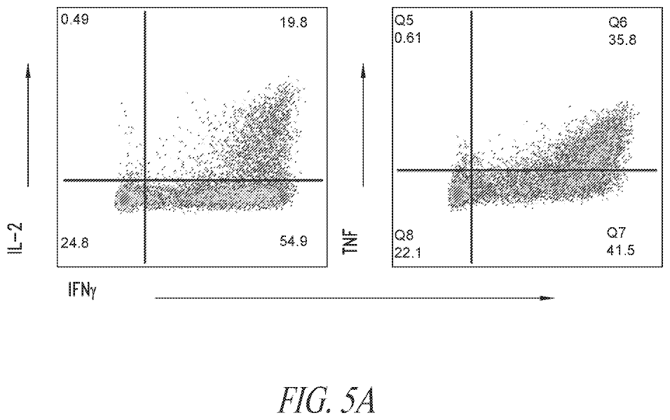

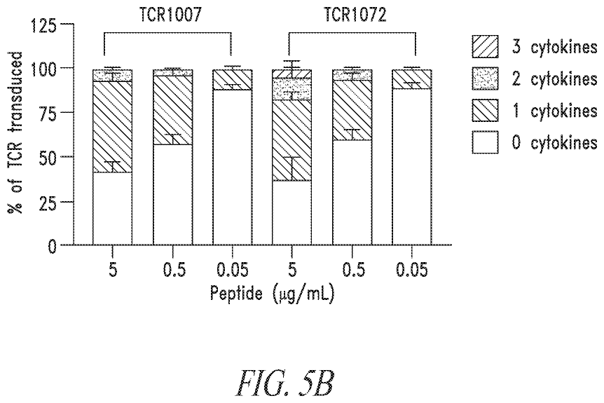

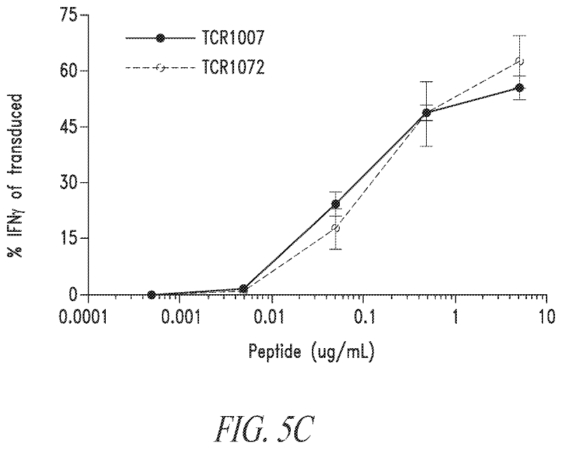

The ability of MCPyV-specific TCR-transduced cells to produce cytokines (IL-2, TNFα) was also investigated. As shown in FIG. 5A, TCR1007-transduced CD8 T cells (3 donors) effectively produced both cytokines in the presence of endogenous IFN-γ. The percentage of donor CD8 T cells transduced with TCR1007 or TCR1072 that produced one or more cytokines was also determined (FIG. 5B). Transduced T cells also secreted IFN-γ when co-cultured with APCs loaded with increasing levels of antigen (FIG. 5C).

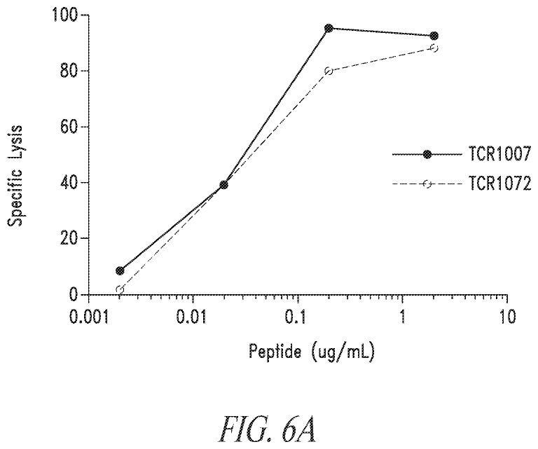

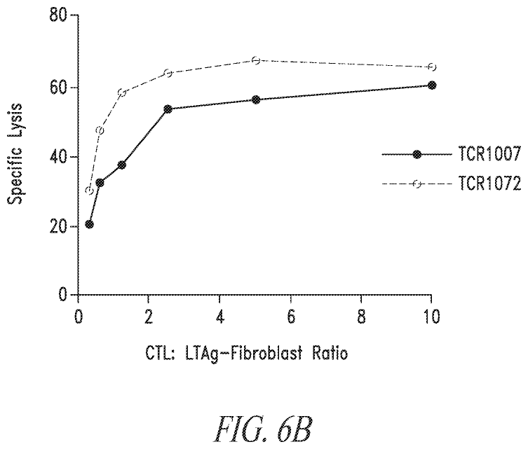

Next, the ability of MCPyV-specific T cells to specifically kill target APCs was tested in a standard 4 h Cr⁵¹-release assay. As shown in FIG. 6A, both TCR1007- and TCR1072-transduced CD8 T cells (1 donor) specifically killed APCs in a peptide dose-dependent manner. Lytic activity at various effector:target cell ratios is shown in FIG. 6B.

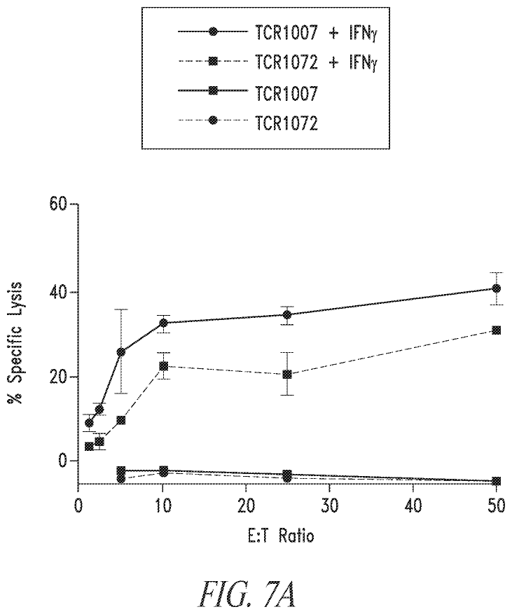

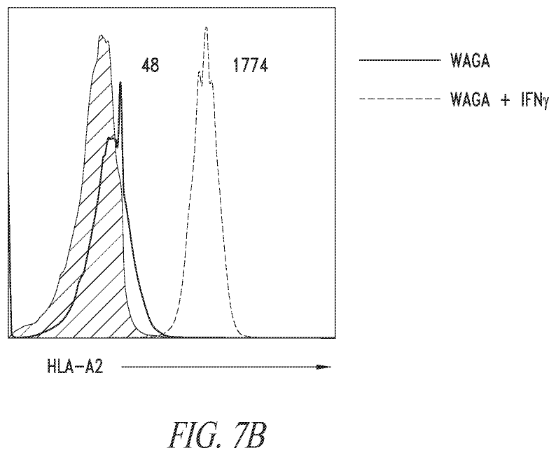

MCPyV-specific CD8 T cells (3 donors) also specifically killed antigen-presenting cancer cells (WAGA cell line) in a 72-hour co-culture, but required the addition of exogenous IFN-γ, as shown in FIG. 7A. HLA-A2 expression by the WAGA cells correlated with the presence of IFN-γ, as shown in FIG. 7B.

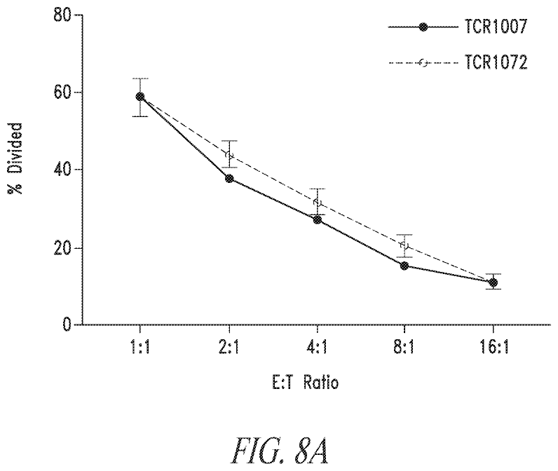

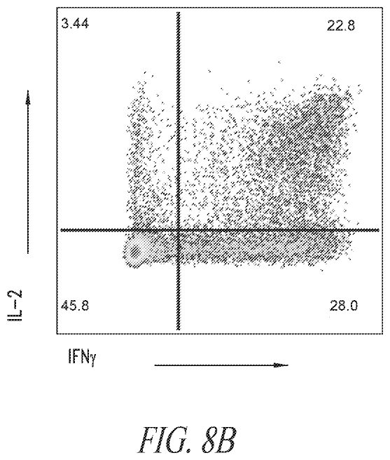

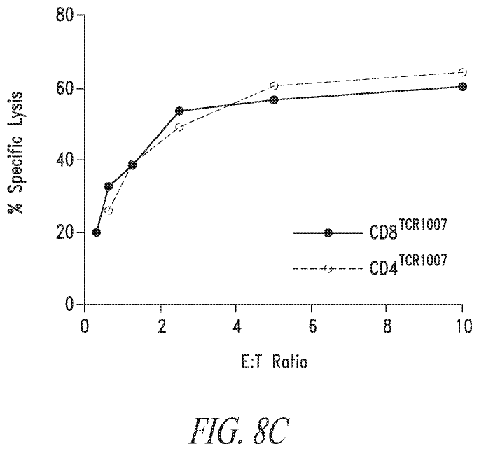

The ability of MCPyV-specific TCRs to engage CD4+ T cells was also investigated. As shown in FIG. 8A, CD4+ T cells (3 donors) transduced with TCR1007 or TCR1072 underwent cell division in co-culture with APCs, albeit at a somewhat lower percentage of the T cells as compared to CD8 T cells (see FIG. 4B). TCR1007-transduced CD4+ T cells also produced cytokine (IL-2) in response to stimulation with antigen (FIG. 8B). Notably, CD4+ T cells transduced with TCR1007 had similar specific killing activity against antigen-presenting target cells as CD8+ transduced T cells (FIG. 8C).

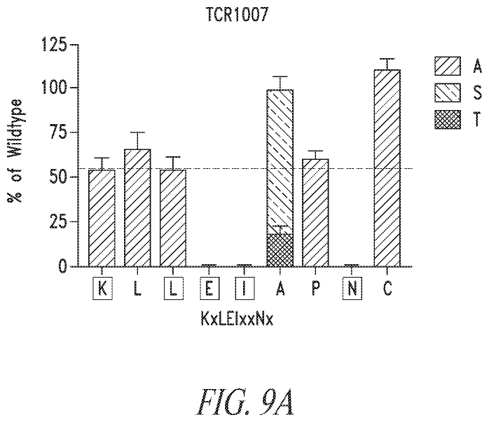

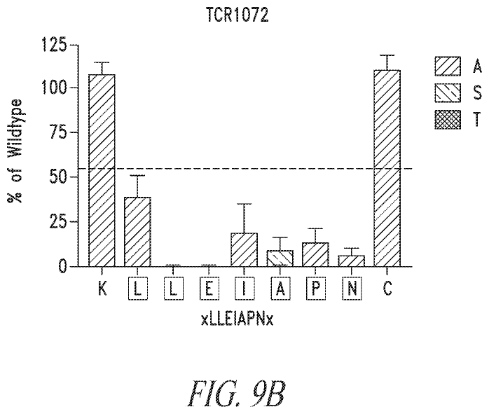

A candidate TCR for use in immunotherapy to treat MCC should not only be able to efficiently recognize and kill MCPyV-expressing cancer cells, but should have minimal or no off-target effects. To investigate the likelihood that MCPyV-specific TCRs might bind undesired targets (i.e., expressed on healthy tissues) the consensus residues required for recognition by TCR1007 and TCR1072 were determined by alanine scanning mutagenesis. As shown in FIG. 9A, TCR1007 requires the consensus sequence KxLEIxxNx (SEQ ID NO:288) to recognize the Large-T antigen. TCR1072 requires the consensus sequence xLLEIAPNx (SEQ ID NO:289).

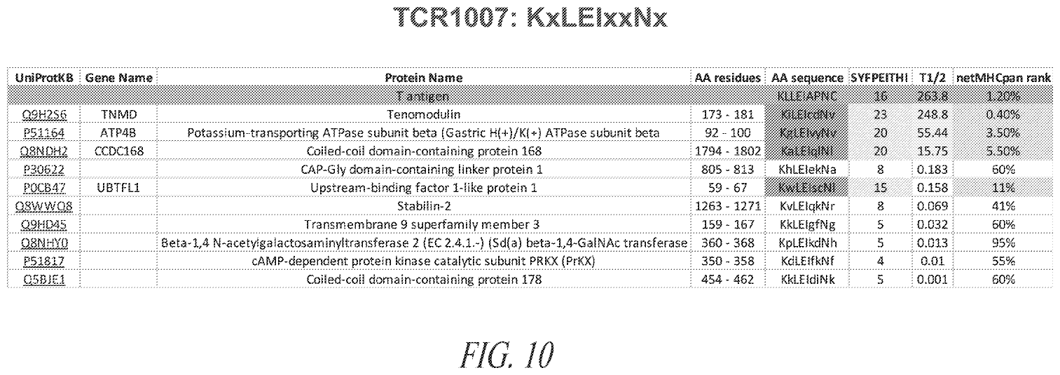

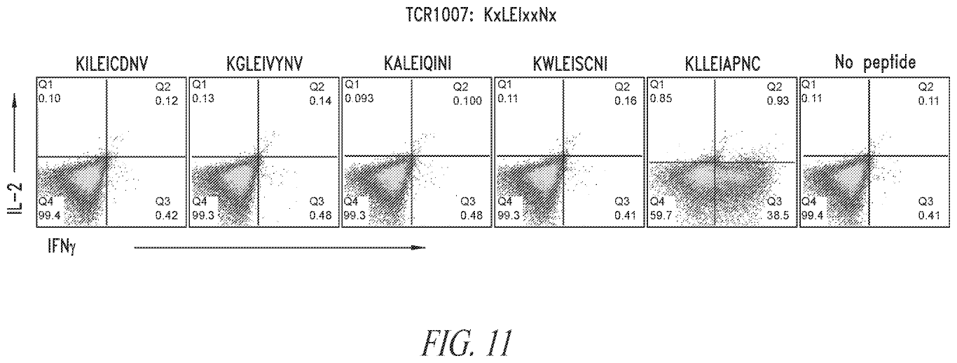

The human proteome was then interrogated for peptides with high sequence homology to SEQ ID NO:288 (FIG. 10). Four peptides were selected for further testing. As shown in FIG. 11, donor CD8 T cells transduced with TCR1007 produced IL-2 in response to APCs expressing the Large-T antigen (KLLEIAPNC SEQ ID NO: 284), but not in response to APCs expressing the normal human peptides.

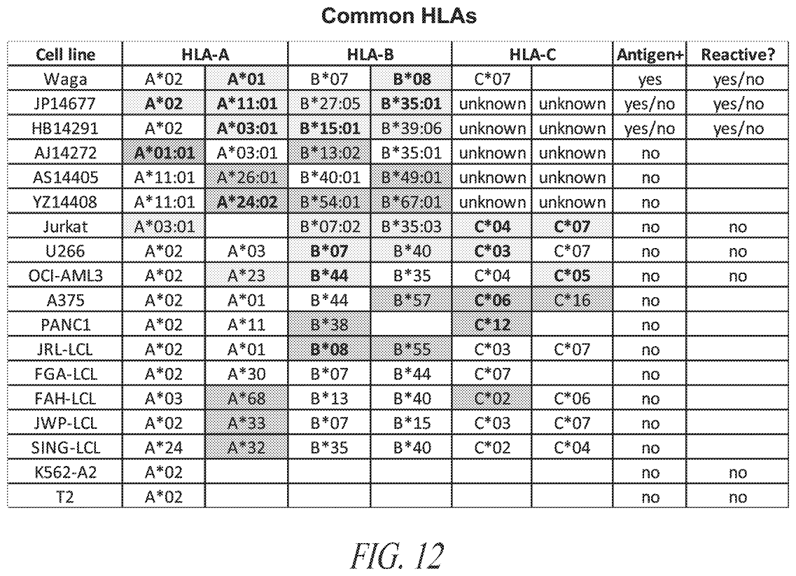

In ongoing studies, various HLA alleles are investigated for potential cross-reactivity with TCR1007. Data are provided in FIG. 12.

### Example 3

### Clinical Study of T Cell Therapy for Merkel Cell Carcinoma

A Phase I clinical study of T cell therapy for treating Merkel Cell Carcinoma (MCC) is conducted. Two infusions of autologous T cells expressing MCC antigen-specific TCRs are administered to patients: (1) 100 million (10⁸) MCPyV-specific TCR transgenic CD8+ T cells in an initial low dose phase I infusion, and 1 billion (10⁹) MCPyV-specific TCR transgenic CD8+ T cells in a full dose infusion. All patients included in the study are adults and have similar body surface areas.

The cell product includes both CD8+ and CD4+ transgenic T cells. Doses are measured based on the quantity of transgenic CD8+ cells. A 1:1 ratio of CD8+ and CD4+ transgenic T cells is targeted, though there may be variability in infused products. Therefore, all transgenic CD4+ T cells generated to reach the CD8+ dose are be included. The total transgenic cell dose including both CD8+ and CD4+ T cells allowed is 10 times the targeted CD8+ dose (10¹⁰ transgenic T cells for full dose infusion and 10⁹ transgenic T cells for initial infusion for the first three patients). The maximum combined dose is <5% and <50% of the maximum previously infused safe dose for endogenous CD8+ therapy for MCC for the dose escalation and full dose infusions, respectively.

Briefly, leukapheresis product is obtained from each patient. CD4+ T cells are enriched by positive immunomagnetic selection using GMP compliant Clinimacs reagent systems on the Clinimacs Prodigy instrument (Miltenyi). From the flow-through (containing CD8+ T cells), CD62L+ cells are enriched by positive immunomagnetic selection. The enriched cells are combined in approximately 1:1 ratio. The enriched cells are activated with GMP T Cell TransAct (Milteyni) and transduced with lentiviral vector supernatant on day 1 after stimulation. Each of the T cell subsets is then expanded in media supplemented with interleukin-2 (IL-2). Cells are harvested at the end of the culture, counted, and washed. Cells for the first infusion are infused fresh unless there is a clinical contraindication; cells for the second infusion are formulated in cryopreservation media and cryopreserved.

The T cell therapy may depend on antigen presentation by the primary tumor through class I MHC. It has been shown that approximately 80% of MCC tumors downregulate MHC-I, presenting an obstacle to T cell efficacy; this is has been reported for tumors that have escaped immunotherapy. However, class I downregulation is typically reversible with one of several interventions, including low dose single fraction radiation therapy (SFRT). For this study, SFRT is administered to a single tumor lesion to enhance tumor visibility by MHC class I upregulation, to ‘prime’ tumor prior to T cell infusion, and to palliate the treated lesion. An additional measurable lesion is left untreated to assess systemic efficacy.

Avelumab (anti-PD-L1) is front-line systemic therapy for metastatic MCC and is currently the only FDA-approved agent in this setting. All patients enrolled in the trial will have had disease progression on or after treatment with a PD-1 axis checkpoint inhibitor, such as avelumab. Avelumab therapy will be administered beginning at least 2 weeks after T cell infusion to promote persistence and reduce exhaustion of transferred MCPyV-specific T cells.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/672,232 filed May 16, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A modified immune cell comprising a heterologous polynucleotide encoding a binding protein, wherein the encoded binding protein comprises:
(a) a T cell receptor (TCR) α chain variable (Vα) domain having a CDR3 amino acid sequence according to any one of SEQ ID NOS.:7, 13, 19, 25, 31, 37, 43, 49, and 55, and a TCR β chain variable (Vβ) domain;
(b) a Vβ domain having a CDR3 amino acid sequence according to any one of SEQ ID NOS.:10, 16, 22, 28, 34, 40, 46, 52, and 58, and a Vα domain; or
(c) a Vα domain having a CDR3 amino acid sequence according to any one of SEQ ID NOS:7, 13, 19, 25, 31, 37, 43, 49, and 55, and a Vβ domain having a CDR3 amino acid sequence according to any one of SEQ ID NOs:10, 16, 22, 28, 34, 40, 46, 52, and 58; and
wherein the binding protein is capable of specifically binding to a Merkel cell polyomavirus T antigen peptide:HLA complex on a cell surface.

2. The modified immune cell according to claim 1, wherein the encoded binding protein is capable of specifically binding a KLLEIAPNC (SEQ ID NO:284):human leukocyte antigen (HLA) complex or a KLLEIAPNA (SEQ ID NO:285):human leukocyte antigen (HLA) complex.

3. The modified immune cell according to claim 1 or 2, wherein the V_{β} domain of (a) is derived from V, D, and J alleles according to Table 1.

4. The modified immune cell according to any one of claims 1-3, wherein the V_{α} domain of (b) is derived from V and J alleles according to Table 1.

5. The modified immune cell according to any one of claims 1-4, wherein the encoded binding protein comprises a V_{α} domain that is at least about 90% identical to an amino acid sequence of SEQ ID NO: 65, 67, 69, 71, 73, 75, 77, 79, or 81 and comprises a V_{β} domain that is at least about 90% identical to an amino acid sequence of SEQ ID NO: 66, 68, 70, 72, 74, 76, 78, 80, or 82, provided that (a) at least three or four of the CDRs have no change in sequence, wherein the CDRs that do have sequence changes have only up to two amino acid substitutions, up to a contiguous five amino acid deletion, or a combination thereof, and (b) the binding protein remains capable of specifically binding to a Merkel cell polyomavirus T antigen peptide:HLA cell surface complex.

6. The modified immune cell according to any one of claims 1-5, wherein:
(a) the encoded Vα domain comprises (i) a CDR1 amino acid sequence according to any one of SEQ ID NOS:9, 15, 21, 27, 33, 39, 45, 51, and 57, and/or (ii) a CDR2 amino acid sequence according to any one of SEQ ID NOS:8, 14, 20, 26, 32, 38, 44, 50, and 56; and/or
(b) the encoded Vβ domain comprises (iii) a CDR1 amino acid sequence according to any one of SEQ ID NOS:12, 18, 24, 30, 36, 42, 48, 54, and 60, and/or (iv) a CDR2 amino acid sequence according to any one of SEQ ID NOS:11, 17, 23, 29, 35, 41, 47, 53, and 59.

7. The modified immune cell according to any one of claims 1-6, wherein the encoded binding protein comprises:
(a) Vα CDR1, CDR2, and CDR3 amino acid sequences according to SEQ ID NOS:9, 8, and 7, respectively, and Vβ CDR1, CDR2, and CDR3 amino acid sequences according to SEQ ID NOS:12, 11, and 10, respectively;
(b) Vα CDR1, CDR2, and CDR3 amino acid sequences according to SEQ ID NOS:15, 14, and 13, respectively, and Vβ CDR1, CDR2, and CDR3 amino acid sequences according to SEQ ID NOS:18, 17, and 16, respectively;
(c) Vα CDR1, CDR2, and CDR3 amino acid sequences according to SEQ ID NOS:21, 20, and 19, respectively, and Vβ CDR1, CDR2, and CDR3 amino acid sequences according to SEQ ID NOS:24, 23, and 22, respectively;
(d) Vα CDR1, CDR2, and CDR3 amino acid sequences according to SEQ ID NOS:27, 26, and 25, respectively, and Vβ CDR1, CDR2, and CDR3 amino acid sequences according to SEQ ID NOS:30, 29, and 28, respectively;
(e) Vα CDR1, CDR2, and CDR3 amino acid sequences according to SEQ ID NOS:33, 32, and 31, respectively, and Vβ CDR1, CDR2, and CDR3 amino acid sequences according to SEQ ID NOS:36, 35, and 34, respectively;
(f) Vα CDR1, CDR2, and CDR3 amino acid sequences according to SEQ ID NOS:39, 38, and 37, respectively, and Vβ CDR1, CDR2, and CDR3 amino acid sequences according to SEQ ID NOS:42, 41, and 40, respectively;
(g) Vα CDR1, CDR2, and CDR3 amino acid sequences according to SEQ ID NOS:45, 44, and 43, respectively, and Vβ CDR1, CDR2, and CDR3 amino acid sequences according to SEQ ID NOS:48, 47, and 46, respectively;
(h) Vα CDR1, CDR2, and CDR3 amino acid sequences according to SEQ ID NOS:51, 50, and 49, respectively, and Vβ CDR1, CDR2, and CDR3 amino acid sequences according to SEQ ID NOS:54, 53, and 52, respectively; or
(i) Vα CDR1, CDR2, and CDR3 amino acid sequences according to SEQ ID NOS:57, 56, and 55, respectively, and Vβ CDR1, CDR2, and CDR3 amino acid sequences according to SEQ ID NOS:60, 59, and 58, respectively.

8. The modified immune cell according to any one of claims 1-7, wherein the encoded binding protein specifically binds to a KLLEIAPNC (SEQ ID NO:284):HLA-A*201 complex.

9. The modified immune cell according to any one of claims 1-8, wherein the encoded Vα domain comprises or consists of an amino acid sequence according to SEQ ID NO.: 65, 67, 69, 71, 73, 75, 77, 79, or 81.

10. The modified immune cell according to any one of claims 1-9, wherein the encoded Vβ domain comprises or consists of an amino acid sequence according to SEQ ID NO.: 66, 68, 70, 72, 74, 76, 78, 80, or 82.

11. The modified immune cell of claim 10, wherein:
(a) the encoded Vα domain comprises or consists of the amino acid sequence according to SEQ ID NO.:65 and the encoded Vβ domain comprises or consists of the amino acid sequence according to SEQ ID NO.:66;
(b) the encoded Vα domain comprises or consists of the amino acid sequence according to SEQ ID NO.:67 and the encoded Vβ domain comprises or consists of the amino acid sequence according to SEQ ID NO.:68;
(c) the encoded Vα domain comprises or consists of the amino acid sequence according to SEQ ID NO.:69 and the encoded Vβ domain comprises or consists of the amino acid sequence according to SEQ ID NO.:70;
(d) the encoded Vα domain comprises or consists of the amino acid sequence according to SEQ ID NO.:71 and the encoded Vβ domain comprises or consists of the amino acid sequence according to SEQ ID NO.:72;
(e) the encoded Vα domain comprises or consists of the amino acid sequence according to SEQ ID NO.:73 and the encoded Vβ domain comprises or consists of the amino acid sequence according to SEQ ID NO.:74;
(f) the encoded Vα domain comprises or consists of the amino acid sequence according to SEQ ID NO.:75 and the encoded Vβ domain comprises or consists of the amino acid sequence according to SEQ ID NO.:76;
(g) the encoded Vα domain comprises or consists of the amino acid sequence according to SEQ ID NO.:77 and the encoded Vβ domain comprises or consists of the amino acid sequence according to SEQ ID NO.:78;
(h) the encoded Vα domain comprises or consists of the amino acid sequence according to SEQ ID NO.:79 and the encoded Vβ domain comprises or consists of the amino acid sequence according to SEQ ID NO.:80; or
(i) the encoded Vα domain comprises or consists of the amino acid sequence according to SEQ ID NO.:81 and the encoded Vβ domain comprises or consists of the amino acid sequence according to SEQ ID NO.:82.

12. The modified immune cell according to any one of claims 1-10, further comprising a heterologous polynucleotide encoding a TCR α chain constant (Cα), a heterologous polynucleotide encoding a TCR β chain constant (Cβ), or both.

13. The modified immune cell according to claim 12, wherein the encoded Cα domain comprises an amino acid sequence with at least 90% sequence identity to an amino acid sequence according to SEQ ID NO.:85.

14. The modified immune cell according to claim 12 or 13, wherein the encoded Cβ domain comprises an amino acid sequence with at least 90% sequence identity to the amino acid sequence according to SEQ ID NO.:86 or 87.

15. The modified immune cell according to claim 14, wherein the encoded binding protein comprises a V_{α} domain comprising or consisting of SEQ ID NO.:65, a V_{β} domain comprising or consisting of SEQ ID NO.:66, a C_{α} domain comprising or consisting of SEQ ID NO.:85, and a C_{β} domain comprising or consisting of SEQ ID NO.:86.

16. The modified immune cell according to claim 14, wherein the encoded binding protein comprises a V_{α} domain comprising or consisting of SEQ ID NO.:67, a V_{β} domain comprising or consisting of SEQ ID NO.:68, a C_{α} domain comprising or consisting of SEQ ID NO.:85, and a C_{β} domain comprising or consisting of SEQ ID NO.:87.

17. The modified immune cell according to claim 14, wherein the encoded binding protein comprises a V_{α} domain comprising or consisting of SEQ ID NO.:69, a V_{β} domain comprising or consisting of SEQ ID NO.:70, a C_{α} domain comprising or consisting of SEQ ID NO.:85, and a C_{β} domain comprising or consisting of SEQ ID NO.:87.

18. The modified immune cell according to claim 14, wherein the encoded binding protein comprises a V_{α} domain comprising or consisting of SEQ ID NO.:71, a V_{β} domain comprising or consisting of SEQ ID NO.:72, a C_{α} domain comprising or consisting of SEQ ID NO.:85, and a C_{β} domain comprising or consisting of SEQ ID NO.:87.

19. The modified immune cell according to claim 14, wherein the encoded binding protein comprises a V_{α} domain comprising or consisting of SEQ ID NO.:73, a V_{β} domain comprising or consisting of SEQ ID NO.:74, a C_{α} domain comprising or consisting of SEQ ID NO.:85, and a C_{β} domain comprising or consisting of SEQ ID NO.:87.

20. The modified immune cell according to claim 14, wherein the encoded binding protein comprises a V_{α} domain comprising or consisting of SEQ ID NO.:75, a V_{β} domain comprising or consisting of SEQ ID NO.:76, a C_{α} domain comprising or consisting of SEQ ID NO.:85, and a C_{β} domain comprising or consisting of SEQ ID NO.:86.

21. The modified immune cell according to claim 14, wherein the encoded binding protein comprises a V_{α} domain comprising or consisting of SEQ ID NO.:77, a V_{β} domain comprising or consisting of SEQ ID NO.:78, a C_{α} domain comprising or consisting of SEQ ID NO.:85, and a C_{β} domain comprising or consisting of SEQ ID NO.:87.

22. The modified immune cell according to claim 14, wherein the encoded binding protein comprises a V_{α} domain comprising or consisting of SEQ ID NO.:79, a V_{β} domain comprising or consisting of SEQ ID NO.:80, a C_{α} domain comprising or consisting of SEQ ID NO.:85, and a C_{β} domain comprising or consisting of SEQ ID NO.:86.

23. The modified immune cell according to claim 14, wherein the encoded binding protein comprises a V_{α} domain comprising or consisting of SEQ ID NO.:81, a V_{β} domain comprising or consisting of SEQ ID NO.:82, a C_{α} domain comprising or consisting of SEQ ID NO.:85, and a C_{β} domain comprising or consisting of SEQ ID NO.:87.

24. The modified immune cell according to any one of claims 1-23, wherein the binding protein is a T cell receptor (TCR), an antigen-binding fragment of a TCR, or a chimeric antigen receptor.

25. The modified immune cell according to claim 24, wherein the TCR, the chimeric antigen receptor, or the antigen-binding fragment of the TCR is chimeric, humanized or human.

26. The modified immune cell according to claim 24 or 25, wherein the antigen-binding fragment of the TCR comprises a single chain TCR (scTCR).

27. The modified immune cell according to any one of claims 22-25, wherein the binding protein is a chimeric antigen receptor, optionally a TCR-CAR.

28. The modified immune cell according to any one of claims 24-27, wherein the binding protein is a TCR.

29. The modified immune cell according to any one of claims 1-28, wherein the modified immune cell is a human immune cell.

30. The modified immune cell according to claim 29, wherein the immune cell is a T cell, a NK cell, or a NK-T cell.

31. The modified immune cell according to claim 30, wherein the immune cell is a CD4+ T cell, a CD8+ T cell, or both.

32. The modified immune cell according to any one of claims 29-31, wherein the modified immune cell comprises a chromosomal gene knockout of a PD-1 gene; a LAG3 gene; a TIM3 gene; a CTLA4 gene; an HLA component gene; a TCR component gene, a CBLB gene, a CD200R gene, or any combination thereof.

33. The modified immune cell according to claim 32, wherein the chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene.

34. The modified immune cell according to claim 32, wherein the chromosomal gene knockout comprises a knockout of a TCR component gene selected from a TCR α variable region gene, a TCR β variable region gene, a TCR constant region gene, or a combination thereof.

35. The modified immune cell according to any one of claims 31-34, wherein the modified immune cell is a CD4+ T cell and further comprises a heterologous polynucleotide encoding at least an extracellular portion of a CD8 co-receptor.

36. The modified immune cell according to claim 35, wherein the polynucleotide encoding the binding protein and/or the polynucleotide encoding the at least an extracellular portion of a CD8 co-receptor is codon-optimized for expression by the modified immune cell.

37. A composition comprising a modified immune cell according to any one of claims 1-36 and a pharmaceutically acceptable carrier, diluent, or excipient.

38. A unit dose, comprising an effective amount of (i) the modified immune cell according to any one of claims 1-36 or (ii) a composition according to claim 37.

39. The unit dose according to claim 38, comprising at least about 30% modified CD4+ T cells, combined with (ii) a composition comprising at least about 30% modified CD8+ T cells, in about a 1:1 ratio.

40. The unit dose according to claim 39, wherein the unit dose contains substantially no naïve T cells.

41. An isolated polynucleotide encoding a binding protein having a TCR Vα domain and a TCR Vβ domain, wherein the encoded binding protein is capable of specifically binding to a Merkel cell polyomavirus T antigen peptide:HLA complex on a cell surface, the isolated polynucleotide comprising:
(a) a Vα CDR3-encoding polynucleotide according to SEQ ID NO:154, 160, 166, 172, 178, 184, 190, 196, or 202, and a Vβ-encoding polynucleotide;
(b) a Vβ CDR3-encoding polynucleotide according to SEQ ID NO:157, 163, 169, 175, 181, 187, 193, 199, or 205, and a Vα-encoding polynucleotide; or
(c) a Vα CDR3-encoding polynucleotide according to SEQ ID NO: 154, 160, 166, 172, 178, 184, 190, 196, or 202, and a Vβ CDR3-encoding polynucleotide according to SEQ ID NO: SEQ ID NO:157, 163, 169, 175, 181, 187, 193, 199, or 205.

42. The isolated polynucleotide according to claim 41, wherein the Vβ-encoding polynucleotide of (a) is derived from V, D, and J alleles according to Table 1.

43. The isolated polynucleotide according to claim 41 or 42, wherein the Vα-encoding polynucleotide of (b) is derived from V and J alleles according to Table 1.

44. The isolated polynucleotide according to any one of claims 41-43, comprising:
(a) a Vα CDR3-encoding polynucleotide according to SEQ ID NO:154 and a Vβ CDR3-encoding polynucleotide according to SEQ ID NO:157;
(b) a Vα CDR3-encoding polynucleotide according to SEQ ID NO:160 and a Vβ CDR3-encoding polynucleotide according to SEQ ID NO:163;
(c) a Vα CDR3-encoding polynucleotide according to SEQ ID NO:166 and a Vβ CDR3-encoding polynucleotide according to SEQ ID NO:169;
(d) a Vα CDR3-encoding polynucleotide according to SEQ ID NO:172 and a Vβ CDR3-encoding polynucleotide according to SEQ ID NO:175;
(e) a Vα CDR3-encoding polynucleotide according to SEQ ID NO:178 and a Vβ CDR3-encoding polynucleotide according to SEQ ID NO:181;
(f) a Vα CDR3-encoding polynucleotide according to SEQ ID NO:184 and a Vβ CDR3-encoding polynucleotide according to SEQ ID NO:187;
(g) a Vα CDR3-encoding polynucleotide according to SEQ ID NO:190 and a CDR3-encoding polynucleotide according to SEQ ID NO:193;
(h) a Vα CDR3-encoding polynucleotide according to SEQ ID NO:196 and a CDR3-encoding polynucleotide according to SEQ ID NO:199; or
(i) a Vα CDR3-encoding polynucleotide according to SEQ ID NO:202 and a Vβ CDR3-encoding polynucleotide according to SEQ ID NO:205.

45. The isolated polynucleotide according to any one of claims 41-44, further comprising:
(a) a Vα CDR1-encoding polynucleotide according to SEQ ID NO:156, 162, 168, 174, 180, 186, 192, 198, or 204;
(b) a Vα CDR2-encoding polynucleotide according to SEQ ID NO:155, 161, 167, 173, 179, 185, 191, 197, or 203;
(c) a Vβ CDR1-encoding polynucleotide according to SEQ ID NO:159, 165, 171, 177, 183, 189, 195, 201, or 207; and/or
(d) a Vβ CDR2-encoding polynucleotide according to SEQ ID NO:158, 164, 170, 176, 184, 188, 194, 200, or 206.

46. The isolated polynucleotide according to any one of claims 41-45, comprising:
(a) a Vα CDR1-encoding polynucleotide according to SEQ ID NO:156, a Vα CDR2-encoding polynucleotide according to SEQ ID NO:155, a Vα CDR3-encoding polynucleotide according to SEQ ID NO:154, a Vβ CDR1-encoding polynucleotide according to SEQ ID NO:159, a Vβ CDR2-encoding polynucleotide according to SEQ ID NO:158, and Vβ CDR3-encoding polynucleotide according to SEQ ID NO:157;
(b) a Vα CDR1-encoding polynucleotide according to SEQ ID NO:162, a Vα CDR2-encoding polynucleotide according to SEQ ID NO:161, a Vα CDR3-encoding polynucleotide according to SEQ ID NO:160, a Vβ CDR1-encoding polynucleotide according to SEQ ID NO:165, a Vβ CDR2-encoding polynucleotide according to SEQ ID NO:164, and Vβ CDR3-encoding polynucleotide according to SEQ ID NO:163;
(c) a Vα CDR1-encoding polynucleotide according to SEQ ID NO:168, a Vα CDR2-encoding polynucleotide according to SEQ ID NO:167, a Vα CDR3-encoding polynucleotide according to SEQ ID NO:166, a Vβ CDR1-encoding polynucleotide according to SEQ ID NO:171, a Vβ CDR2-encoding polynucleotide according to SEQ ID NO:170, and Vβ CDR3-encoding polynucleotide according to SEQ ID NO:169;
(d) a Vα CDR1-encoding polynucleotide according to SEQ ID NO:174, a Vα CDR2-encoding polynucleotide according to SEQ ID NO:173, a Vα CDR3-encoding polynucleotide according to SEQ ID NO:172, a Vβ CDR1-encoding polynucleotide according to SEQ ID NO:177, a Vβ CDR2-encoding polynucleotide according to SEQ ID NO:176, and Vβ CDR3-encoding polynucleotide according to SEQ ID NO:175;
(e) a Vα CDR1-encoding polynucleotide according to SEQ ID NO:180, a Vα CDR2-encoding polynucleotide according to SEQ ID NO:179, a Vα CDR3-encoding polynucleotide according to SEQ ID NO:178, a Vβ CDR1-encoding polynucleotide according to SEQ ID NO:183, a Vβ CDR2-encoding polynucleotide according to SEQ ID NO:182, and Vβ CDR3-encoding polynucleotide according to SEQ ID NO:181;
(f) a Vα CDR1-encoding polynucleotide according to SEQ ID NO:186, a Vα CDR2-encoding polynucleotide according to SEQ ID NO:185, a Vα CDR3-encoding polynucleotide according to SEQ ID NO:184, a Vβ CDR1-encoding polynucleotide according to SEQ ID NO:189, a Vβ CDR2-encoding polynucleotide according to SEQ ID NO:188, and Vβ CDR3-encoding polynucleotide according to SEQ ID NO:187;
(g) a Vα CDR1-encoding polynucleotide according to SEQ ID NO:192, a Vα CDR2-encoding polynucleotide according to SEQ ID NO:191, a Vα CDR3-encoding polynucleotide according to SEQ ID NO:190, a Vβ CDR1-encoding polynucleotide according to SEQ ID NO:194, a Vβ CDR2-encoding polynucleotide according to SEQ ID NO:193, and Vβ CDR3-encoding polynucleotide according to SEQ ID NO:192;
(h) a Vα CDR1-encoding polynucleotide according to SEQ ID NO:198, a Vα CDR2-encoding polynucleotide according to SEQ ID NO:197, a Vα CDR3-encoding polynucleotide according to SEQ ID NO:196, a Vβ CDR1-encoding polynucleotide according to SEQ ID NO:201, a Vβ CDR2-encoding polynucleotide according to SEQ ID NO:200, and Vβ CDR3-encoding polynucleotide according to SEQ ID NO:199; or
(i) a Vα CDR1-encoding polynucleotide according to SEQ ID NO:204, a Vα CDR2-encoding polynucleotide according to SEQ ID NO:203, a Vα CDR3-encoding polynucleotide according to SEQ ID NO:202, a Vβ CDR1-encoding polynucleotide according to SEQ ID NO:207, a Vβ CDR2-encoding polynucleotide according to SEQ ID NO:206, and Vβ CDR3-encoding polynucleotide according to SEQ ID NO:205.

47. The isolated polynucleotide according to claim 46, comprising:
(a) a Vα-encoding polynucleotide comprising or consisting of the nucleotide sequence according to SEQ ID NO:230, and a Vβ-encoding polynucleotide comprising or consisting of the nucleotide sequence according to SEQ ID NO:231;
(b) a Vα-encoding polynucleotide comprising or consisting of the nucleotide sequence according to SEQ ID NO:232, and a Vβ-encoding polynucleotide comprising or consisting of the nucleotide sequence according to SEQ ID NO:233;
(c) a Vα-encoding polynucleotide comprising or consisting of the nucleotide sequence according to SEQ ID NO:234, and a Vβ-encoding polynucleotide comprising or consisting of the nucleotide sequence according to SEQ ID NO:235;
(d) a Vα-encoding polynucleotide comprising or consisting of the nucleotide sequence according to SEQ ID NO:236, and a Vβ-encoding polynucleotide comprising or consisting of the nucleotide sequence according to SEQ ID NO:237;
(e) a Vα-encoding polynucleotide comprising or consisting of the nucleotide sequence according to SEQ ID NO:238, and a Vβ-encoding polynucleotide comprising or consisting of the nucleotide sequence according to SEQ ID NO:239;
(f) a Vα-encoding polynucleotide comprising or consisting of the nucleotide sequence according to SEQ ID NO:240, and a Vβ-encoding polynucleotide comprising or consisting of the nucleotide sequence according to SEQ ID NO:241;
(g) a Vα-encoding polynucleotide comprising or consisting of the nucleotide sequence according to SEQ ID NO:242, and a Vβ-encoding polynucleotide comprising or consisting of the nucleotide sequence according to SEQ ID NO:243;
(h) a Vα-encoding polynucleotide comprising or consisting of the nucleotide sequence according to SEQ ID NO:244, and a Vβ-encoding polynucleotide comprising or consisting of the nucleotide sequence according to SEQ ID NO:245; or
(i) a Vα-encoding polynucleotide comprising or consisting of the nucleotide sequence according to SEQ ID NO:246, and a Vβ-encoding polynucleotide comprising or consisting of the nucleotide sequence according to SEQ ID NO:247.

48. The isolated polynucleotide according to any one of claims 41-47, further comprising:
(a) a Cα-domain-encoding polynucleotide, wherein the Vα-domain-encoding polynucleotide and the Cα-domain-encoding polynucleotide together comprise a TCR α-chain-encoding polynucleotide; and/or
(b) a Cβ-domain-encoding polynucleotide, wherein the V β-domain-encoding polynucleotide and the Cβ-domain-encoding polynucleotide together comprise a TCR β-chain-encoding polynucleotide.

49. The isolated polynucleotide according to claim 48, wherein the Cα-domain-encoding polynucleotide comprises a polynucleotide having at least 80% identity to SEQ ID NO:251.

50. The isolated polynucleotide according to claim 49, wherein the Cα-domain-encoding polynucleotide comprises or consists of a polynucleotide of SEQ ID NO:251.

51. The isolated polynucleotide according to any one of claims 48-50, further comprising a polynucleotide encoding a self-cleaving peptide disposed between the TCR α chain-encoding polynucleotide and the TCR β chain-encoding polynucleotide.

52. The isolated polynucleotide according to claim 51, wherein the polynucleotide encoding a self-cleaving peptide comprises or consists of a nucleotide sequence according to any one of SEQ ID NOS.:254-258.

53. The isolated polynucleotide according to claim 51 or 52, wherein the polynucleotide encodes a self-cleaving peptide comprising or consisting of an amino acid sequence according to any one of SEQ ID NOS.:259-262.

54. The isolated polynucleotide according to any one of claims 51-53, comprising or consisting of the nucleotide sequence according to any one of SEQ ID NOs.:266-274.

55. An expression vector, comprising a polynucleotide according to any one of claims 41-54 operably linked to an expression control sequence.

56. The expression vector according to claim 55, wherein the vector is capable of delivering the polynucleotide to a host cell.

57. The expression vector according to claim 56, wherein the host cell is a hematopoietic progenitor cell or a human immune system cell.

58. The expression vector according to claim 57, wherein the human immune system cell is a CD4+ T cell, a CD8+ T cell, a CD4− CD8− double negative T cell, a γδ T cell, a natural killer cell, a dendritic cell, or any combination thereof.

59. The expression vector according to claim 58, wherein the T cell is a naïve T cell, a central memory T cell, an effector memory T cell, or any combination thereof.

60. The expression vector according to any one of claims 55-59, wherein the vector is a viral vector.

61. The expression vector according to claim 60, wherein the viral vector is a lentiviral vector or a γ-retroviral vector.

62. A method for treating Merkel cell carcinoma, comprising administering to human subject having or at risk of having Merkel cell carcinoma a modified immune cell of any one of claims 1-36, a composition of claim 37, or a unit dose of any one of claims 38-40.

63. The method according to claim 62, wherein the modified immune cell is capable of promoting an antigen-specific T cell response against a Merkel cell polyomavirus T antigen peptide in a class I HLA-restricted manner.

64. The method according to claim 62 or 63, wherein the class I HLA-restricted response is transporter-associated with antigen processing (TAP)-independent.

65. The method according to claim 63 or 64, wherein the antigen-specific T cell response comprises at least one of a CD4⁺ helper T lymphocyte (Th) response and a CD8+ cytotoxic T lymphocyte (CTL) response.

66. An adoptive immunotherapy method for treating a subject having a Merkel cell carcinoma, comprising administering to the subject an effective amount of a modified immune cell of any one of claims 1-36, a composition of claim 37, or a unit dose of any one of claims 38-40.

67. The method according to claim 66, wherein the modified immune cell is modified ex vivo.

68. The method according to claim 66 or 67, wherein the modified immune cell is an allogeneic cell, a syngeneic cell, or an autologous cell.

69. The method according to any one of claims 66-68, wherein the modified immune cell is a hematopoietic progenitor cell or a human immune system cell.

70. The method according to claim 69, wherein the human immune system cell is a CD4+ T cell, a CD8+ T cell, a CD4− CD8− double negative T cell, a γδ T cell, a natural killer cell, a dendritic cell, or any combination thereof.

71. The method according to claim 70, wherein the T cell is a naïve T cell, a central memory T cell, an effector memory T cell, or any combination thereof.

72. The method according to any one of claims 66-71, wherein the modified immune cell, the composition, or the unit dose is administered parenterally.

73. The method according to any one of claims 62-72, wherein the method comprises administering a plurality of doses of the modified immune cell to the subject.

74. The method according to claim 73, wherein the plurality of doses are administered at intervals between administrations of about two to about four weeks.

75. The method according to any one of claims 62-74, wherein the modified immune cell is administered to the subject at a dose of about 10⁷ cells/m² to about 10¹¹ cells/m².

76. The method according to any one of claims 62-75, wherein the method further comprises an adjunctive therapy selected from a cytokine, a chemotherapy (e.g., IFN-β, etoposide, carboplatin), radiation therapy (e.g., localized), surgical excision, Mohs micrographic surgery, immune modulators (e.g., immune modulators, such as immune checkpoint inhibitors, including antibodies specific for PD-1, PD-L1, CTLA-4), or any combination thereof.

77. The method according to any one of claims 62-76, wherein the method further comprises administering a cytokine.

78. The method according to claim 77, wherein the cytokine is IL-2, IL-15, IL-21 or any combination thereof.

79. The method according to claim 78, wherein the cytokine is IL-2 and is administered concurrently or sequentially with the modified immune cell.

80. The method according to claim 79, wherein the cytokine is administered sequentially, provided that the subject was administered the modified immune cell at least three or four times before cytokine administration.

81. The method according to any one of claims 78-80, wherein the cytokine is IL-2 and is administered subcutaneously.

82. The method according to any one of claims 62-81, wherein the subject is further receiving immunosuppressive therapy.

83. The method according to claim 82, wherein the immunosuppressive therapy is selected from calcineurin inhibitors, corticosteroids, microtubule inhibitors, low dose of a mycophenolic acid prodrug, or any combination thereof.

84. The method according to any one of claims 62-83, wherein the subject has received a non-myeloablative or a myeloablative hematopoietic cell transplant.

85. The method according to claim 84, wherein the subject is administered the modified immune cell at least three months after the non-myeloablative hematopoietic cell transplant.

86. The method according to claim 84, wherein the subject is administered the modified immune cell at least two months after the myeloablative hematopoietic cell transplant.

87. The method according to any one of claims 82-86, wherein the immunosuppressive therapy comprises (a) an antibody specific for PD-1, such as pidilizumab, lambrolizumab, nivolumab, or pembrolizumab; (b) an antibody specific for PD-L1, such as avelumab, BMS-936559 (also known as MDX-1105), durvalumab, or atezolizumab; or (c) an antibody specific for CTLA4, such as tremelimumab or ipilimumab.

88. A modified immune cell comprising a heterologous polynucleotide encoding a binding protein, wherein the encoded binding protein comprises:
(a) a T cell receptor (TCR) α chain variable (Vα) domain having a CDR3 amino acid sequence according to SEQ ID NO.:1 or 61, and a TCR β chain variable (Vβ) domain;
(b) a Vβ domain having a CDR3 amino acid sequence according to any one of SEQ ID NOS.:4 or 62, and a Vα domain; or
(c) a Vα domain having a CDR3 amino acid sequence according to any one of SEQ ID NOS:1 or 61, and a Vβ domain having a CDR3 amino acid sequence according to any one of SEQ ID NOs:4 or 62;
wherein the binding protein is capable of specifically binding to a Merkel cell polyomavirus T antigen peptide:HLA complex on a cell surface, and
wherein the modified immune cell comprises a chromosomal gene knockout of a PD-1 gene; a LAG3 gene; a TIM3 gene; a CBLB gene, a CD200R gene, a CTLA4 gene; an HLA component gene; a TCR component gene, or any combination thereof.

89. The modified immune cell of claim 88, further comprising a chromosomal gene knockout of a PD-1 gene; a CBLB gene; a CD200R gene, or any combination thereof.

90. The modified immune cell of claim 89, wherein the immune cell comprises a chromosomal gene knockout of a PD-1 gene, a CBLB gene, and a CD200R gene.

91. The modified immune cell of any one of claims 88-90, wherein the encoded Vα domain comprises a CDR3 amino acid sequence according to SEQ ID NO:1 and the encoded Vβ domain comprises a CDR3 amino acid sequence according to SEQ ID NO:4.

92. The modified immune cell of any one of claims 88-91, wherein the encoded Vα domain further comprises a CDR1 amino acid sequence according to SEQ ID NO:3 and a CDR2 amino acid sequence according to SEQ ID NO:2, and the encoded Vβ domain further comprises a CDR1 amino acid sequence according to SEQ ID NO:6 and a CDR2 amino acid sequence according to SEQ ID NO:5.

93. The modified immune cell of any one of claims 88-92, wherein the encoded Vα domain comprises or consists of an amino acid sequence having at least 85% identity to the amino acid sequence set forth in SEQ ID NO:63, and/or wherein the encoded Vβ domain comprises or consists of an amino acid sequence having at least 85% identity to the amino acid sequence set forth in SEQ ID NO:64.

94. The modified immune cell of any one of claims 88-90, wherein the encoded Vα domain comprises a CDR3 amino acid sequence according to SEQ ID NO:61 and the encoded Vβ domain comprises a CDR3 amino acid sequence according to SEQ ID NO:62.

95. The modified immune cell of claim 88 or 94, wherein the encoded Vα domain comprises or consists of an amino acid sequence having at least 85% identity to the amino acid sequence set forth in SEQ ID NO:83, and/or wherein the encoded Vβ domain comprises or consists of an amino acid sequence having at least 85%, identity to the amino acid sequence set forth in SEQ ID NO:84.

96. The modified immune cell of any one of claims 88-95, wherein the immune cell is a T cell, optionally a CD4+ T cell, a CD8+ T cell, or both.

97. The modified immune cell of any one of claims 88-96, wherein the modified immune cell is a CD4+ T cell and further comprises a heterologous polynucleotide encoding at least an extracellular portion of a CD8 co-receptor.

98. A composition comprising a modified immune cell of any one of claims 88-97 and a pharmaceutically acceptable carrier, diluent, or excipient.

99. A unit dose comprising an effective amount of (i) the modified immune cell of any one of claims 88-97 or (ii) a composition of claim 98.

100. The unit dose according to claim 99, comprising modified CD4+ CD25-T cells and modified CD8+ CD62L+ T cells in about a 1:1 ratio.

101. The unit dose of claim 99 or 100, wherein the unit dose comprises from about 10⁸ modified immune cells to about 10⁹ modified immune cells.

102. An adoptive immunotherapy method for treating a subject having a Merkel cell carcinoma, comprising administering to the subject an effective amount of a modified immune cell of any one of claims 88-97, a composition of claim 98, or a unit dose of any one of claims 99-101.

103. The method of claim 101, wherein the subject receives 1 or 2 unit doses of the modified immune cells.

104. The method of claim 102 or 103, wherein the subject is receiving an anti-PD-L1 antibody, optionally avelumab.

105. The method of any one of claims 102-104, wherein the subject has received, or is receiving, radiation therapy, optionally single-fraction radiation therapy.
